(19) United States
(12) Patent Application Publication
(10) Pub. No.: US 2005/0101542 A1
(43) Pub. Date: May 12, 2005

(54) Combination therapy for controlling appetites

(75) Inventors: Daniele Piomelli, Irvine, CA (US); Fernando Rodriguez de Fonseca, Madrid (ES); Jin Fu, Irvine, CA (US); Silvana Gaetani, Irvine, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California

(21) Appl. No.: 10/642,462

(22) Filed: Aug. 15, 2003

Related U.S. Application Data
(60) Provisional application No. 60/405,047, filed on Aug. 20, 2002.

(51) Int. Cl. A61K 31/70

(52) U.S. Cl. 514/23

(57) ABSTRACT

The invention provides methods and pharmaceutical compositions for administering a PPARα agonist (e.g., OEA-like agonist, OEA-like compound), an OEA-like appetite reducing compound, or a FAAH inhibitor and a CB1 cannabinoid receptor antagonist to a subject in order to reduce the consumption or ingestion of food, ethanol or other appetizing substances as well as in treating appetency disorders related to the excess consumption of food, ethanol, and other appetizing substances. The combination therapy can also be useful for reducing body fat or body weight and modulating lipid metabolism.

### CROSS-REFERENCE TO RELATED APPLICATIONS

This application claims benefit of U.S. Provisional Application No. 60/405,047, filed Aug. 20, 2002. This application contains subject matter related to U.S. Patent application Ser. No. 10/112,509, filed Mar. 27, 2002, which is a nonprovisional of U.S. Patent Application No. 60/336,289, filed Oct. 31, 2001 and of U.S. Patent Application No. 60/279,542, filed Mar. 27, 2001. This application also contains subject matter related to U.S. Provisional Patent Application No. 60/417,008, filed Oct. 7, 2002 and U.S. Provisional Patent Application No. 60/485,062, filed Jul. 2, 2003. Each of the above applications is assigned to the same assignee as the present application and the contents of each are incorporated herein by reference.

### STATEMENT AS TO RIGHTS TO INVENTIONS MADE UNDER FEDERALLY SPONSORED RESEARCH AND DEVELOPMENT

This invention was made with government support under Grant No. DA 12413, DA12447 and DA12653 awarded by the National Institutes of Health. The Government has certain rights in this invention.

### FIELD OF THE INVENTION

This invention relates to the pharmaceutical use of cannabinoid receptor antagonists in combination with PPAR-alpha agonists, including oleoylethanolamide and oleoylethanolamide-like fatty acid alkanolamide compounds, their homologues and their analogs to reduce excess or unwanted appetites or consumption of appetizing substances, such as foods, alcohol, and psychoactive substances of abuse.

### BACKGROUND OF THE INVENTION

Obesity is a worldwide health challenge occurring at alarming levels in the United States and other developed nations. About 97 million adults in the United States are overweight. Of these, 40 million are obese. Obesity and overweight greatly increase the risk of many diseases. Hypertension; type 2 diabetes; dyslipidemia; coronary heart disease; stroke; gallbladder disease; osteoarthritis; sleep apnea and other respiratory problems; and endometrial, breast, prostate, and colon cancers have been associated with higher body weights. Persons with higher body weights also suffer from a higher all-cause death rate. According to the National Institutes of Health, about 280,000 adult deaths in the United States each year may be attributed in part to obesity.

Weight loss is desirable in the case of obesity and overweight individuals. Weight loss can help to prevent many of these harmful consequences, particularly with respect to diabetes and cardiovascular disease (CVD). Weight loss may also reduce blood pressure in both overweight hypertensive and non-hypertensive individuals; serum triglycerides levels and increases the beneficial high-density lipoprotein (HDL)-form of cholesterol. Weight loss also generally reduces somewhat the total serum cholesterol and low-density lipoprotein (LDL)-cholesterol levels. Weight loss may also reduce blood glucose levels in overweight and obese persons.

While weight loss is desirable, it is hard to achieve. Many treatments for the management of overweight and obesity and the maintenance of weight loss exist. However, recidivism is rampant. Approximately 40 percent of women and 24 percent of men are trying to actively lose weight at any given time. These treatments include, but are not limited to, low-calorie diets and low-fat diets; increased physical exercise; behavioral therapies directed toward reducing food intake; pharmacotherapy; surgery; and combinations of the above.

The pharmacopeia of weight loss is relatively bare. A preferred way to reduce body weight is to reduce the appetite for foods and caloric beverages. Drugs such as sibutramine, dexfenfluramine, orlistat, phenylpropanolamine, phenteramine, or fenfluramine can facilitate weight loss in obese adults when used for prolonged periods. In general, however, the safety of long-term administration of pharmaco-therapeutic weight loss agents is unknown. For instance, recently due to concerns about valvular heart disease observed in patients, fenfluramine and dexfenfluramine have been withdrawn from the market. In the face of the slim pharmacopeia and the high prevalence of obesity and overweight, there is a need for new pharmaceutical methods and compositions to promote and maintain weight loss.

Historical descriptions of the stimulatory effects of Cannabis sativa on feeding are now explained by the ability of its psychoactive constituent Δ9-tetrahydrocannabinol (THC) to interact with CB1 cannabinoid receptors (Kunos and Batkai, Neurochem. Res., 26:1015-21 (2001); Williams, et al., Physiol. Behav., 65:343-6 (1998)). Both THC and the endogenous cannabinoid anandamide (Devane, et al., Science, 258:1946-1949 (1992)) promote overeating in partially satiated rats (Williams and Kirkham, Pshychopharmacology, 143:315-7 (1999)). Moreover, THC increases fat intake in laboratory animals and stimulates appetite in humans (Koch, Pharmacol. Biochem. Behav., 68:539-43 (2001); Sacks, et al., J. Am. Coll. Nutr., 9:630-632 (1990); Williams, et al., Physiol. Behav., 65:343:346 (1998)). The selective CB1 receptor antagonist SR141716A (Rinaldi-Carmona, et al., Life Sci., 56:1941-1947 (1995)) counteracts these effects and, when administered alone, decreases standard chow intake and caloric consumption—i.e., sucrose or ethanol intake—presumably by antagonizing the actions of endogenously released endocannabinoids such as anandamide and 2-arachidonoylglycerol (Arnone, et al., Psychopharmacology, 132:104-6, 1997; Colombo, et al., Alcohol, 33:126-30, 1998; Rowland, et al, Psychopharmacology, 159:111-6, 2001; Simiand, et al., Behav. Pharmacol, 9:179-81, 1998; Kirkham and Williams, Psychopharmacology, 153:267-70, 2001). These results suggest that endocannabinoid substances may play a role in the promotion of food intake, possibly by delaying satiety.

Anandamide, the naturally occurring amide of arachidonic acid with ethanolamine, meets all key criteria of an endogenous cannabinoid substance (Devane, et al., Science, 258: 1946-1949 (1992)): it is released upon demand by stimulated neurons (Di Marzo, et al., Nature, 372:686-691 (1994); Giuffrida, et al., Nat. Neurosci., 2:358-363 (1999)); it activates cannabinoid receptors with high affinity (Devane, et al., Science, 258: 1946-1949 (1992)) and it is rapidly eliminated through a two-step process consisting of carrier-mediated transport followed by intracellular hydrolysis (Di Marzo, et al., Nature, 372:686-691 (1994) (Beltramo, M. et al., FEBS Lett., 403:263-267 (1997)). Anandamide hydrolysis is catalyzed by the enzyme fatty acid amide hydrolase (FAAH), a membrane-bound serine hydrolase (Cravatt, B. F., et al., Nature, 384:83-87 (1996); Patricelli, M. P. et al., Biochemistry, 38:9804-9812 (1999)) (WO 98/20119) that also cleaves other bioactive fatty ethanolamides, such as oleoylethanolamide (oleamide; OEA; Z-2-hydroxyethyl octadec-9-enamide)) (Rodriguez de Fonseca, et al. Nature, 414:209-212 (2001)) and palmitoylethanolamide (Calignano, A., et al., Nature, 394:277-281 (1998)). Mutant mice lacking the gene encoding for FAAH cannot metabolize anandamide (Cravatt, B. F. et al., Proc. Natl. Acad. Sci. U.S.A., 98:9371-9376 (2001)) and, though fertile and generally normal, show signs of enhanced anandamide activity at cannabinoid receptors, such as reduced pain sensation (Cravatt, B. F. et al., Proc. Natl. Acad. Sci. U.S.A., 98:9371-9376 (2001)). This suggests the possibility that drugs targeting FAAH may heighten the tonic actions of anandamide and OEA, while possibly avoiding the multiple, often unwanted effects produced by Δ⁹-THC and other direct-acting cannabinoid agonists ((Hall, 1998 #13); Chaperon, 1999 #12]. In the presence of the CB-1 receptor the effects of FAAH inhibition mediated by endogenous anandamide are blocked while the synergistic effects of endogenous OEA are realized.

It is generally thought that the hyperphagic actions of cannabinoids are mediated by CB1 receptors located in brain circuits involved in the regulation of motivated behaviors (Herkenham, et al., J. Neurosci., 11:563-83 (1991)). Thus, infusions of anandamide in the ventromedial hypothalamus were shown to promote hyperphagia (Jamshidi and Taylor, Br. J. Pharmacol., 134:1151-4 (2001)), while the anorectic effects of leptin were found to be associated with a decrease in hypothalamic anandamide levels (Di Marzo, et al., Nature, 410:822-5 (2001)). Nevertheless, evidence suggests that cannabinoids also may promote feeding by acting at peripheral sites. Indeed, CB1 receptors are found on nerve terminals innervating the gastrointestinal tract (Croci, et al., Br. J. Pharmacol., 125:1393-5 (1998); Hohmann and Herkenham, Neuroscience, 90:923-931 (1999)), which are known to be involved in mediating satiety signals originated in the gut (Reidelberger, Am. J. Physiol., 263:R1354-R1358 (1992)). Others have also more reported that some cannabinoid antagonists can be useful in reducing appetites. (See, U.S. Pat. No. 6,344,474 to Maruani, et al., Feb. 5, 2002).

Peroxisome proliferator activated receptors (PPAR) are a family of transcription factors and have been postulated to play a role in lipid homeostasis. Three PPAR subtypes have been identified: α, β (also described as δ), and γ. All three subtypes have domain structure common with other members of the nuclear receptor family. DNA binding domains are highly conserved among PPAR subtypes, but ligand binding domains are less well conserved. (Willson, et al., J. Med. Chem., 43:527 (2000)).

PPARs bind to RXR transcription factors to form heterodimers that bind to DNA sequences containing AGGTCAnAGGTCA. It has been shown that ligand binding to PPAR can induce gene expression.

PPARγ is the best characterized of the three subtypes. Activation of PPARγ promotes adipocyte differentiation by repressing expression of the ob and TNFα genes. Activation of PPARγ also results in in vivo insulin sensitization. PPARγ has been implicated in several diseases including diabetes, hypertension, dyslipidemia, inflammation, and cancer.

PPARα is expressed at high levels in the liver, heart, renal cortex, brown fat, and intestine. PPARα regulates genes involved in almost all aspects of lipid metabolism and has been postulated to play a role in dyslipidemia, atherosclerosis, obesity, and diabetes.

PPARβ(δ) is the most widely expressed subtype and the least understood. PPARβ(δ) regulates acyl-coA synthetase 2 expression and is postulated to play a role in dyslipidemia, fertility, bone formation, and colorectal cancer. PPARβ(δ) expression in cells reduces their proliferation rate, but PPARβ expression in cells in conjunction with exposure to fatty acids increases proliferation rate.

All three subtypes are postulated to play a role in lipid homeostasis, but comparative studies have demonstrated significant differences among the subtypes. For example, mRNA expression of PPARα and PPARAγ is increased in ob/ob and db/db mice, but mRNA expression of PPARβ(δ) in ob/ob and db/db mice is the same as in control mice. It has also been shown that some ligands that bind to PPARγ and PPARα do not bind or activate PPARβ(δ).

As stated above, the PPAR family has been described as playing a role in obesity. Natural and synthetic subtype specific ligands have been identified for PPARα, PPARAγ, and PPARβ(δ). PPARα-selective compounds have an enhanced ability to reduce body fat and modulate fatty acid oxidation compared to PPARβ or PPARγ selective compounds. PPARα is activated by a number of medium and long-chain fatty acids. PPARα is also activated by compounds known as fibric acid derivatives. These fibric acid derivatives, such as clofibrate, fenofibrate, bezafibrate, ciprofibrate, beclofibrate and etofibrate, as well as gemfibrozil reduce plasma triglycerides along with LDL cholesterol, and they are primarily used for the treatment of hypertriglyceridemia.

Fatty acid ethanolamides (FAE) are unusual components of animal and plant lipids, and their concentrations in non-stimulated cells are generally low (Bachur, et al., J. Biol. Chem., 240:1019-1024 (1965); Schmid, et al., Chem. Phys. Lipids, 80:133-142 (1996); Chapman, K. D., Chem. Phys. Lipids, 108:221-229 (2000)). FAE biosynthesis can be rapidly enhanced, however, in response to a wide variety of physiological and pathological stimuli, including exposure to fungal pathogens in tobacco cells (Chapman, et al., Plant Physiol., 116:1163-1168 (1998)), activation of neurotransmitter receptors in rat brain neurons (Di Marzo, et al., Nature, 372:686-691 (1994); Giuffrida, et al., Nat. Neurosci., 2:358-363 (1999)) and exposure to metabolic stressors in mouse epidermal cells (Berdyshev, et al., Biochem. J, 346:369-374 (2000)). The mechanism underlying stimulus-dependent FAE generation in mammalian tissues is thought to involve two concerted biochemical reactions: cleavage of the membrane phospholipid, N-acyl phosphatidylethanolamine (NAPE), catalyzed by an unknown phospholipase D; and NAPE synthesis, catalyzed by a calcium ion- and cyclic AMP-regulated N-acyltransferase (NAT) activity (Di Marzo, et al., Nature, 372:686-691 (1994); Cadas, et al., J. NeuroSci., 6:3934-3942 (1996); Cadas, et al., H. J. Neurosci., 17:1226-1242 (1997)).

The fact that both plant and animal cells release FAEs in a stimulus-dependent manner suggests that these compounds may play important roles in cell-to-cell communication. Further support for this idea comes from the discovery that the polyunsaturated FAE, anandamide (arachidonylethanolamide), is an endogenous ligand for cannabinoid receptors (Devane, et al., Science, 258:1946-1949 (1992))—G protein-coupled receptors expressed in neurons and immune cells, which recognize the marijuana constituent Δ9-tetrahydrocannabinol (Δ9-THC) (for review, see reference (Pertwee, R. G., Exp. Opin. Invest. Drugs, 9:1553-1571 (2000)).

Two observations make it unlikely that other FAEs also participate in cannabinoid neurotransmission. The FAE family is comprised for the most part of saturated and monounsaturated species, such as palmitoylethanolamide and oleoylethanolamide, which do not significantly interact with cannabinoid receptors (Devane, et al., Science, 258:1946-1949 (1992); Griffin, et al., J. Pharmacol. Exp. Ther., 292:886-894. (2000)). Second, when the pharmacological properties of the FAEs have been investigated in some detail, as is the case with palmitoylethanolamide, such properties have been found to differ from those of Δ9-THC and to be independent of activation of known cannabinoid receptor subtypes (Calignano, et al., Nature, 394:277-281 (1998)). Thus, the biological significance of the FAEs remains elusive.

Oleoylethanolamide (OEA) (Z-2-hydroxyethyl octadec-9-enamide) is a natural analogue of the endogenous cannabinoid anandamide. Like anandamide, OEA is produced in cells in a stimulus-dependent manner and is rapidly eliminated by enzymatic hydrolysis, suggesting a role in cellular signaling. However, unlike anandamide, OEA does not activate cannabinoid receptors and its biological functions have only been recently discovered (Rodríguez de Fonseca, et al., Nature, 414: 209 212 (2001)).

Oleoylethanolamide is reported herein to be a potent and highly selective agonist of PPARα. With the discovery that OEA selectively modulates PPARα, the potential for using high throughput assays to identify other similar pharmacologically useful compounds which modulate PPARα is feasible. Such compounds will be useful in the treatment of PPARα-mediated diseases and conditions as well as any for which OEA was previously considered to be useful.

There is a need for additional methods and agents to treat obesity and overweight as well as to maintain weight loss. The present invention meets this need by providing novel methods and pharmaceutical compositions related to our instant discovery that PPARα modulators, including oleoylethanolamide (OEA) and other fatty acid alkanolamide compounds (e.g., palmitoylethanolamide, elaidoylethanolamide)) act synergistically with cannabinoid CB1 receptor antagonists to reduce appetite, food intake, body weight, and body fat and alter fat metabolism.

### SUMMARY OF THE INVENTION

The present invention relates to the surprising discovery that cannabinoid CB1 receptor antagonists and PPARα agonists (e.g., OEA), act synergistically to reduce appetite(s) and promote weight loss when administered to the same subject. The invention provides pharmaceutical compositions, compounds, and methods for reducing appetite(s), reducing body fat and for treating or preventing obesity or overweight in a mammal and for preventing or treating the diseases associated with these health conditions. In one aspect of the instant invention, methods are provided for reducing appetite, body fat or body weight, or for treating or preventing obesity or overweight, or for reducing food intake or consumption, or treating an appetency disorder in a mammal by administering to the mammal a combination therapy providing both 1) a cannabinoid CB1 receptor antagonist and 2) a PPARα receptor agonist (e.g., an OEA-like PPARα agonist, an OEA-like compound) Or an OEA-like appetite reducing compound or a FAAH inhibitor.

In one embodiment, the cannabinoid receptor antagonist and the PPARα agonist (e.g., OEA, fatty acid alkanolamide compound, or homologue or analog of OEA or the fatty acid alkanolamide having PPARα agonist activity) are administered to a subject in amounts sufficient to reduce body fat, body weight, or prevent body fat or body weight gain or to reduce appetite(s).

In another embodiment, the PPARα agonist is clofibrate or a derivative of clofibrate. Such derivatives would include, but not be limited to, clofibrate; fenofibrate, bezafibrate, gemfibrozil, and ciprofibrate. In a further embodiment, the cannabinoid receptor antagonist to be co-administered or co-formulated with the PPARα agonist is rimonabant.

In another aspect of the invention, pharmaceutical compositions are provided which comprise a first compound which is an antagonist of the CB1 cannabinoid receptor and a second compound which is oleoylethanolamide (OEA) Or a fatty acid alkanolamide compound, or a homologue or analog of oleoylethanolamide or the fatty acid alkanolamide compound which reduces appetite or acts as an agonist at the PPARα receptor. In other aspects, the invention is drawn to such pharmaceutical compositions and their methods of use to reduce or control appetite or to treat appetite disorders.

In some aspects, the invention provides method of treating an appetency disorder comprising administration of a first compound which is a CB1 cannabinoid receptor antagonist and a second compound which is an agonist of the PPARα receptor (e.g., a OEA-like compound; an OEA-like PPARα agonist); or an OEA-appetite reducing compound, a fatty acid alkanolamide compound, homologue or OEA analog which is not a significant antagonist of the cannabinoid CB1 receptor (i.e., can be administered in therapeutic amounts which do not by themselves significantly activate or inhibit the CB1 receptor)). In another aspect of the invention, pharmaceutical compositions are provided which comprise a first compound which is an antagonist of the CB1 cannabinoid receptor and a second compound which is oleoylethanolamide (OEA) Or a fatty acid alkanolamide compound, or a homologue or analog of oleoylethanolamide or the fatty acid alkanolamide compound, which is not a significant CB1 cannabinoid receptor antagonist and which reduces appetite or which has an effect to reduce appetite which is not substantially mediated by binding of the second compound to the CB1 cannabinoid receptor. In other aspects, the invention is drawn to such pharmaceutical compositions and their methods of use to reduce or control appetite and to treat appetite disorders.

In one embodiment, the cannabinoid antagonist is administered with the PPARα agonist or OEA-like appetite reducing compound in amounts which act synergistically. In one embodiment, these amounts are subthreshold amounts for both the individual antagonist and the OEA-like PPARα agonist, OEA-like compound, or OEA-like appetite reducing compound. In one embodiment, the cannabinoid antagonist and the OEA-like PPARα agonist, OEA-like compound or OEA-like appetite reducing compound are formulated in a single pharmaceutical composition in unit dosage format in which the unit dose contains the cannabinoid receptor antagonist and the OEA-like PPARα agonist, OEA-like compound, or OEA-like appetite reducing compounds each in an amount which can act synergistically with the other compound upon administration. In a still further embodiment, these unit dose amounts are individually subthreshold amounts or near subthreshold amounts for both the individual CB1 cannabinoid receptor antagonist and the individual OEA-like PPARα agonist, OEA-like compound, or OEA-like appetite reducing compound. In a still further embodiment, the fatty acid alkanolamide compound, homologue or analog is OEA.

In one embodiment, the CB1 cannabinoid antagonist is selective for the CB1 cannabinoid receptor as opposed to the CB2 cannabinoid receptor. In another embodiment, the cannabinoid receptor antagonist is a aryl-benzo[b]thiophene or aryl-benzo[b]furan derivative which is an antagonist of the cannabinoid CB1 receptor as taught in U.S. Pat. No. 5,596,106.

In one embodiment, the CB1 receptor cannabinoid antagonist is SR141716 or a physiologically compatible salt thereof. In one embodiment, the cannabinoid antagonist is SR141716A or rimonabant.

In one embodiment, the CB1 cannabinoid antagonist is a 4,5,dihydro-1H-pyrazole derivative having CB1-antagonist activity as taught in U.S. Pat. No. 5,747,524 and U.S. Patent Application No. 2001/0053788A1 published on Dec. 20, 2001.

In another embodiment, the cannabinoid receptor antagonist has the formula as taught in Formula I of U.S. Pat. No. 6,017,919.

In another embodiment, the OEA-like PPARα agonist, OEA-like compound, or OEA-like appetite reducing compound is a fatty acid alkanolamide. In a further embodiment, the alkanolamide moiety is ethanolamide.

In another embodiment, the PPARα agonist, OEA-like PPARα agonist, OEA-like compound or OEA-like appetite reducing compound is not an antagonist of the CB1 cannabinoid receptor.

In another embodiment, the OEA-like agonist, OEA-like compound or OEA-like appetite reducing compound does not significantly occupy the CB1 cannabinoid receptor activity when administered in amounts according to the present invention. In a further embodiment, the OEA-like appetite reducing compound has an IC₅₀ for binding to the CB1 cannabinoid receptor which is greater than 10 μM. In another embodiment, the IC₅₀ for binding to the CB1 cannabinoid receptor is greater than 100 μM.

In other embodiments, the OEA-like PPARα agonist or OEA-like compound or OEA-like appetite reducing compound (e.g., a fatty acid alkanolamide or ethanolamide compound, homologue or analog of OEA or the fatty acid alkanolamide), is not significantly a cannabinoid CB1 receptor antagonist. In another embodiment, the fatty acid alkanolamide or ethanolamide compound, homologue or analog of OEA is administered in an amount which would not appreciably antagonize the CB1 cannabinoid receptor if administered alone.

In other embodiments, the OEA-like compound, OEA-like agonist, or OEA-like appetite reducing compound is a fatty acid alkanolamide or ethanolamide compound, homologue, or analog in which the fatty acid moiety may be saturated or unsaturated, and if unsaturated may be monounsaturated or polyunsaturated.

In some embodiments, the PPARα agonist is a fatty acid alkanolamide compound, homologue, or analog having a fatty acid selected from the group consisting of oleic acid, palmitic acid, elaidic acid, palmitoleic acid, linoleic acid, alpha-linolenic acid, and gamma-linolenic acid. In certain embodiments, the fatty acid moieties have from twelve to 20 carbon atoms with, in some embodiments, 0, 1, 2, 3, or 4 double bonds.

Other embodiments are provided by varying the hydroxyalkylamide moiety of the OEA-like fatty acid amide compound, homologue or analog. These embodiments include, but are not limited to, the introduction of a substituted or unsubstituted lower (C₁-C₃) alkyl group on the hydroxyl group of an alkanolamide or ethanolamide moiety so as to form the corresponding lower alkyl ether. In another embodiment, the hydroxy group of the alkanolamide or ethanolamide moiety is bound to a carboxylate group of a C₂ to C₆ substituted or unsubstituted cyclic or acyclic carboxylic acid to form the corresponding ester of the fatty acid ethanolamide. Such embodiments include, but are not limited to, fatty acid alkanolamide and fatty acid ethanolamides in ester linkage to organic carboxylic acids such as acetic acid, propionic acid, butyric acid and pivalic acid. In one embodiment, the fatty acid alkanolamide is an oleoylalkanolamide. In one embodiment, the fatty acid alkanolamide is oleoylethanolamide. In another embodiment, the fatty acid alkanolamide is palmitoylethanolamide.

In still another embodiment, the OEA-like fatty acid ethanolamide compound, homologue, or analog further comprises a substituted or unsubstituted lower alkyl (C₁-C₃) group covalently bound to the nitrogen atom of the fatty acid ethanolamide.

In another aspect, the invention provides a pharmaceutical composition comprising a pharmaceutically acceptable excipient or carrier and a first compound which is a CB1 receptor antagonist and a second compound which is a PPARα agonist or appetite reducing compound, or a pharmaceutically acceptable salt thereof, having the formula: 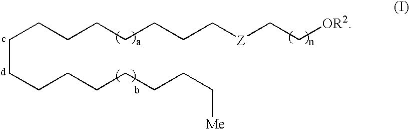
In this formula, n is from 0 to 5 and the sum of a and b can be from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of substituted or unsubstituted alkyl, hydrogen, substituted or unsubstituted C₁-C₆ alkyl, substituted or unsubstituted lower (C₂-C₆) acyl, (C₁-C₆) homoalkyl, and aryl. Up to four hydrogen atoms of either or both the fatty acid portion and alkanolamine portion of the compound may also be substituted by a methyl group or by a double bond replacing H on adjacent carbons. In addition, the molecular bond between carbons c and d may be unsaturated or saturated. In some embodiments, the fatty acid ethanolamide of the above formula is a naturally occurring compound.

In another embodiment, the compound of formula I is not a CB1 receptor antagonist or acts at therapeutic dosages to reduce appetite principally other than through binding of the compound to the CB1 cannabinoid receptor.

In another embodiment, the pharmaceutical composition is in unit dosage format and comprises both a CB1 cannabinoid receptor antagonist and a compound of the instant formula I in a pharmaceutically acceptable carrier. In a further embodiment the amount of the CB1 antagonist or the compound of formula I in the unit dosage would, by itself, not be effective for controlling appetite.

In another embodiment the pharmaceutical composition comprises SR141716 and a compound of formula I, or a pharmacologically acceptable salt thereof. In a further embodiment, the compound of formula I is oleoylethanolamide.

In another embodiment, the cannabinoid receptor antagonist has a peripheral site of action via a peripheral CB1 receptor upon administration to a mammal. In another embodiment, the CB1 cannabinoid receptor antagonist is selective for a peripheral CB1 receptor upon systemic administration. In another embodiment, the CB1 cannabinoid receptor is administered in amounts below those which significantly antagonize the central CB1 receptors. In another embodiment, the CB1 antagonist is selected according to a relative inability to cross the blood brain barrier. In another embodiment, the CB1 cannabinoid receptor antagonist bears a net charge at physiological pH. In another embodiment, the central concentration (e.g., in the cerebrospinal fluid) Of the administered CB1 cannabinoid receptor antagonist is 4-fold less than that of the peripheral concentration (e.g., in the plasma or serum).

In other aspects of the invention, the methods and compositions employ below threshold or near-threshold amounts of the OEA-like agonist, OEA-like compound or OEA-like appetite reducing compound in which such compound can cause reduced appetite, reduced food consumption or weight loss when administered to test animals (e.g., rats, mice, rabbits, hamsters, guinea pigs) Or humans in larger than threshold amounts.

In still other aspects, the invention is drawn to methods of using cannabinoid CB1 receptor antagonists and arylthiazolidinedione compounds and heteroaryl and aryl oxyacetic acid type compounds in combination with a CB1 cannabinoid receptor antagonist to reduce appetite.

In another aspect, the invention provides peripherally acting fatty acid alkanolamides and the homologues and analogs thereof to reduce appetite. These agents are preferably administered in a combination therapy with a cannabinoid receptor antagonist to reduce appetite or an appetency disorder. In a further embodiment, the CB1 cannabinoid antagonist is a peripherally acting CB1 cannabinoid receptor antagonist. The selectivity for a peripheral site of action can be based upon a reduced rate or ability to cross the blood brain barrier or a selectivity for the CB1 cannabinoid receptor itself.

In another aspect, the invention provides a combination therapy and formulations of OEA-like compounds, OEA-like PPARα agonists, and OEA-like appetite reducing compounds with with CB1 receptor antagonists which can act synergistically to reduce appetite for food or to treat an appetency disorder.

In another aspect, the invention employs a fatty acid amide hydrolase inhibitor in an amount sufficient to increase the level of endogenous OEA such that the administered FAAH inhibitor acts synergistically with an administered amount of a CB1 cannabinoid receptor antagonist to reduce appetite for food or to treat an appetency disorder. In one aspect, the invention is drawn to a pharmaceutical composition comprising a FAAH inhibitor and a CB-1 cannabinoid receptor antagonist.

Still other aspects of the invention address methods of using and administering the subject cannabinoid receptor antagonists and PPARα agonists or OEA-like appetite reducing compounds in a combination therapy for reducing body weight or reducing body fat or reducing appetite for food or reducing food intake or consumption or causing hypophagia in mammals (e.g., humans, cats or dogs). The subject compositions may be administered by a variety of routes, including orally.

Still other aspects of the invention provide methods for reducing appetites or treating appetency disorders related to drug and alcohol abuse. In one embodiment, inventive methods and compositions are used to suppress the increased appetite associated with nicotine or tobacco withdrawal. In another embodiment, the inventive methods and compositions are used to treat addiction to psychoactive substances such as narcotics, CNS stimulants, CNS depressants, and anxyiolytics.

### BRIEF DESCRIPTION OF THE DRAWINGS

FIG. 1. Starvation increases circulating oleoylethanolamide levels in rats: (a) time course of the effects of food deprivation on plasma oleoylethanolamide (OEA) levels; (b) effect of water deprivation (18 h) On plasma oleoylethanolamide levels; (c) effect of food deprivation (18 h) On oleoylethanolamide levels in cerebrospinal fluid (CSF); (d) time course of the effects of food deprivation on plasma anandamide (arachidonylethanolamide, AEA) levels; (e) effect of water deprivation (18 h) On anandamide plasma levels; (f) effect of food deprivation (18 h) On anandamide levels in CSF. Results are expressed as mean±s.e.m.; asterisk, P<0.05; two asterisks, P<0.01, n=10 per group.

FIG. 2. Adipose tissue is a primary source of circulating oleoylethanolamide: starvation-induced changes in N-acyltransferase (NAT) and fatty acid amide hydrolase (FAAH) activities in various rat tissues. (a) fat; (b) brain; (c) liver; (d) stomach; (e) small intestine. Empty bars, free-feeding animals; filled bars, 18-h fasted animals. Activities are in pmol/mg protein/min. Asterisk, P<0.05, n=3.

FIG. 3. Adipose tissue is a primary source of circulating oleoylethanolamide: starvation-induced changes in NAPE and oleoylethanolamide (oleoylethanolamide, OEA) content in adipose and liver tissues. (a) structures of the oleoylethanolamide precursors alk-1-palmitoenyl-2-arachidonyl-sn-glycero-phosphoethanolamine-N-oleyl (left panel, NAPE 1) and alk-1-palmityl-2-arachidonyl-sn-glycero-phosphoethanolamine-N-oleyl (right panel, NAPE 2); (b) representative HPLC/MS tracings for selected ions characteristic of NAPE 1 (left panel, m/z=987, deprotonated molecule, [M−H]⁻) and NAPE 2 (right panel, m/z=1003, [M−H]⁻) in free-feeding (top) and 18-h fasting rats (bottom); (c) food deprivation (18 h) increases the content of NAPE species in fat and decreases it in liver. All identifiable NAPE species were quantified, including the oleoylethanolamide precursors NAPE1 and NAPE 2, and the PEA precursor NAPE 3; (d) food deprivation (18 h) increases oleoylethanolamide content in fat and liver. Empty bars, free-feeding animals; filled bars, 18-h fasted animals. Asterisk, P<0.05, Student's t test; n=3.

FIG. 4. Oleoylethanolamide (OEA/pranamide) selectively suppresses food intake: (a) dose-dependent effects of oleoylethanolamide (i.p., empty squares), elaidoylethanolamide (empty circles), PEA (triangles), oleic acid (filled squares) and anandamide (filled circles) On food intake in 24-h food-deprived rats. Vehicle alone (70% DMSO in saline, 1 ml per kg, i.p.) had no significant effect on acute food intake; (b) time course of the hypophagic effects of oleoylethanolamide (20 mg per kg, i.p.) (squares) Or vehicle (lozenges) On food intake. (c) effects of vehicle (V), lithium chloride (LiCl, 0.4 M, 7.5 ml per kg) Or oleoylethanolamide (20 mg per kg) in a conditioned taste aversion assay. Empty bars, water intake; filled bars, saccharin intake. Effects of vehicle (V) Or oleoylethanolamide (5 or 20 mg per kg) On: (d) water intake (expressed in ml per 4 h); (e) body temperature; (f) latency to jump in the hot plate analgesia test; (g) percent time spent in open arms in the elevated plus maze anxiety test; (h) Number of crossings in the open field activity test; (i) Number of operant responses for food. Asterisk, P<0.05, n=8-12 per group.

FIG. 5. Effects of subchronic oleoylethanolamide administration on food intake and body weight: (a) effects of oleoylethanolamide (OEA) (5 mg per kg, i.p. once a day) (empty bars) Or vehicle (5% Tween 80/5% polyethyleneglycol in sterile saline; filled bars) On cumulative food intake; (b) time course of the effects of oleoylethanolamide (triangles) Or vehicle (squares) On body weight change; (c) effects of oleoylethanolamide or vehicle on net body weight change; (d) effects of oleoylethanolamide (5 mg per kg) Or vehicle on cumulative water intake. Asterisk, P<0.05; two asterisks, P<0.01, n=10 per group.

FIG. 6. Role of peripheral sensory fibers in oleoylethanolamide-induced anorexia. Effects of vehicle (V), oleoylethanolamide (oleoylethanolamide/pranamide/OEA) (5 mg per kg, i.p.), CCK-8 (10 μg per kg) and CP-93129 (1 mg per kg), a centrally active 5-HT_{1B} receptor agonist, on food intake in a, control rats and c, capsaicin-treated rats. Water intake in (b) control rats and (d) capsaicin-treated rats. Asterisk, P<0.05; n=8-12 per group.

FIG. 7. Oleoylethanolamide increases c-fos mRNA expression in discrete brain regions associated with energy homeostasis and feeding behavior: (a) pseudocolor images of film autoradiographs show that oleoylethanolamide (right section) elicits a striking and selective increase in c-fos mRNA labeling in the paraventricular (PVN) and supraoptic (SO) hypothalamic nuclei, as assessed by in situ hybridization. A representative section from a vehicle-treated rat is shown at left. Labeling densities are indicated by color: blue<green<yellow<red. (b) quantification of c-fos cRNA labeling in forebrain regions [PVN, SO, arcuate (Arc), layer II piriform cortex (pir), ventrolateral thalamas (VI) and S1 forelimb cortex (S1FL)] of rats treated with vehicle, oleoylethanolamide and oleic acid; (c) film autoradiogram showing elevated ³⁵S c-fos mRNA expression in the nucleus of the solitary tract (NST) in an oleoylethanolamide-treated rat; Inset, c-fos cRNA labeling in the NST (shown in red) was identified by its localization relative to adjacent efferent nuclei (hypoglossal and dorsal motor nucleus of the vagus), which express choline acetyl transferase (ChAT) mRNA (shown in purple); (d) Oleoylethanolamide increases c-fos mRNA expression in NST but not in the hypoglossal nucleus (HgN). Two asterisks, P<0.0001, n=5 per group.

FIG. 8. The effects of OEA, Oleic acid (OA), AEA, PEA, and methyl-OEA on fatty acid oxidation in soleus muscle.

FIG. 9. Activation of human PPARα-GAL4 chimeric receptors by OEA. a, Concentration-dependent effects of OEA on PPARα (closed circles), PPARδ (open triangles), PPARγ (closed squares) and RXR (open lozenges). b, Effects of OEA (closed circles), oleic acid (open squares), stearylethanolamide (closed triangles), myristylethanolamide (closed squares), and anandamide (open circles) On PPARα activation. Results are the mean±s.e.m. of n=16.

FIG. 10. OEA reduces feeding in wild-type mice, but not in mice deficient for PPAR-α. Time course of the hypophagic effects of OEA (10 mg-kg⁻¹, i.p.) (closed squares) or vehicle (70% DMSO in saline, 1 ml-kg⁻¹, i.p)(open squares) On cumulative food intake normalized for body weight in a, wild-type mice, and b, PPAR-α-null mice. c, Effects of vehicle (V), d-fenfluramine (4 mg-kg⁻¹, i.p.) Or cholecystokinin-octapeptide (25 μg-kg⁻¹, i.p.) on cumulative food intake in wild-type (+/+) and PPAR-α-null (−/−) mice. Asterisk, P<0.05; n=8-12 per group.

FIG. 11. Subchronic OEA administration reduces food intake and body mass in wild-type, but not in PPAR-α null mice. Effects of OEA (5 mg mg-kg⁻¹, i.p.) (solid bars) Or vehicle (propylenglycol/Tween80/saline, May 5, 1990; 1 ml-kg⁻¹, i.p.) (open bars) On a, cumulative food intake normalized for body weight; b, cumulative body-weight gain; c, liver tissue triglycerides; d, white adipose tissue triglycerides; and e, serum cholesterol, in wild-type (+/+) and PPAR-α-null (−/−) mice. Asterisk, P<0.05; Two asterisks, P<0.001; n=10 per group.

FIG. 12. Synthetic PPAR-α agonists mimic the satiety-inducing actions of OEA. a, Effects of vehicle (open squares), Wy-14643 (closed triangles) (40 mg kg⁻¹, i.p.) and GW-7647 (open circles) (20 mg kg⁻¹, i.p.) On cumulative food intake normalized for body weight in C57BL/6J mice (vehicle, n=40; drugs, n=4-7). b, Effects of vehicle (V, open bars), Wy-14643 (W) (40 mg kg⁻¹, i.p.), GW-7647 (G₁) (20 mg kg⁻¹, i.p.) and OEA (O) (10 mg kg⁻¹, i.p.) on feeding latency, first meal size (MS) and first post-meal interval (PMI) in C57BL/6J mice (vehicle, n=40; drugs, n=4-7). c, Effects of vehicle (V, open bars), OEA (O) (10 mg kg⁻¹, i.p.) and d-fenfluramine (F) (3 mg kg⁻¹, s.c.) On food intake in control rats (sham, n=5-8) and vagotomized rats (vag, n=5-6). d-e, Time-course of the effects of vehicle (open symbols) Or Wy-14643 (closed symbols) (40 mg kg⁻¹, i.p.) On food intake in d, control rats (n=7-8) and e, vagotomized rats (n=5-6). f, Lack of effect of the PPAR-⁻β/δ agonist GW501516 (G₂) (5 mg kg⁻¹, i.p.) and PPAR-γ agonist ciglitazone (C) (15 mg kg⁻¹, i.p.) On cumulative food intake in C57BL/6J mice (vehicle, n=40; drugs, n=4-6 per group). g-h, Time-course of the effects of vehicle (open symbols) Or Wy-14643 (closed symbols) (40 mg kg⁻¹, i.p.) On cumulative food intake normalized for body weight in g, wild-type mice (n=8-11) and h, PPAR-α null mice (n=7-8). Asterisk, P<0.05; two asterisks, P<0.001; three asterisks, P<0.0001; one-way ANOVA followed by Dunnett's test or, when appropriate, t-test with Bonferroni's correction.

FIG. 13. OEA regulates gene expression in the jejunum and liver of wild-type but not PPAR-α null mice. a-g, Activation of gene expression by OEA in a-d, jejunum; e-g, liver. a-e, Effects of vehicle (V, open bar), Wy-14643 (W) (30 mg kg⁻¹, i.p.) Or OEA (O) (10 mg kg⁻¹, i.p.) On mRNA levels of a, PPAR-α; b, FAT/CD36; c, FATP1; and d, PPAR-δ, PPAR-γ and I-FABP in the jejunum of wild-type (+/+) and PPAR-α null (−/−) mice (n=5 per group). e-g, Effects of vehicle (V, open bars), Wy-14643 (W) (30 mg kg⁻¹, i.p.) Or OEA (O) (10 mg kg⁻¹, i.p.) On mRNA levels of e, PPAR-α; f, FAT/CD36; and g, liver-FABP in wild-type (+/+) and PPAR-α null (−/−) mice (n=5 per group). h, Transrepression of iNOS expression by OEA (O) (10 mg kg⁻¹, i.p.) and Wy-14643 (W) (30 mg kg⁻¹, i.p.) in the jejunum of C57BL/6J mice (n=5). mRNA levels are expressed in arbitrary units. Asterisk, P<0.05; two asterisks, P<0.001; one-way ANOVA followed by Dunnett's test.

FIG. 14. OEA initiates expression of PPAR-α-regulated genes in the duodenum of wild-type but not PPAR-α-null mice. a, Time course of the effects of vehicle (open bars) or OEA (solid bars) (10 mg kg⁻¹, i.p.) On PPAR-α mRNA levels in the duodenum of C57BL/6J mice (n=5 per group). b-e, Effects of vehicle (V, open bar), Wy-14643 (W) (30 mg kg⁻¹, i.p.) Or OEA (O) (10 mg kg⁻¹, i.p.) On mRNA levels of b, PPAR-α; c, FAT/CD36; d, FATP1; and e, PPAR-δ, PPAR-γ and I-FABP in wild-type (+/+) and PPAR-α-null (−/−) mice (n=5 per group). mRNA levels were measured as described under Methods and are expressed in arbitrary units. Asterisk, P<0.05; two asterisks, P<0.001.

FIG. 15. OEA and synthetic PPAR-α agonists fail to induce expression of PPAR-α-regulated genes in the ileum of wild-type and PPAR-α-null mice. Effects of vehicle (V, open bars), Wy-14643 (W) (30 mg kg⁻¹, i.p.) Or OEA (O) (10 mg kg⁻¹, i.p.) On mRNA levels of a, PPAR-α; b, FAT/CD36; c, FATP1; and d, PPAR-δ, PPAR-γ and I-FABP in wild-type (+/+) and PPAR-α-null (−/−) mice (n=5 per group). mRNA levels were measured as described under Methods and are expressed in arbitrary units. Asterisk, P<0.05; two asterisks, P<0.001.

FIG. 16. Concerted regulation of intestinal OEA synthesis and PPAR-α expression. a, Food intake; b, OEA content; c, PPAR-α mRNA levels; and d, iNOS mRNA levels at night-time (1:30 AM; closed bars) and daytime (4:30 PM; open bars) in free-feeding C57Bl/6J mice maintained on a 12:12 dark/light cycle (n=3). Asterisk, P<0.05; two asterisks, P<0.001; Student's t-test.

FIG. 17. Effect of subhronic OEA administration (5 mg/kg, once daily for 2 weeks, i.p.) On food intake and body weight gain over the two week period. Black circles, OEA. Open squares, vehicle.

FIG. 18. Effects of starvation and feeding on anandamide levels in the brain and small intestine. Starvation promoted the accumulation of anandamide in the small intestine. Data are the means±SEM of at least 5 determinations per group. (*) P<0.01, fed versus starved group, Newman-Keuls.

FIG. 19. Peripheral effects of cannabinoids on food intake. A. Anandamide (AEA) elicited hyperphagia in partially satiated animals when injected after a 60 min meal. B. Anandamide has no effect after i.c.v. administration. C. Acute i.p. injection of WIN 55,212-2 (WIN) promoted hyperphagia in partially satiated animals. D. WIN 55,212-2 has no effect after i.c.v. injection E. Acute i.p. injection of SR141716A reduced food intake in food-deprived rats during the 240-min testing period. F. The i.c.v. administration of SR141716A did not affect food intake in food-deprived animals. Data are the means±SEM of at least 10 determinations per group. (*) P<0.01, versus vehicle-treated group (white bars), Newman-Keuls.

FIG. 20. A. Capsaicin treatment abolished the anorexic effect of CCK-8, which acts peripherally, but not those of the 5HT-1B agonist CP 93129, which acts centrally. B. WIN 55,212-2 did not produce hyperphagia. C. Capsaicin treatment abolishes the reduction of food intake elicited by SR141716A in food deprived rats. Data are the means±SEM of at least 10 determinations per group. (*) P<0.01, versus vehicle-treated group, Newman-Keuls

FIG. 21. Synergistic effects of SR141716A and OEA on feeding suppression. Effects of subthreshold doses of SR141716A (0.3 mg/kg i.p,) and OEA (0.5 and 1 mg/kg i.p.) on food intake in 24 hr food.-deprived rats, A. 2 h after injection of OEA and B. 24 h. after injection of OEA. Either vehicle (open bars) Or SR141716A (black bars) were injected 30 min prior to OEA. Data are the means±SEM of at least 10 determinations per group. (*) P<0.01, versus vehicle-treated group, Newman-Keuls.

### DETAILED DESCRIPTION OF THE INVENTION

OEA and other OEA-like fatty acid alkanolamide compounds and OEA analogs and homologs reduce appetite, food intake, body weight, and body fat and modulate fatty acid oxidation. These effects are not thought to be significantly due to a direct interaction of such compounds with the CB1 cannabinoid receptor.

As disclosed in co-pending U.S. Provisional Patent Application No. 60/485,062, filed on Jul. 2, 2003, assigned to the same assignee as the present application, and incorporated by reference in its entirety to the extent not inconsistent with the present application, it has been advantageously discovered that:
(1) OEA selectively engages with high affinity the peroxisome proliferator-activating receptor alpha (PPARα), a ligand-operated transcription factor that regulates multiple aspects of lipid metabolism.
(2) Administration of OEA produces satiety and reduces body-weight gain in wild-type mice, but not in mice deficient in PPARα.
(3) Two structurally distinct, high-affinity PPARα agonists exert similar effects, which also are contingent on PPARα expression; and that, in contrast, potent and selective agonists for PPARγ and PPARδ are ineffective.
(4) In the small intestine and liver of wild-type, but not PPARα null mice, OEA initiates transcription of several PPARα regulated genes, including those encoding for the fatty acid transporters FATP1 and FAT/CD36.

The above findings indicate that OEA induces satiety by acting as a high-affinity ligand for PPARα and suggest a role for OEA signaling via PPARα in the regulation of lipid metabolism. The results further indicate the importance of PPARα in the mediation of diseases and conditions related to body fat burden, obesity, metabolic disorders, and appetite. The results further show that OEA-like compounds, including but not limited to, fatty acid alkanolamides and homologs thereof can be potent and selective PPARα modulators. Such modulators find use in the treatment of diseases and conditions mediated by PPARα (e.g., diseases responsive to administration of agonists of PPARα). The results further indicate the high affinity specific PPARα agonists or OEA-like modulators are particularly useful in the treatment of appetite disorders, obesity, and in reducing body fat and body weight.

CB1 receptor antagonists have also been reported to suppress appetitive behavior in test animals. For instance, the selective CB1 receptor antagonist SR141716A (Rinaldi-Carmona, et al., Life Sci., 56:1941-1947 (1995)) Counteracts the effects of CB1 receptor agonists and, when administered alone, decreases standard chow intake and caloric consumption. Others have also more reported that some cannabinoid antagonists can be useful in reducing appetites. (See, U.S. Pat. No. 6,344,474 to Maruani, et al., Feb. 5, 2002).

This invention relates to the surprising discovery that CB1 receptor blockade synergistically potentiates (e.g., provides the combined effects that are greater than the sum of the individual effects for each compound). The suppression of feeding evoked by OEA which was later determined to be an endogenous PPARα agonist.

### Definitions

Each publication, Patent application, Patent, and other reference cited herein is incorporated by reference in its entirety to the extent that it is not inconsistent with the present disclosure.

It is noted here that, as used in this specification, the singular forms “a,” “an,” and “the” include plural reference unless the context clearly dictates otherwise. The terms “include(s)” or “including” are non-limiting (e.g., “including” may be read for instance, as reciting, “including, but are not limited to”).

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

In the present description and in the claims, “appetency disorders” are understood as meaning disorders associated with a substance and especially abuse of a substance and/or dependency on a substance, disorders of food behaviors, especially those liable to cause excess weight, irrespective of its origin, for example: bulimia, appetency for sugars, non-insulin-dependent diabetes. Appetizing substances are therefore understood as meaning substances to be taken into the body and for which an appetite or craving for such consumption by any route of entry. Appetizing substances include, but are not limited to, foods, and their appetizing ingredients such as sugars, carbohydrates, or fats, as well as drinking alcohol or drugs of abuse or excess consumption. An “appetite’ may be directed toward such substances as foods, sugars, carbohydrates, fats, as well as ethanol or drugs of abuse or addiction or excess consumption (e.g., tobacco, CNS depressants, CNS stimulants).

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention (e.g., the OEA-like agonist, OEA-like compound or OEA-like appetite reducing compound, cannabinoid receptor antagonist, FAAH inhibitor) and a pharmaceutically acceptable carrier. The term “pharmaceutical composition” indicates a composition suitable for pharmaceutical use in a subject, including an animal or human. A pharmaceutical composition generally comprises an effective amount of an active agent and a pharmaceutically acceptable carrier.

The term “body fat reduction” means loss of a portion of body fat.

The formula for Body Mass Index (BMI) is [Weight in pounds÷Height in inches÷Height in inches]×703. BMI cutpoints for human adults are one fixed number, regardless of age or sex, using the following guidelines: Overweight human adults individuals have a BMI of 25.0 to 29.9. Obese human adults have a BMI of 30.0 or more. Underweight adults have a BMI less of than 18.5. A normal body weight range for an adult is defined as a BMI between 18.5 and 25. BMI cutpoints for children under 16 are defined according to percentiles: Overweight is defined as a BMI for age greater than >85th percentile and obesity is defined as a BMI-for-age>95th percentile. Underweight is a BMI-for-age<5th percentile. A normal body weight range for a child is defined as a BMI above the 5th percentile and below the 85 percentile.

The term “fatty acid oxidation” relates to the conversion of fatty acids (e.g., oleate) into ketone bodies.

The term “hepatocytes” refers to cells originally derived from liver tissue. Hepatocytes may be freshly isolated from liver tissue or established cell lines.

The term “modulate” means to induce any change including increasing or decreasing. (e.g., a modulator of fatty acid oxidation increases or decreases the rate of fatty oxidation, a modulator of a receptor includes both agonists and antagonists of the receptor).

The term “muscle cells” refers to cells derived from the predominant cells of muscle tissue. Muscle cells may be freshly isolated from muscle tissue or established cell lines.

The term “weight loss” refers to loss of a portion of total body weight.

Fatty acid amide hydrolase (FAAH) is the enzyme primarily responsible for the hydrolysis of anandamide in vivo. It also is responsible for the hydrolysis of OEA in vivo. Inhibitors of the enzyme are well known to one of ordinary skill in the art (Cravatt, B. F. et al., Nature, 384:83-87 (1996); Patricelli, M. P. et al., Biochemistry, 38:9804-9812 (1999); WO Patent Publication No. 98/20119; Rodríguez de Fonseca, et al. Nature, 414:209-212 (2001); Calignano, et al., Nature, 394:277-281 (1998)). Mutant mice lacking the gene encoding for FAAH cannot metabolize anandamide (Cravatt, B. F. et al., Proc. Natl. Acad. Sci. U.S.A., 98:9371-9376 (2001)) and, though fertile and generally normal, show signs of enhanced anandamide activity at cannabinoid receptors, such as reduced pain sensation (Cravatt, B. F. et al., Proc. Natl. Acad. Sci. U.S.A., 98:9371-9376 (2001)).

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent. Typical modes of administration are described below.

The term “effective amount” means a dosage sufficient to produce a desired result (e.g., reduced appetite, loss of body fat or weight, control weight, reduced craving for or consumption of an appetizing substance). The desired result may comprise a subjective or objective improvement in the recipient of the dosage. With respect to food consumption or food appetite, a subjective improvement may be decreased appetite or craving for food. An objective improvement or measure may be decreased body weight, body fat, or food consumption, or decreased food seeking behavior. Such measures can be directly monitored by measuring the objective or subjective indicia. With respect to an appetizing disorder, a subjective improvement would be a reduced craving or desire for the appetitive substance. An objective improvement would be a decreased consumption or intake of the appetitive substance as determined by reduced tissue levels (e.g., blood, plasma) Or excretion levels (urine, feces) Of the appetitive substance or its metabolites. Such measures can be directly monitored by measuring the objective or subjective indicia.

A “prophylactic treatment” is a treatment administered to a subject who does not exhibit signs of a disease or exhibits only early signs of a disease, wherein treatment is administered for the purpose of decreasing the risk of developing a pathology associated with an unhealthy or undesired appetite or condition such as obesity and the diseases associated with obesity. The compounds of the invention may be given as a prophylactic treatment to prevent undesirable or unwanted weight gain, or unwanted intake of food or other appetative substances such as psychoactive compounds or ethanol.

A “therapeutic treatment” is a treatment administered to a subject who suffers from a pathology (e.g., appetency disorder, obesity) wherein treatment is administered for the purpose of diminishing or eliminating the pathology.

A “combination therapy” refers to a therapy wherein both 1) a cannabinoid receptor antagonist and 2) a PPARα agonist or OEA-like compound or OEA-like agonist or OEA-like appetite reducing compound or FAAH inhibitor are both administered to a subject. The antagonist and agonist may be co-administered or co-formulated for administration. They may be administered separately or at different times. A preferred cannabinoid antagonist is CB1 receptor antagonist (e.g., rimonabant). A preferred OEA-like agonist is clofibrate or a derivative of clofibrate. The combination therapy may be administered for the purpose of treating an appetency disorder, for reducing an appetite for food, reducing body fat or body weight, and/or for modulating lipid metabolism.

The term “to control weight” encompasses the loss of body mass or the reduction of weight gain over time. The methods, compounds and compositions of the present invention are particularly useful for reducing or controlling body fat and body weight in mammals. For instance, the methods, compositions, and compounds of the present invention are helpful in reducing appetite or inducing hypophagia in mammals. The methods, compounds, and compositions are also useful in preventing or mitigating the diseases associated with overweight or obesity by promoting the loss of body fat and body weight. The methods, compounds, and compositions are also useful in treating appetency disorders.

“Synergism” relates to a greater than additive effect resulting from the combination of two compounds. A synergism or synergistic effect of combination therapy with 1) the cannabinoid antagonist and 2) the PPARα agonist or OEA-like agonist, or OEA-like compound, or OEA-like appetite reducing compound or FAAH inhibitor is evident in an effect which is greater than the sum of the effects of the same amount of the cannabinoid antagonist when administered alone (e.g., not as part of a combination therapy) and the same amount of the PPARα agonist or OEA-like agonist, or OEA-like compound, or OEA-like appetite reducing compound or FAAH inhibitor when administered alone. In some embodiments, the effect of the combination therapy is at least 25%, 50%, 100%, or 200% greater than the sum of the effects of the same amount of the cannabinoid antagonist when administered alone (e.g., not as part of a combination therapy) and the same amount of the PPARα agonist or OEA-like agonist, or OEA-like compound, or OEA-like appetite reducing compound or FAAH inhibitor when administered alone. In some embodiments, the synergy is from 50% to 200%, or 200% to 400% greater than the sum of the effects for the individual agents.

I. Compounds of the Invention Generally.

Compounds of the present invention (e.g., OEA-like compounds, OEA-like PPARα modulators, FAAH inhibitors, CB1 cannabinoid receptor inhibitors) may possess asymmetric carbon atoms (optical centers) Or double bonds; the racemates, diastereomers, geometric isomers and individual isomers are all intended to be encompassed within the scope of the present invention.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed by the inventive formulas.

Compounds of the invention include the diastereoisomers of pairs of enantiomers. Diastereomers for example, can be obtained by fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent. Preferred pharmaceutical compositions of the invention contain highly purified forms of the pharmaceutically active enantiomer. In some embodiments, the compositions contain the active enantiomer in an enantiomeric excess (percent active enantiomer minus percent of inactive or less active enantiomer) Of at least 94%, 96%, 98%, 99%.

Alternatively, any enantiomer of an inventive compound may be obtained by stereospecific synthesis using optically pure starting materials or reagents of known configuration.

The compounds of the present invention may have unnatural ratios of atomic isotopes at one or more of their atoms. For example, the compounds may be radiolabeled with isotopes, such as tritium or carbon-14. All isotopic variations of the compounds of the present invention, whether radioactive or not, are within the scope of the present invention.

The instant compounds may be isolated in the form of their pharmaceutically acceptable acid addition salts, such as the salts derived from using inorganic and organic acids. Such acids may include hydrochloric, nitric, sulfuric, phosphoric, formic, acetic, trifluoroacetic, propionic, maleic, succinic, malonic and the like. In addition, certain compounds containing an acidic function can be in the form of their inorganic salt in which the counterion can be selected from sodium, potassium, lithium, calcium, magnesium and the like, as well as from organic bases. The term “pharmaceutically acceptable salts” refers to salts prepared from pharmaceutically acceptable non-toxic bases or acids including inorganic bases or acids and organic bases or acids.

The invention also encompasses prodrugs of the present compounds, which on administration undergo chemical conversion by metabolic processes before becoming active pharmacological substances. In general, such prodrugs will be derivatives of the present compounds that are readily convertible in vivo into a functional compound of the invention. Conventional procedures for the selection and preparation of suitable prodrug derivatives are described, for example, in “Design of Prodrugs”, ed. H. Bundgaard, Elsevier, 1985. The invention also encompasses active metabolites of the present compounds.

As used herein, the term “heteroatom” is meant to include oxygen (O), nitrogen (N), sulfur (S) and silicon (Si).

Where substituent groups are specified by their conventional chemical formulae, written from left to right, they equally encompass the chemically identical substituents which would result from writing the structure from right to left, e.g., —CH₂O— is intended to also recite —OCH₂—.

It has been discovered by the inventors that oleoylethanolamide (OEA), a natural lipid, is a potent body fat reducing and weight control compound when administered to test animals. U.S. Patent Application 60/279,542, filed Mar. 27, 2001, and assigned to the same assignee and herein incorporated by reference in its entirety discloses OEA and OEA-like compounds as agents which can reduce body fat and appetite in mammals. Upon the discovery of the prototype OEA, other fatty acid alkanolamide compounds and homologs were also found to be active. See, U.S. Patent application Ser. No. 10/112,509, filed on Mar. 27, 2002, assigned to the same assignee and herein incorporated by reference in its entirety. See also de Fonseca, et al., Nature, 414:209-212 (2001).

Oleoylethanolamide (OEA) refers to a natural lipid of the following structure:

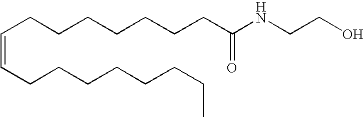

“OEA-like appetite reducing compound(s)” refers to fatty acid ethanolamide(s) and fatty acid alkanolamide compound(s), and its/their homologues or analogs, which can reduce an appetite for, or reduce the consumption of, an appetizing substance upon administration to a test mammal. Such compounds include OEA, elaidoylethanolamide, and palmitoylethanolamide. The appetizing substance may be a food or sugar or other substance. In one embodiment, the appetizing substance is a food. In some embodiments, the OEA-like appetite reducing compound is not an antagonist of the CB1 cannabinoid receptor. In some embodiments, the OEA-like appetite reducing compound is a compound of Formula I or VI, or a pharmaceutically acceptable salt thereof.

“OEA-like compounds” are compounds of formula I which modulate the PPARα receptor as agonists of the receptor. Particularly preferred OEA-like compounds have a selective affinity of at least 5-fold, 10-fold, 50-fold or 100-fold greater for PPARα than for PPARβ or PPARγ. Such preferred OEA-like agonists are particularly preferred if they produce a half-maximal effect on the PPARα receptor under physiological conditions at a concentration of 1 micromolar or less, 100 nanomolar or less, 10 nanomolar or less, or 1 nanomolar or less, or from 1 micromolar to 1.0 nanomolar, or less. Such OEA-like agonists can include, but are not limited to, fatty acid alkanolamides, their homologues and analogues.

“OEA-like PPARα agonists” or “OEA-like agonists” are compounds which specifically bind and act as agonists of the PPARα receptor and/or selectively activate the PPARα receptor. OEA-like agonists include, but are not limited to, fatty acid alkanolamides, fatty acid ethanolamide compounds, and their analogs and homologues which selectively modulate the PPARα receptor. OEA-like agonists have a selective affinity or activation for the PPARα receptor at least 5-fold greater (e.g., having a concentration which produces a half-maximal effect which is at least 5-fold lower) than for either or both PPARβ or PPARγ as measured under comparable bioassay conditions in vivo or in vitro or in any bioassay as described herein. Particularly preferred OEA-like agonists have a selective affinity of at least 5-fold, 10-fold, 50-fold or 100-fold greater for PPARα than for PPARβ or PPARγ. Such preferred OEA-like agonists are particularly preferred if they produce a half-maximal effect on the PPARα receptor under physiological conditions at a concentration of 1 micromolar or less, 100 nanomolar or less, 10 nanomolar or less, or 1 nanomolar or less, or from 1 micromolar to 1.0 nanomolar, or less. Such OEA-like compounds can include, but are not limited to, fatty acid alkanolamides, their homologues and their analogues. Also particularly preferred OEA-like agonists are OEA and compounds of Formula I or Formula VI or VII. In other embodiments, the OEA-like agonist is a specific high affinity agonist of PPARα which is not a fatty acid alkanolamide or a homolog thereof and is not a compound of Formula I or Formula VI. In some embodiments, the OEA-like agonist is selective for the PPARα receptor over a cannabinoid receptor or has negligible cannabinoid receptor affinity or has negligible cannabinoid receptor antagonist activity. OEA-like agonists include compounds whose affinity for the PPARα receptor is at least 5-fold, 10-fold, or 50-fold greater than that for a cannabinoid receptor (e.g., CB1 or CB2 receptor).

An antagonist of the CB1 cannabinoid receptor is a compound which binds to the receptor and lacks any substantial ability to activate the receptor itself. An antagonist can thereby prevent or reduce the functional activation or occupation of the receptor by an agonist such as anandamide when the agonist is present. In some embodiments, the antagonist has an IC₅₀ from about 1 μM to about 1 nM. In other embodiments, the antagonist has an IC₅₀ of from about 0.1 μM to 0.01 μM, 1.0 μM to 0.1 μM, or 0.01 μM to 1 nM. In some embodiments, the antagonist competes with the agonist for binding to a shared binding site on the receptor.

An activation assay is an assay that provides an assessment of the in vivo activation of transcription activators in response to extracellular stimuli. The assessment may be provided by measurement of reporter gene activation, measurement of PPARα mRNA levels, or proliferation of cells transfected with PPARα. It includes assays wherein the activation of PPARα that results from PPARα-RXR heterodimer formation that results from binding of a PPARα subtype specific ligand to PPARα.

An agonist is a ligand of a receptor which activates the receptor or causes signal transduction upon binding to the receptor. OEA is an example of a PPARα receptor agonist.

An antagonist is a ligand of a receptor which binds to the receptor but does not appreciably activate the receptor or appreciably cause signal transduction. An antagonist may block the ability of an agonist to bind and activate a receptor or otherwise reduce the activity of the receptor under physiological conditions.

A binding assay is an assay that provides an assessment of ligand binding to a receptor (e.g., PPARα, PPARβ, or PPARγ receptors). For instance, the assessment may be provided by measurement of displacement of radioactively labeled PPARα ligand, of electrophoretic mobility shifts, measurement of immunoprecipitation of PPARα, PPARβ, or PPARγ to antibodies. The assessment may be accomplished through high throughput screening. A “specific” binder or binding of PPARα refers to a compound or binding interaction that has at least 5 fold greater affinity (e.g., as measured by EC50's or IC50's) for PPARα than for PPARγ or for PPARβ. Binding is not determinative that a ligand is an agonist or an antagonist.

A peroxisome proliferator activated receptor (PPAR) is a member of a family of nuclear receptors, distinguished in α, β, and γ subtypes as described herein.

A specific or selective PPAR activator is a compound that preferentially binds and activates one PPAR subtype over another. For example, a specific activator of PPARα is OEA.

A specific or selective binder is a compound that preferentially binds one PPAR subtype over another. For example, a specific binder of PPARα is OEA.

“Alkanol,” as used herein refers to a saturated or unsaturated, substituted or unsubstituted, branched or straight alkyl group having a hydroxyl substituent, or a substituent derivable from a hydroxyl moiety, e.g., ether, ester. The alkanol is preferably also substituted with a nitrogen-, sulfur-, or oxygen-bearing substituent that is included in bond Z (Formula I), between the “fatty acid” and the alkanol.

“Fatty acid,” as used herein, refers to a saturated or unsaturated substituted or unsubstituted, branched or straight alkyl group having a carboxyl substituent. Preferred fatty acids are C₄-C₂₂ acids. Fatty acid also encompasses species in which the carboxyl substituent is replaced with a —CH₂— moiety.

The term “alkyl,” by itself or as part of another substituent, means, unless otherwise stated, a straight or branched chain, or acylic or cyclic, chiral or achiral, hydrocarbon radical, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one (alkenyl) Or more double bonds (alkadienyl) Or triple bonds (alkynyl). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

In the formulas herein, “Me” represents the methyl group.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 or 2 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) Can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

A. Fatty Acid Alkanolamide Compounds, Homologs, and Analogs.

The OEA-like appetite reducing compounds according to the invention include fatty acid alkanolamide compounds, their homologs, and analogs, including particularly, compounds of Formulae I-VI below. Such compounds may be identified and defined in terms of either an ability to cause reduced appetite, food intake, and/or body weight or body fat upon administration to test animals in vivo. In some embodiments, these compounds are not significant antagonists of the CB1 cannabinoid receptor, particularly, with respect to the administered therapeutic doses used or the therapeutic concentrations required for activity. A compound is not a not significant or substantial antagonist of the CB1 cannabinoid receptor if 1) its effects on appetite or the reduction of food intake are not directly and primarily mediated by the binding of the compound to the CB1 receptor.

1. Fatty Acid Alkanolamide Compounds, Homologs, and Analogs for Use According to the Invention.

OEA-like compounds, OEA-like agonists and OEA-like appetite reducing compounds encompass, but are not limited to, a variety of fatty acid alkanolamides, homologs and analogs which are PPARα agonists. These fatty acid alkanolamides, homologs and analogs include compounds having the following general formula:

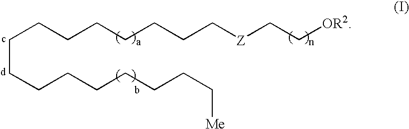

In this formula, n is any number from 0, 1, 2, 3, 4 or 5 and the sum of a and b can be any number from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of unsubstituted or substituted, straight or branched alkyl, hydrogen, substituted or unsubstituted C₁-C₆ alkyl, substituted or unsubstituted lower (C₂-C₆) acyl, (C₁-C₆) homoalkyl, and aryl. Up to eight hydrogen atoms of the compound may also be substituted by methyl group or by a double bonds linking adjacent carbons. In addition, the molecular bond between carbons c and d may be unsaturated or saturated. In some embodiments, the fatty acid alkanolamide or ethanolamide of the above formula is a naturally occurring compound. In some preferred embodiments, the alkyl subsitutents are each homoalkyl. In some embodiments where R^{o} or R² is an acyl group, the acyl groups may be that of the propanoic, ethanoic, 2,2-dimethylpropanoic or butanoic acid and attached via an ester linkage as R² or an amide linkage as R^{o}. In some embodiments, a H atom attached to a carbon atom of a compound of the above formula is replaced with a halogen atom, preferably a Cl atom or a F atom.

OEA-like compounds, OEA-like agonists, and OEA-like appetite reducing compounds of the invention also include compounds of the following formula:

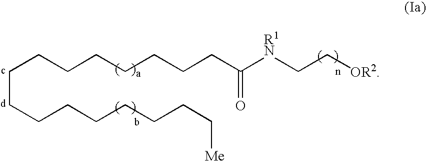

In one embodiment, the compounds of Formula Ia have n from 0 to 5; and a sum of a and b that is from 0 to 4; and members R¹ and R² independently selected from the group consisting of hydrogen, substituted or unsubstituted C₁-C₆ alkyl, lower substituted or unsubstituted (C₁-C₆) acyl, homoalkyl, and substituted or unsubstituted aryl. In this embodiment, up to eight hydrogen atoms of the fatty acid portion and alkanolamine (e.g., ethanolamine) portion of compounds of the above formula may also be substituted by a methyl group or replaced by a double bond between adjacent carbons. In addition, the molecular bond between carbons c and d may be unsaturated or saturated. In some embodiments where R¹ or R² is an acyl group, the acyl groups may be that of the propanoic, ethanoic, 2,2-dimethylpropanoic or butanoic acid and attached via an ester linkage as R² or an amide linkage as R¹. In some embodiments, a H atom attached to a carbon atom of a compound of the above formula is replaced with a halogen atom, preferably a Cl atom or a F atom.

In another embodiment, the above compounds particularly include those in which the fatty acid moiety comprises oleic acid, elaidic acid, or palmitic acid. Such compounds include oleoylethanolamide, elaidoylethanolamide and palmitoylethanolamide.

In still another embodiment, the compounds of Formula Ia have n from 1 to 3; and a sum of a and b that is from 1 to 3; and members R¹ and R² independently selected from the group consisting of hydrogen, substituted or unsubstituted C₁-C₆ alkyl, and lower substituted or unsubstituted (C₂-C₆) acyl. In this embodiment, up to four hydrogen atoms of the fatty acid portion and alkanolamine (e.g., ethanolamine) portion of compounds of the above formula may also be replaced by a methyl or replaced by a double bond joining adjacent carbons. In addition, the molecular bond between carbons c and d may be unsaturated or saturated. In a further embodiment, the molecular bond between carbons c and d is unsaturated and no other hydrogen atoms are substituted. In a still further embodiment thereof, the members R¹ and R² are independently selected from the group consisting of hydrogen, substituted or unsubstituted C₁-C₃ alkyl, and substituted or unsubstituted lower (C₂-C₅) acyl.

Exemplary compounds provide mono-methyl substituted compounds, including ethanolamides, of Formula Ia. Such compounds include:

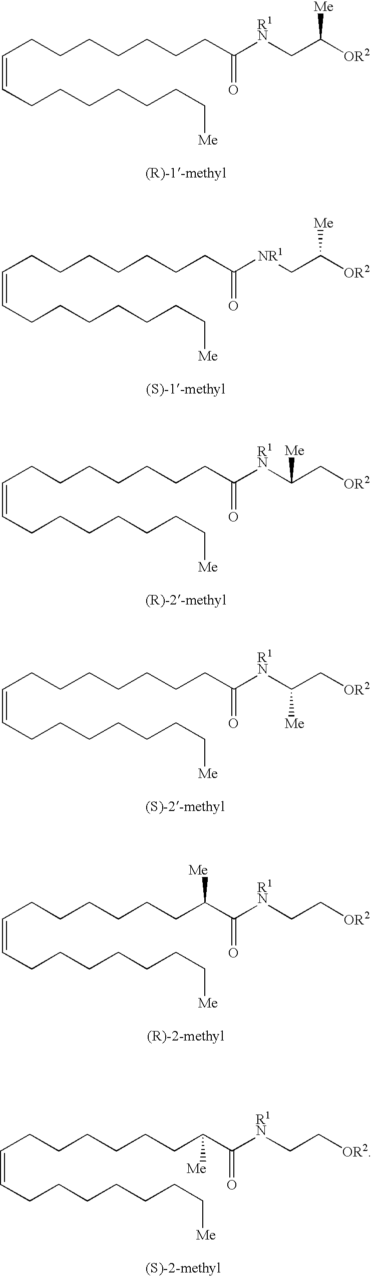

The methyl substituted compounds of the above formula include particularly those compounds where R¹ and R² are both H: (R)-1′-methyloleoylethanolamide, S-(1′)-methyloleoylethanolamide, (R)-2′-methyloleoylethanolamide, (S)-2′-methyloleoylethanolamide, (R)-2-methyloleoylethanolamide(hydroxyethyl-Z-2-(R)-methyloctadec-9-enamide), and (S)-2-methyloleoylethanolamide(hydroxyethyl-Z-2-(S)-methyloctadec-9-enamide).

B. Reverse OEA-Like Compounds.

OEA-like compounds, OEA-like agonists, and OEA-like appetite reducing compounds of the invention also include a variety of analogs of OEA. These compounds include reverse OEA compounds of the general formula:

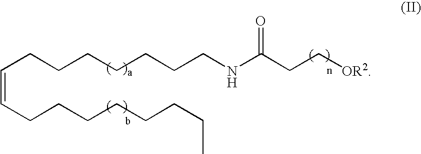

In some embodiments, the invention provides compounds of Formula II. In still other embodiments, the compounds of Formula II have n from 1 to 5, and a sum of a and b from 0 to 4. In such embodiments, the member R² is selected from the group consisting of hydrogen, substituted or unsubstituted C₁-C₆ alkyl, substituted or unsubstituted lower (C₂-C₆) acyl, (C₁-C₆) homoalkyl, and aryl. In addition, up to four hydrogen atoms of either or both the alkylamine portion and hydroxycarboxylic acid portion (e.g., hydroxyalkanoic acid portion) Of compounds of the above formula may also be substituted by a methyl group or a double bond joining adjacent atoms.

In some embodiments, the compounds of formula II include those compounds where the hydroxycarboxylic acid portion is 3-hydroxypropanoic acid where R² is H, and compounds where a and b are each 1, and compounds where n is 1.

One embodiment of a compound according to Formula II is

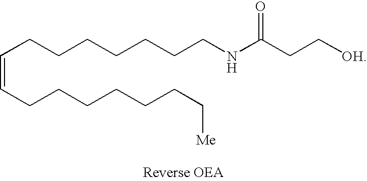

In another embodiment, the compounds of Formula II have n from 1 to 5 and a sum of a and b from 1 to 3. In this embodiment, the member R² is selected from the group consisting of hydrogen, substituted or unsubstituted C₁-C₆ alkyl, and substituted or unsubstituted lower (C₂-C₆) acyl. In addition, up to four hydrogen atoms of either or both the alkylamine portion and hydroxyalkylcarboxyl portion of compounds of the above formula may also be replaced by a methyl group or by a double bond adjoining adjacent atoms.

C. Oleoylalkanediol Monoester Compounds.

OEA-like compounds, OEA-like agonist, and OEA-like appetite reducing compounds of the invention also include oleoylalkanediol monoesters of the general formula:

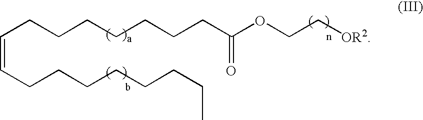

In some embodiments, the compounds of Formula III, have n from 1 to 5; and the sum of a and b from 0 to 4. The member R² is selected from the group consisting of hydrogen, substituted or unsubstituted C₁-C₆ alkyl, lower (C₂-C₆) acyl, C₁-C₆ homoalkyl, and aryl. Up to four hydrogen atoms of either or both the fatty acid portion and alkanediol (e.g., ethanol ethanediol or ethylene glycol) portion of compounds of the above formula may also be replaced by a methyl group or a double bond joining adjacent carbons.

In some embodiments, the compounds of Formula III, have n from 1 to 3; and the sum of a and b from 1 to 3. The member R² is selected from the group consisting of hydrogen, substituted or unsubstituted C₁-C₆ alkyl, and substituted or unsubstituted lower (C₁-C₆) acyl. Up to four hydrogen atoms of the fatty acid portion and alkanediol (e.g., ethanediol or ethylene glycol) portion of compounds of the above formula may also be substituted by methyl or a double bond.

Compounds of Formula III include those compounds where R² is H, compounds where a and b are each 1, and compounds where n is 1. Examples of compounds according to Formula III include a compound (oleoyl 2-hydroxyethyl ester (Z-2-hydroxyethyl octadec-9-enoate) Of the following formula

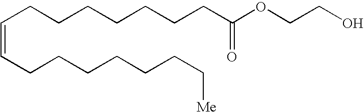

Compounds of Formula III also include mono-methyl substituted oleoyl ethanediol esters such as the (R or S)— Z-2-(1,2-dihydroxypropyl octadec-9-eneoate; the (R or S)-1′-Z-1-(1,2-dihydroxypropyl octadec-9-eneoate; and the (R or S))-Z-2-hydroxyethyl 2-methyloctadec-9-eneoate; respectively:

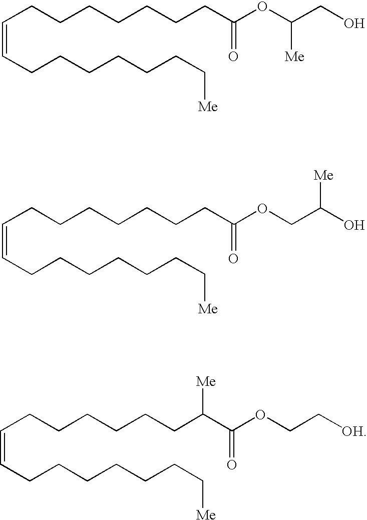

D. Oleoyl Alkanol Ethers

OEA-like compounds, OEA-like agonists, and OEA-like appetite reducing compounds of the invention also include ethers of a fatty alcohol (e.g., oleyl alcohol) and an alkanediol according to the general formula:

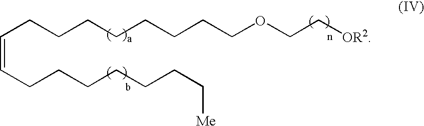

In some embodiments, the compounds of Formula IV, have an n from 1 to 5 and a sum of a and b that can be from 0 to 4. The member R² is selected from the group consisting of hydrogen, substituted or unsubstituted C₁₋₆ alkyl, substituted or unsubstituted lower (C₂-C₆) acyl, C₁-C₆ homoalkyl, and substituted and unsubstituted aryl. Up to four hydrogen atoms of either or both the fatty alcohol portion and alkanediol (e.g., ethanediol) portion of compounds of the above formula may also be replaced by a methyl group or a double bond joining adjacent carbons.

In other embodiments, the compounds of Formula IV, have n from 1 to 3; and the sum of a and b can be from 1 to 3. The member R² is selected from the group consisting of hydrogen, substituted or unsubstituted C₁-C₆ alkyl, and substituted or unsubstituted lower (C₂-C₆) acyl. Up to four hydrogen atoms of either or both the fatty alcohol portion and alkanediol (e.g., ethanediol) portion of compounds of the above formula may also be replaced by a methyl group or by a double bond joining adjacent carbons.

Compounds of Formula IV include those compounds where R² is H, compounds where a and b are each 1, and compounds where n is 1. Examples of compounds according to Formula IV include (R or S) Compounds of the following formula:

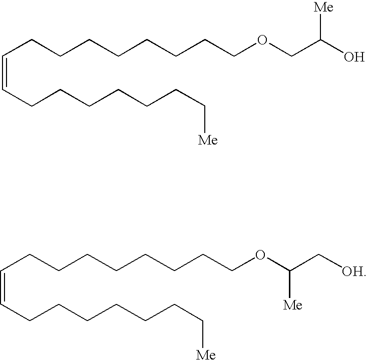

E. Fatty Acid Alkanolamide Analogs Having Polar Head Variants.

OEA-like compounds, OEA-like agonists, and OEA-like appetite reducing compounds of the invention include compounds having a variety of polar head analogs of OEA. These compounds include compounds having a fatty acid moiety of the general formula:

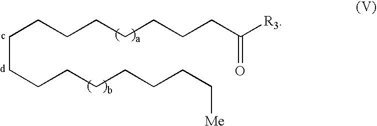

In some embodiments, the compounds of Formula V have a sum of a and b that can be from 0 to 4. In other embodiments, the sum of a and b is from 1 to 3. In these embodiments, up to four hydrogen atoms of the compounds of the above formula may also be substituted by methyl or a double bond. In addition, the molecular bond between carbons c and d may be unsaturated or saturated. A particularly preferred embodiment is that of the oleic acid fatty acid moiety:

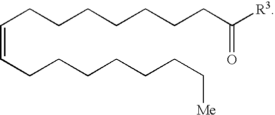

The R³ group of the above structures may be selected from any of the following:

HO—(CH₂)ₙ—NH— wherein z is from 1 to 5, and the alkyl portion thereof is an unbranched methylene chain. For example:

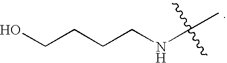

H₂N—(CH₂)ₙ—NH— wherein z is from 1 or 2 to 5, and the alkyl portion thereof is an unbranched methylene chain. For example:

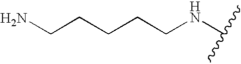

HO—(CH₂)ₓ—NH— wherein x is from 1 to 8, and the alkylene portion thereof may be branched or cyclic. For example,

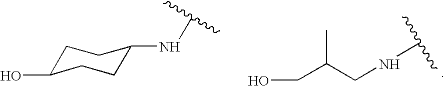

Additional polar head groups for R³ include, for instance, compounds having furan, dihydrofuran and tetrahydrofuran functional groups: 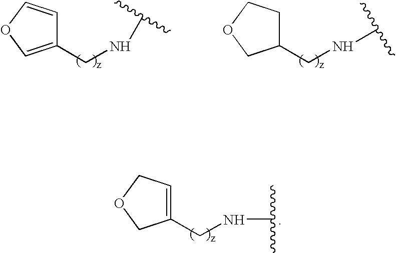
In the above structures, z can be from 1 to 5.

Such compounds of the invention include, for instance, those having R³ polar head groups based upon pyrole, pyrrolidine, and pyrroline rings: 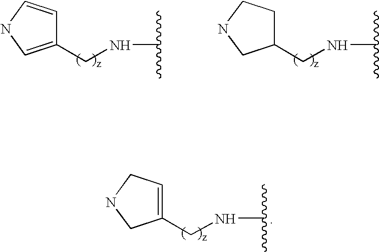
In the compounds of the above structures, z can be from 1 to 5.

Other polar head groups include a variety of imidazole and oxazoles, for example: 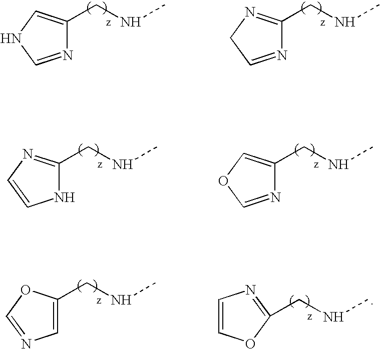
In the compounds of the above structures, z can be from 1 to 5.

Other embodiments have oxazolpyridine polar head groups:

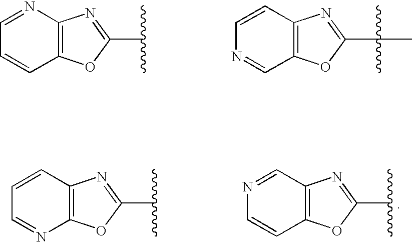

F. Fatty Acid Alkanolamide Analogs Having Apolar Tail Variants.

OEA-like compounds, OEA-like agonists, and OEA-like appetite reducing compounds of the invention include a variety of alkanolamide and ethanolamide compounds having a variety of flexible apolar tails. These compounds include compounds of the following formulas in which R represents an ethanolamine moiety, an alkanolamine moiety, or a stable analog thereof. In the case of ethanolamine, the ethanolamine moiety is attached preferably via the ethanolamine nitrogen rather than the ethanolamine oxygen. 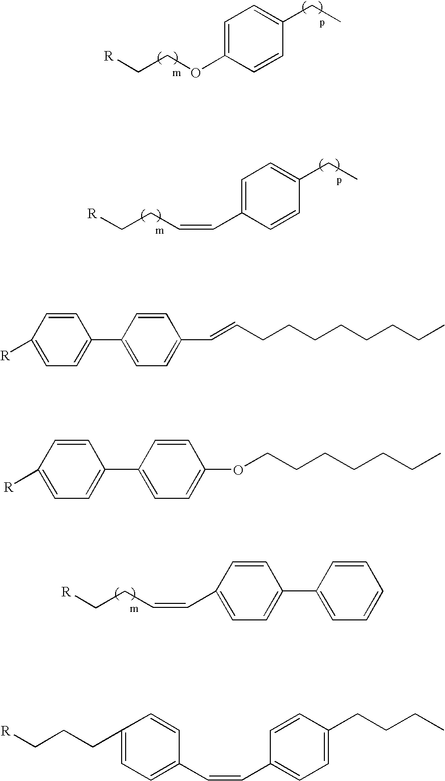
In the above structures, m is from 1 to 9 and p is independently from 1 to 5.

In another embodiment, the compound is:

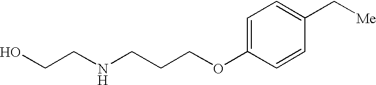

A compound of another embodiment is an ethanolamine analog with an apolar tail of the following structural formula:

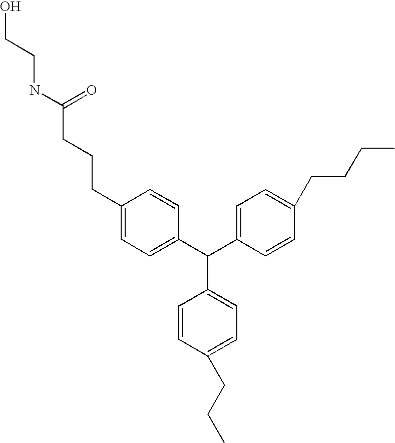

OEA-like compounds, OEA-like appetite reducing compounds of the invention of the invention include those disclosed in U.S. Patent application Ser. No. 10/112,509 filed Mar., 27, 2002, assigned to the same assignee as the present application, which is incorporated herein by reference. In other embodiments, the fatty acid moiety of the fatty acid alkanolamide or ethanolamide compound, homologue, or analog may be saturated or unsaturated, and if unsaturated may be monounsaturated or polyunsaturated.

In some embodiments, the fatty acid moiety of the fatty acid alkanolamide compound, homologue, or analog is a fatty acid selected from the group consisting of oleic acid, palmitic acid, elaidic acid, palmitoleic acid, linoleic acid, α-linolenic acid, and γ-linolenic acid. In certain embodiments, the fatty acid moieties have from twelve to 20 carbon atoms.

Other embodiments are provided by varying the hydroxyalkylamide moiety of the fatty acid amide compound, homologue or analog. These embodiments include the introduction of a substituted or unsubstituted lower (C₁-C₃) alkyl group on the hydroxyl group of an alkanolamide or ethanolamide moiety so as to form the corresponding lower alkyl ether. In another embodiment, the hydroxy group of the alkanolamide or ethanolamide moiety is bound to a carboxylate group of a C₂ to C₆ substituted or unsubstituted alkyl carboxylic acid to form the corresponding ester of the fatty acid ethanolamide. Such embodiments include fatty acid alkanolamide and fatty acid ethanolamides in ester linkage to organic carboxylic acids such as acetic acid, propionic acid, and butanoic acid. In one embodiment, the fatty acid alkanolamide is oleoylalkanolamide. In a further embodiment, the fatty acid alkanolamide is oleoylethanolamide.

In still another embodiment, the fatty acid ethanolamide compound, homologue, or analog further comprises a substituted or unsubstituted lower alkyl (C₁-C₃) group covalently bound to the nitrogen atom of the fatty acid ethanolamide.

In still another embodiment, the OEA-like compound, agonist, or appetite reducing compound for use according to the invention is fatty acid alkanolamide compound or homologue satisfying the following formula VI:

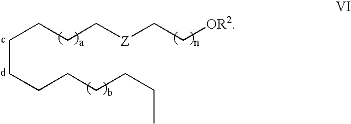

In this formula, n is any number from 0 to 5 and the sum of a and b can be any number from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of substituted or unsubstituted alkyl, hydrogen, substituted or unsubstituted C₁-C₆ alkyl, substituted or unsubstituted lower (C₁-C₆) acyl, homoalkyl, and aryl. Up to six hydrogen atoms of the compound may also be substituted by methyl group or a double bond. In addition, the molecular bond between carbons c and d may be unsaturated or saturated. In some embodiments, the fatty acid ethanolamide of the above formula is a naturally occurring compound. In some preferred embodiments, the alkyl subsitutents are each homoalkyl, or its pharmaceutically acceptable salt. Further embodiments of the compounds of Formula VI have substituents as set forth for compounds of Formula I above. In some embodiments, a H atom attached to a carbon atom of a compound of the above formula is replaced with a halogen atom, preferably a Cl atom or a F atom.

G. Synthesis of Fatty Acid Alkanolamides.

Compounds useful in practicing the present invention can be readily synthesized and purified using methods recognized in the art. In an exemplary synthetic scheme (Scheme 1), a carboxylic acid and an aminoalcohol (or an O-protected derivative thereof) are reacted in a the presence of a dehydrating agent, e.g., dicyclohexylcarbodiimide, in an appropriate solvent. The fatty acid alkanol amide is isolated by methods such as extraction, crystallization, precipitation, chromatography and the like. If the final product is the O-protected adduct, it is deprotected, typically by an art-recognized method, to afford a fatty acid adduct having a free hydroxyl group.

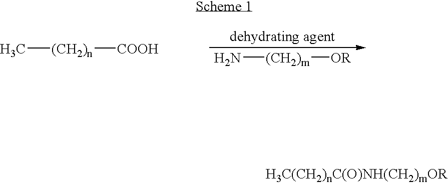

Those of skill in the art will recognize that many variants on the scheme set forth above are available. For example, an activated derivative, e.g, acyl halide, active ester, of the acid can be used. Similarly, a glycol (preferably mono O-protected) Can be substituted for the amino alcohol, resulting in an ester linkage between the two constituents of the molecule.

Reverse esters and reverse amides can also be readily synthesized by art-recognized methods. For example, a hydroxycarboxylic acid is reacted with an amine or hydroxy derivative of a long chain alkyl (i.e., C₄-C₂₂) in the presence of a dehydrating agent. In certain reaction pathways, it is desirable to protect the hydroxyl moiety of the hydroxycarboxylic acid.

Ethers and mercaptans can be prepared by methods well-known to those of skill in the art, e.g., Williamson synthesis. For example, a long chain alkyl alcohol or thiol is deprotonated by a base, e.g, NaH, and a reactive alcohol derivative, e.g., a halo, tosyl, mesyl alcohol, or a protected derivative thereof is reacted with the resulting anion to form the ester or mercaptan.

The above-recited methods and variations thereof can be found in, for example, RECENT DEVELOPMENTS IN THE Synthesis OF FATTY ACID DERIVATIVES, Knothe G, ed., Amer. Oil Chemists Society 1999; COMPREHENSIVE NATURAL PRODUCTS CHEMISTRY AND OTHER SECONDARY METABOLITES INCLUDING FATTY ACIDS AND THEIR DERIVATIVES, Nakanishi K, ed., Pergamon Press, 1999; ORGANIC SYNTHESIS COLLECTED VOLUMES I-V, John Wiley and Sons; COMPENDIUM OF ORGANIC SYNTHETIC METHODS, Volumes 1-6, Wiley Interscience 1984; ORGANIC FUNCTIONAL GROUP PREPARATION, Volumes I-III, Academic Press Ltd. 1983; Greene T, PROTECTING GROUPS IN ORGANIC SYNTHESIS, 2d ed., Wiley Interscience 1991.

H. OEA-Like PPARα Agonists which are not OEA-Like Compounds.

In addition, OEA-like agonists need not be an OEA-like compound (e.g., OEA, fatty acid amide or homolog thereof). In some embodiments, the OEA-like agonist is a compound such as taught in U.S. Pat. No. 6,200,998 (hereby incorporated by reference) that are PPARα activators. This reference teaches PPAR agonist compounds of the general formula:

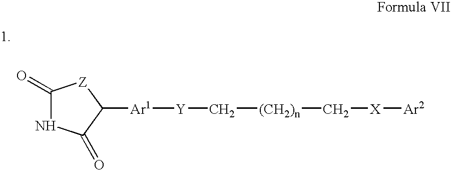

In the above formula, Ar¹ is (1) arylene or (2) heteroarylene, wherein arylene and heteroarylene are optionally substituted with from 1 to 4 groups selected from R^{a} (defined below); Ar² is (1) Ortho-substituted aryl or (2) Ortho-substituted heteroaryl, wherein said ortho substituent is selected from R (defined below); and aryl and heteroaryl are optionally further substituted with from 1-4 groups independently selected from R^{a}; X and Y are independently O, S, N—R^{b} (defined below), or CH₂; Z is O or S; n is 0 to 3; R is (1) C₃₋₁₀ alkyl optionally substituted with 1-4 groups selected from halo and C₃₋₆ cycloalkyl, (2) C₃₋₁₀ alkenyl, or (3) C₃₋₈ cycloalkyl; R^{a} is (1) C₁₋₁₅ alkanoyl, (2) C₁₋₁₅ alkyl, (3) C₂₋₁₅ alkenyl, (4) C₂₋₁₅ alkynyl, (5) halo, (6) OR^{b}, (7) aryl, or (8) heteroaryl, wherein said alkyl, alkenyl, alkynyl, and alkanoyl are optionally substituted with from 1-5 groups selected from R^{c} (defined below), and said aryl and heteroaryl optionally substituted with 1 to 5 groups selected from R^{d} (defined below); Rb is (1) hydrogen, (2) C₁₋₁₀ alkyl, (3) C₂₋₁₀ alkenyl, (4) C₂₋₁₀ alkynyl, (5) aryl, (6) heteroaryl, (7) aryl C₁₋₁₅ alkyl, (8) heteroaryl C₁₋₁₅ alkyl, (9) C₁₋₁₅ alkanoyl, (10) C₃₋₈ cycloalkyl, wherein alkyl, alkenyl, alkynyl are optionally substituted with one to four substituents independently selected from R^{c}, and cycloalkyl, aryl and heteroaryl are optionally substituted with one to four substituents independently selected from R^{d}; or R^{c} is (1) halo, (2) aryl, (3) heteroaryl, (4) CN, (5) NO₂, (6) OR^{f}; (7) S(O)ₘR^{f}, m=0, 1 or 2, provided that R^{f} (defined below) is not H when m is 1 or 2; (8) NR^{f}R^{f,} (9) NR^{f}COR^{f}, (10) NR^{f}CO₂R^{f}, (11) NR^{f}CON(R^{f})₂, (12) NR^{f}SO₂R^{f}, provided that R^{f} is not H, (13) COR^{f}, (14) CO₂R^{f}, (15) CON(R^{f})₂, (16) SO₂N(R^{f})₂, (17) OCON(R^{f})₂, or (18) C₃₋₈ cycloalkyl, wherein said cycloalkyl, aryl and heteroaryl are optionally substituted with 1 to 3 groups of halo or C₁₋₆ alkyl; R^{d} is (1) a group selected from R^{c}, (2) C₁₋₁₀ alkyl, (3) C₂₋₁₀ alkenyl, (4) C₂₋₁₀ alkynyl, (5) aryl C₁₋₁₀ alkyl, or (6) heteroaryl C₁₋₁₀ alkyl, wherein alkyl, alkenyl, alkynyl, aryl, heteroaryl are optionally substituted with a group independently selected from R^{e}; R^{e} is (1) halogen, (2) amino, (3) Carboxy, (4) C₁₋₄ alkyl, (5) C₁₋₄ alkoxy, (6) hydroxy, (7) aryl, (8) aryl C₁₋₄ alkyl, or (9) aryloxy; R^{f} is (1) hydrogen, (2) C₁₋₁₀ alkyl, (3) C₂₋₁₀ alkenyl, (4) C₂₋₁₀ alkynyl, (5) aryl, (6) heteroaryl, (7) aryl C₁₋₁₅ alkyl, (8) heteroaryl C₁₋₁₅ alkyl, (9) C₁₋₁₅ alkanoyl, (10) C₃₋₈ cycloalkyl; wherein alkyl, alkenyl, alkynyl, aryl, heteroaryl, alkanoyl and cycloalkyl are optionally substituted with one to four groups selected from R^{e}.

Also preferred are those PPARα specific activators as taught in U.S. Pat. No. 5,859,051. These activators have the following general formula as set forth in the U.S. Pat. No. 5,589,051:

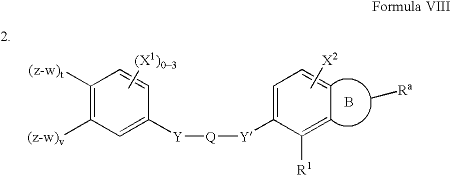

In the embodiments according to Formula VIII, R¹ is selected from a group consisting of: H, C₁₋₁₅ alkyl, C₂₋₁₅ alkenyl, C₂₋₁₅ alkynyl and C₃₋₁₀ cycloalkyl, said alkyl, alkenyl, alkynyl, and cycloalkyl optionally substituted with 1 to 3 groups of R^{a} (defined below); R³ is selected from a group consisting of: H, NHR¹, NHacyl, C₁₋₁₅ alkyl, C₃₋₁₀ cycloalkyl, C₂₋₁₅ alkenyl, C₁₋₁₅ alkoxy, CO₂ alkyl, OH, C₂₋₁₅ alkynyl, C₅₋₁₀ aryl, C₅₋₁₀ heteroaryl said alkyl, cycloalkyl, alkenyl, alkynyl, aryl and heteroaryl optionally substituted with 1 to 3 groups of R^{a}; (Z-W—) is Z-CR⁶R⁷—, Z-CH.═CH—, or:

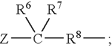

R⁸ is selected from the group consisting of CR⁶R⁷, O, NR⁶, and S(O)ₚ; R⁶ and R⁷ are independently selected from the group consisting of H, C₁₋₆ alkyl; B is selected from the group consisting of: 1) a 5 or 6 membered heterocycle containing 0 to 2 double bonds, and 1 heteroatom selected from the group consisting of O, S and N, the heteroatom being substituted at any position on the five or six membered heterocycle, the heterocycle being optionally unsubstituted or substituted with 1 to 3 groups of R^{a}; 2) a 5 or 6 membered carbocycle containing 0 to 2 double bonds, the carbocycle optionally unsubstituted or substituted with 1 to 3 groups of R^{a} at any position on the five or six membered carbocycle; and 3) a 5 or 6 membered heterocycle containing 0 to 2 double bonds, and 3 heteroatoms selected from the group consisting of O, N, and S, which are substituted at any position on the five or six membered heterocycle, the heterocycle being optionally unsubstituted or substituted with 1 to 3 groups of R^{a}; X¹ and X² are independently selected from a group consisting of: H, OH, C₁₋₁₅ alkyl, C₂₋₁₅ alkenyl, C₂₋₁₅ alkynyl, halo, OR³, ORCF₃, C₅₋₁₀ aryl, C₅₋₁₀ aralkyl, C₅₋₁₀ heteroaryl and C₁₋₁₀ acyl, said alkyl, alkenyl, alkynyl, aryl and heteroaryl optionally substituted with 1 to 3 groups of R^{a}; R^{a} represents a member selected from the group consisting of: halo, acyl, aryl, heteroaryl, CF₃, OCF₃, —O—, CN, NO₂, R³, OR³; SR³, ═N(OR), S(O)R³, SO₂R³, NR³R³, NR³COR³, NR³CO₂R³, NR³CON(R³)₂, NR³SO₂R³, COR³, CO₂R³, CON(R³)₂, SO₂N(R³)₂, OCON(R³)₂ said aryl and heteroaryl optionally substituted with 1 to 3 groups of halo or C₁₋₆ alkyl; Y is selected from the group consisting of: S(O)ₚ, —CH₂—, —C(O)—, —C(O)NH—, —NR—, —O—, —SO₂NH—, —NHSO₂; Y¹ is selected from the group consisting of: O and C; Z is selected from the group consisting of: CO₂R³, R³CO₂R³, CONHSO₂Me, CONHSO₂, CONH₂ and 5-(1H-tetrazole); t and v are independently 0 or 1 such that t+v=1 Q is a saturated or unsaturated straight chain hydrocarbon containing 2-4 carbon atoms and p is 0-2 with the proviso when Z is CO₂R³ and B is a 5 membered heterocycle consisting of 0, R³ does not represent methyl.

Additional compounds suitable for practicing the inventive methods include compounds taught in U.S. Pat. No. 5,847,008, U.S. Pat. No. 6,090,836 and U.S. Pat. No. 6,090,839, U.S. Pat. No. 6,160,000 each of which is herein incorporated by reference in its entirety to the extent not inconsistent with the present disclosure.

Additionally, a variety of suitable PPAR agonists and activators for screening are taught in U.S. Pat. No. 6,274,608. Aryl and heteroaryl acetic acid and oxyacetic acid compounds are taught for instance in U.S. Pat. No. 6,160,000; substituted 5-aryl-2,4-thiazolidinediones are taught in U.S. Pat. No. 6,200,998; other compounds including PPARα-specific polyunsaturated fatty acids and eicosanoids are known as described in Forman, B M, Chen, J, and Evans R M, PNAS 94:4312-4317 and PCT Patent Publication No. WO 97/36579, published Oct. 9, 1997). The compositions of these publications, which are each herein incorporated by reference in their entirety to the extent not inconsistent with the present disclosure can be screened by the methods provide below to provide the PPARα specific agonists of the invention which are useful, for instance, in reducing body fat, and body weight, modulating fat catabolism, and reducing appetite according to the present disclosure.

In some embodiments, the PPARα agonist is clofibrate or a derivative of clofibrate. Such compounds include, but are not limited to, clofibrate (i.e., 2-(4-chlorophenoxy)-2-methylpropanoic acid, ethyl ester); fenofibrate, (1-methylethyl 2-[4-(4-chlorobenzoyl)phenoxy]-2-methylpropanoate; 2-[4-(4-chlorobenzoyl)phenoxy]-2-methylpropanoic acid, 1-methylethyl ester); bezafibrate (2-[4-[2-[(4-chlorobenzoyl)amino]-ethyl]phenoxy]-2-methyl-propanoic acid, gemfibrozil: 5-(2,5-dimethylphenoxy)-2,2-dimethylpentanoic acid and ciprofibrate.

Other PPARα agonists suitable for use in the methods and compostions of the invention are clofibrate derivative compounds of the following formula or their pharmaceutically acceptable salts: 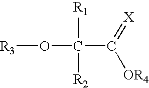
wherein R₁ and R₂ may be the same or different and are each a hydrogen atom or a substituted or unsubstituted alkyl, alkoxy, or phenoxy group, R₃ is a substituted or unsubstituted aryl group phenyl group and X is hydrogen (2H) Or oxygen, and R₄ is H or alkyl. In one embodiment, the R₃ aryl group is substituted or unsubstituted phenyl, preferably monosubstituted. In another embodiment, X is O and R₃ is a mono-, di- or tri-substituted phenyl group, bearing one, two or three identical or different substituents for an aryl group and R₁ and R₂ are each, independently, a hydrogen atom or an alkyl group. In a further embodiment, R₃ is a is a mono-, di- or tri-substituted phenyl group, bearing one, two or three identical or different substituents which are one or more of the following, namely halogen atoms and alkyl, alkoxy, aryl, heteroaryl, or hydroxy groups, and R₁ and R₂ are each, independently, a hydrogen atom or an alkyl group, and R₄ is H or alkyl.

Each of the above Patents cited in this section are incorporated by reference herein with particular reference to the compounds and compositions they disclose.

II. Bioassay Methods for Assessing the Effects of Compounds, Compositions, and Combination Therapies on Appetite(s), Body Fat Reduction, Body Weight, and Lipid Metabolism.

In whole animal bioassays, administration of an appropriate amount of the compound(s) Or compositions or combination therapy for possible use according to the invention may be by any means known in the art such as, for example, topical, oral, rectal, parenteral such as, for example, intraperitoneal, intravenous, subcutaneous, subdermal, intranasal, or intramuscular. Preferably administration may be intraperitoneal or oral. An appropriate effective amount of the candidate compound may be determined empirically as is known in the art. For example, with respect to food consumption or reductions in body weight or body fat, an appropriate effective amount may be an amount sufficient to effect a loss of body fat or a loss in body weight or reduction in food consumption in the animal over time. The candidate compound(s) and therapies can be administered as often as required to effect a loss of body fat or loss in body weight, for example, hourly, every six, eight, twelve, or eighteen hours, daily, or weekly.

Formulations suitable for oral administration include, but are not limited to, (a) liquid solutions, such as an effective amount of the candidate compound(s) suspended in diluents, such as water, saline or PEG 400; (b) Capsules, sachets or tablets, each containing a predetermined amount of the active ingredient, as liquids, solids, granules or gelatin; (c) suspensions in an appropriate liquid; and (d) suitable emulsions. Tablet forms include, but are not limited to, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Injection solutions and suspensions can be prepared from sterile powders, granules, and tablets of the kind previously described. Formulations suitable for parenteral administration, include, but are not limited to, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include, but are not limited to, suspending agents, solubilizers, thickening agents, stabilizers, and preservatives.

The dose(s) administered to the animal are sufficient to determine if the compounds, compositions or combination therapy has a desired effect, for example, an appetite, body weight, body fat, and/or fatty acid oxidation over time. Such dose(s) Can be determined according to the efficacy of the particular candidate compound(s) employed and the condition of the animal, as well as the body weight or surface area of the animal. The size of the dose(s) also will be determined by the existence, nature, and extent of any adverse side-effects that accompany the administration of a candidate compound(s); the LD50 of the candidate compound(s); and the side-effects of the candidate compound(s) at various concentrations. Depending upon the compound(s) and the above factors, for instance, the initial test dosage(s) may range, for example, from 0.1-50 mg per kg, preferably 1-25 mg per kg, most preferably 1-20 mg per kg body weight for each of the compound(s). The determination of dose response relationships is well known to one of ordinary skill in the art.

Test animals subjects can be, for example, obese or normal mammals (e.g., humans, primates, guinea pigs, rats, mice, or rabbits). Suitable rats include, but are not limited to, Zucker rats. Suitable mice include, but are not limited to, for example, ALS/LtJ, C3.SW-H-2b/SnJ, (NON/LtJ x NZO/HIJ)F1, NZO/H1J, ALR/LtJ, NON/LtJ, KK.Cg-AALR/LtJ, NON/LtJ, KK.Cg-Ay/J, B6.HRS(BKS)-Cpefat/+, B6.129P2-Gcktm/Efr, B6.V-Lepob, BKS.Cg-m+/+Leprdb, and C57BL/6J with Diet Induced Obesity.

A. Assessing Effects on Appetite, Including Food Consumption.

The effect of a test compound (e.g., PPAR alpha agonist, OEA-like compound, OEA-like agonist, OEA-like appetite reducing compounds, cannabinoid receptor antagonists, FAAH inhibitor) Or combination of such compounds or combination therapy with such compounds on an appetite for appetizing substance (e.g., sugar, ethanol, a psychoactive substance such as nicotine, narcotics, opiates, CNS stimulants or depressants, anxyiolytic) can be assessed, for instance, by monitoring the consumption of the substance by test subjects (e.g., measuring the amount (e.g., by volume or weight) Consumed or used or not consumed and not used, use of consumption diaries) Or tissue levels (e.g., blood, plasma) Or excretion levels (e.g., urine, feces levels) Of the appetitive substance or its metabolites or by monitoring behaviors seeking the appetitive substance. The effect of the compounds on appetite can also be assessed by subjective means including questionnaires as to appetite or cravings levels by human subjects. The techniques for these assessments are well known to those of ordinary skill in the art. The studies may be acute, subacute, chronic, or subchronic with respect to the duration of administration and or follow-up of the effects of the administration. See also U.S. Pat. No. 6,344,474.

The effect of a candidate compound (e.g., PPAR alpha agonist, OEA-like compounds, OEA-like agonist, OEA-like appetite reducing compounds, cannabinoid receptor antagonists, FAAH inhibitor) Or combination of compounds or combination therapy on the appetite for food or in inducing hypophagia or reduced food intake can be directly assessed, for instance, by monitoring the food consumption of the test subjects (e.g., measuring the amount eaten or not eaten by a subject in terms of food weight or caloric content). The effect on food consumption can be indirectly measured by monitoring body weight. The effect of the compounds on appetite can also be assessed by food consumption diaries, or subjective means including questionnaires as to appetite or food cravings levels by human subjects. The techniques for these assessments are well known to those of ordinary skill in the art. The studies may be acute, subacute, chronic, or subchronic with respect to the duration of administration and or follow-up of the effects of the administration.

B. Assessing Effects on Body Fat Reduction.

Effects on body fat can be identified in vivo using animal bioassay techniques well known to those of ordinary skill in the art. Body fat reduction is typically determined by direct measurements of the change in body fat or by loss of body weight. Body fat and/or body weight of the animals is determined before, during, and after the administration of the candidate compound. Test compounds and appropriate vehicle or caloric controls can be administered by any of a number of routes (e.g., the oral route, a parenteral route) to experimental subjects and the weight of the subjects can be monitored over the course of therapy. The experimental subjects can be humans as well as surrogate test animals (e.g., rats, mice).

Changes in body fat are measured by any means known in the art such as, for example, fat fold measurements with calipers, bioelectrical impedance, hydrostatic weighing, or dual x-ray absorbiometry. Preferably animals demonstrate at least 2%, 5%, 8%, or 10% loss of body fat. Changes in body weight can be measured by any means known in the art such as, for example, on a portable scale, on a digital scale, on a balance scale, on a floor scale, or a table scale. Preferably animals demonstrate at least 2%, 5%, 10%, or 15% loss of body weight. Body weight reduction is measured before administration of the candidate compound and at regular intervals during and after treatment. Preferably, body weight is measured every 5 days, more preferably every 4 days, even more preferably every 3 days, yet more preferably every 2 days, most preferably every day.

For instance, the effect of the candidate compound on total body fat can be determined by taking direct measurements of the rat's body fat using skin fold calipers. Skin on the subjects' backs, abdomen, chest, front and rear legs can be pinched with calipers to obtain measurements before administration of the test compound and at daily or longer intervals (e.g., every 48 hours) during and after administration of candidate compounds. Differences in measurements in one or more of the “pinched” sites reflect the change in the rat's total body fat. The animal may selected from any test species, including but not limited to, mammals, the mouse, a rat, a guinea pig, or a rabbit. The animal may also be an ob/ob mouse, a db/db mouse, or a Zucker rat or other animal model for a weight-associated disease. Clinical studies in humans may also be conducted. In humans, body density measurements or estimates of percent body fat may also be used to assess body fat reduction.

C. Assessing Effects on Lipid Metabolism.

Candidate compounds (e.g., PPARα agonists, OEA-like compounds, OEA-like agonists, OEA-like appetite reducing compounds, cannabinoid receptor antagonists, FAAH inhibitors) and combinations of compound or combination therapies can also be assayed for their effect on fatty acid metabolism. The effect of the candidate compound on fatty acid metabolism can be measured by measurements of fatty acid oxidation in primary cultures of liver cells as taught for instance in U.S. Patent application Ser. No. 10/112,509 filed on Mar. 27, 2002 and assigned to the same assignee as the present application and incorporated by reference.

Changes in fatty acid metabolism can be measured, for instance, by looking at fatty acid oxidation in cells from major fat burning tissues such as, for example, liver (Beynen, et al., Diabetes, 28:828 (1979)), muscle (Chiasson Lab. Anat. of Rat (1980)), heart (Flink, et al., J. Biol. Chem., 267: 9917 (1992)), and adipocytes (Rodbell, J. Biol. Chem., 239: 375 (1964)), Cells may be from primary cultures or from cell lines. Cells may be prepared for primary cultures by any means known in the art including, for example, enzymatic digestion and dissection. Suitable cell lines are known to those in the art. Suitable hepatocyte lines are, for example, Fao, MH1C1, H-4-II-E, H4TG, H4-II-E-C3, McA-RH7777, McA-RH8994, N1-S1 Fudr, N1-S1, ARL-6, Hepa 1-6, Hepa-1c1c7, BpRc1, tao BpRc1, NCTC clone 1469, PLC/PRF/5, Hep 3B2.1-7 [Hep 3B], Hep G2 [HepG2], SK-HEP-1, WCH-17. Suitable skeletal muscle cell lines are, for example, L6, L8, C8, NOR-10, BLO-11, BC3H1, G-7, G-8, C2C12, P19, Sol8, SJRH30 [RMS 13], QM7. Suitable cardiac cell lines are, for example, H9c2(2-1), P19, CCD-32Lu, CCD-32Sk, Girardi, FBHE. Suitable adipocyte lines are, for example, NCTC clone 929 [derivative of Strain L; L-929; L cell], NCTC 2071, L-M, L-M(TK−) [LMTK−; LM(tk−)], A9 (APRT and HPRT negative derivative of Strain L), NCTC clone 2472, NCTC clone 2555, 3T3-L1, J26, J27-neo, J27-B7, MTKP 97-12 pMp97B [TKMp97-12], L-NGC-5HT2, Ltk-11, L-alpha-1b, L-alpha-2A, L-alpha-2C, B82.

The rate of fatty acid oxidation may be measured by 14C-oleate oxidation to ketone bodies (Guzmán and Geelen Biochem. J. 287:487 (1982)) and/or 14C-oleate oxidation to CO₂ (Fruebis, PNAS, 98:2005 (2001); Blazquez, et al., J. Neurochem, 71: 1597 (1998)). Lypolysis may be measured by fatty acid or glycerol release by using appropriate labeled precursors or spectrophotometric assays (Serradeil-Le Gal, FEBS Lett, 475: 150 (2000)). For analysis of 14C-oleate oxidation to ketone bodies, freshly isolated cells or cultured cell lines can be incubated with 14C-oleic acid for an appropriate time, such as, for example, 30, 60, 90, 120, or 180 minutes. The amount of 14C radioactivity in the incubation medium can be measured to determine their rate of oleate oxidation. Oleate oxidation can be expressed as nmol oleate produced in x minutes per g cells. For analysis of lypolysis/glycerol release, freshly isolated cells or cultured cells lines can be washed then incubated for an appropriate time. The amount of glycerol released into the incubation media can provide an index for lypolysis.

III. PPAR Receptor Modulation or Binding Assays.

Methods of characterizing the PPAR receptor binding of compounds are well known to one of ordinary skill in the art. Such methods are readily adaptable for the various subtypes. The methods below exemplify such methods as applied to the PPARα receptor. The results (e.g., affinity measures) Obtained for binding to various PPAR receptor subtypes can be compared to the results obtained for PPARα to determine the specificity of the binding of an agent for PPARα. A preferred measure for comparison is the affinity of the agent for the receptor. Affinity may be measured directely according to the concentration of an agent that gives half-maximal binding or occupancy of the agent to the receptor (e.g., a binding EC₅₀) Or gives a half-maximum inhibition of a competing ligand's binding to the receptor (e.g., IC₅₀). Methods for assessing the relative specificity of a ligand for particular receptors are also well known in the art.

One of ordinary skill in the art would appreciate that a variety of PPARα agonists/PPARα receptor agonists would be useful in the present invention. The ability of a compound (e.g., OEA-like compound, OEA-like appetite reducing compound, or OEA-like agonist) to specifically bind PPARα can be accomplished by any means known in the art, such as, for example, electrophoretic mobility shift assays and competitive binding assays. Preferably PPARα specific binding compounds have at least 5-10 fold, preferably 10-100 fold, more preferably 100-500 fold, most preferably greater than 1000 fold specificity for PPARα compared to other PPAR subtypes. Mammalian PPAR subtypes (e.g., rat, mouse, hamster, rabbit, primate, guinea pig) are preferably used. More preferably, human PPAR subtypes are used.

Electrophoretic Mobility Shift Assays

Electrophoretic mobility shift assays can be used to determine whether test compounds bind to PPARα and affect its electrophoretic mobility. (Forman, et al., PNAS, 94:4312 (1997) and Kliewer, et al., PNAS, 91:7355 (1994)). Electrophoretic mobility shift assays involve incubating a PPAR-RXR with a test compound in the presence of a labeled nucleotide sequence. Labels are known to those of skill in the art and include, for example, isotopes such as, ³H, ¹⁴C, ³⁵S, and ³²P, and non-radioactive labels such as fluorescent labels or chemiluminescent labels. Fluorescent molecules which can be used to label nucleic acid molecules include, for example, fluorescein isothiocyanate and pentafluorophenyl esters. Fluorescent labels and chemical methods of DNA and RNA fluorescent labeling have been reviewed recently (Proudnikov, et al., Nucleic Acids Res., 24:4535-42 (1996)).

Chemiluminescent labels and chemiluminescent methods of labeling DNA and RNA have been reviewed recently (Rihn, et al., J. Biochem. Biophys. Methods, 30:91-102 (1995)). Use of non-radioactive labeled probes directly for studying protein-polynucleotide interactions with EMSA has been described. (U.S. Pat. No. 5,900,358). The mixtures can be separated, run on a separate lane of a gel, and autoradiographed. For example, if a test compound does not result in a change in the bands seen in the control lane then the test compound is not a candidate PPARα specific binding compound. On the other hand, if a change in intensity in at least one of the bands is seen, then the compound is a candidate PPARα specific binding compound. (U.S. Pat. No. 6,265,160). The incubation mixture is then electrophoretically separated and the resulting gel exposed to X-ray film. The resulting autoradiograph may have one or more bands representing slowly migrating DNA-protein complexes. This control lane can indicate the mobility of the complex between the DNA probe and the particular PPAR.

Monoclonal antibodies specific for PPAR subtypes can be used to identify PPARα specific binding compounds in modified electrophoretic mobility shift assays. Purified PPARβ, PPARα or PPARγ can be incubated with an appropriate amount of a test compound in the presence of RXR. For these assays, the test compound need not be labeled. PPAR subtype specific monoclonal antibodies can be incubated with the PPAR-RXR-test compound mixture. For instance, test compounds that bind PPAR induce supershifting of the PPAR-RXR complex on a gel (Forman, et al. (1997), PNAS 94:4312) which can be detected by anti-PPAR monoclonal antibodies using a Western blot (immunoblot).

Generation of monoclonal antibodies has been previously described and can be accomplished by any means known in the art. (Buhring et al. in Hybridoma 1991, Vol. 10, No. 1, pp. 77-78). For example, an animal such as a guinea pig or rat, preferably a mouse is immunized with a purified PPAR subtype, the antibody-producing cells, preferably splenic lymphocytes, are collected and fused to a stable, immortalized cell line, preferably a myeloma cell line, to produce hybridoma cells which are then isolated and cloned. (U.S. Pat. No. 6,156,882).

Western blots generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind PPAR subtypes. These antibodies may be directly labeled or alternatively may be subsequently detected using labeled antibodies (e.g., labeled sheep anti-mouse antibodies) that specifically bind to the anti-PPAR antibodies.

The particular label or detectable group used in the assay is not a critical aspect of the invention, as long as it does not significantly interfere with the specific binding of the PPAR subtype specific ligand used in the assay. The detectable group can be any material having a detectable physical or chemical property. Thus, a label is any composition detectable by spectroscopic, photochemical, biochemical, electrical, optical or chemical means. A wide variety of labels may be used, with the choice of label depending on sensitivity required, ease of conjugation with the compound, stability requirements, available instrumentation, and disposal provisions. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), and colorimetric labels such as colloidal gold or colored glass or plastic beads (e.g., polystyrene, polypropylene, latex, etc.).

The molecules can be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Other assay formats include liposome immunoassays (LIA), which use liposomes designed to bind specific molecules (e.g., antibodies) and release encapsulated reagents or markers. The released chemicals can be then detected according to standard techniques (see, Monroe, et al., Amer. Clin. Prod. Rev., 5:34-41 (1986)).

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Competitive Binding Assays

In addition to electrophoretic mobility shift assays, competitive binding assays can be used to identify PPARα specific binding compounds. In competitive assays, the binding of test compounds to PPARα can be determined by measuring the amount of OEA that they displaced (competed away) from PPARα. Purified PPARβ, PPARα, and PPARγ receptors can be incubated with varying amounts of a test compound in the presence of labeled ligands specific for each PPAR subtype. For example, GW 2433 and L-783483 can be used in conjunction with PPARβ; GW 2331 or OEA can be used in conjunction with PPARα; and rosiglitazone, AD-5075, and SB-236636 can be used in conjunction with PPARγ. Specificity of the test compound for each PPAR subtype can be determined by detection of the amount of labeled ligand that remains bound to each PPAR after incubation with the test compound. Labels are discussed above.

High Throughput Screening of Candidate Compounds that Specifically Bind PPARα

In conjunction with the methods described above, identification of OEA-like compounds and OEA-like modulators can be accomplished via high throughput screening. Conventionally, new chemical entities with useful properties can be generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

High throughput screening methods involve providing a library containing a large number of potential PPARα specific binding compounds (candidate compounds). Such “combinatorial chemical libraries” can be then screened in one or more assays, as described herein, to identify those library members (particular chemical species or subclasses) that display a desired characteristic activity. The compounds thus identified can serve as conventional “lead compounds” or can themselves be used as potential or actual therapeutics.

a. Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library can be formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9):1233).

Preparation and screening of combinatorial chemical libraries are well known to those of skill in the art. Such combinatorial chemical libraries include, but are not limited to, benzodiazepines (U.S. Pat. No. 5,288,514), diversomers such as hydantoins, benzodiazepines and dipeptides (Hobbs et al. (1993) PNAS. USA 90: 6909), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658), and small organic molecule libraries (see, e.g., benzodiazepines, Baum (1993) C&EN, Jan 18, page 33, thiazolidinones and metathiazanones U.S. Pat. No. 5,549,974, pyrrolidines U.S. Pat. Nos. 5,525,735 and 5,519,134, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky.; Symphony, Rainin, Woburn, Mass.; 433A, Applied Biosystems, Foster City, Calif., 9050; Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J.; Asinex, Moscow, Ru; Tripos, Inc., St. Louis, Mo.; ChemStar, Ltd., Moscow, RU; 3D Pharmaceuticals, Exton, Pa.; Martek Biosciences, Columbia, Md.; etc.).

b. High Throughput Assays of Chemical Libraries

Many of the in vitro assays for compounds described herein are amenable to high throughput screening. Preferred assays thus detect activation of transcription (i.e., activation of mRNA production) by the test compound(s), activation of protein expression by the test compound(s), or binding to the gene product (e.g., expressed protein) by the test compound(s).

High throughput assays for the presence, absence, or quantification of particular protein products or binding assays are well known to those of skill in the art. Thus, for example, U.S. Pat. No. 5,559,410 discloses high throughput screening methods for proteins, and U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

IV. Measuring Activation of PPAR Subtypes, Including PPARα.

One of ordinary skill in the art would know how to test a compound for its PPAR modulatory and activation activity for any of the PPAR receptor subtypes, including PPARα Such methods can be used to identify a compound as an agonist of any of the PPAR receptors. See for instance, Willson et al., J. Med. Chem. 43(4): 527-549 (2000) and Kliewer et al. Proc. Natl. Acad of Sci, USA 91:7355-7359 (1994). Comparison of the concentration dependence of a compound's ability to activate the PPARα receptor to that of other PPAR receptor subtypes can be used to identify a selective or specific PPARα receptor agonist. The following methods set forth for PPARα exemplify such methods in general and can be readily adapted to the other PPAR receptor subtypes by one of ordinary skill.

The ability of a candidate PPAR agonist, OEA-like compound or OEA-like modulator to activate PPARα can be measured using any means known in the art. PPARα activators act by inducing PPARα-RXR heterodimer formation. The PPARα-RXR heterodimer then binds to DNA sequences containing AGGTCAnAGGTCA and activates PPAR target genes. Preferably PPARα activators activate PPARα by at least 5-10 fold, more preferably 10-100 fold, more preferably 100-500 fold, more preferably 500-100 fold, most preferably greater than 1000 fold above base level. PPARα can be transfected into cells. The transfected cells can be then exposed to candidate compounds. Any means known in the art can be used to determine whether PPARα is activated by the candidate compound, such as for example, by measuring levels of reporter gene expression and cell proliferation.

Transfection of PPAR into Cells

Any of the well-known procedures for introducing foreign nucleotide sequences into host cells may be used to transfect PPARα into cells such as, for example, calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook, et al., supra). Methods of transfection have also been described in U.S. Pat. Nos. 5,616,745; 5,792,652; 5,965,404, and 6,051,429 and in Current Protocols in Molecular Biology, Ausubel, et al., ed. (2001). It is only necessary that the particular genetic engineering procedure used be capable of successfully introducing at least one gene into the host cell capable of expressing PPARα After the expression vector is introduced into the cells, the transfected cells can be cultured under conditions favoring expression of PPARα.

Detection of Reporter Gene Expression

Expression of reporter genes in response to compounds identified as binders of PPARα may also be used to measure PPARα activation. PPARα may be co-transfected with reporter genes known in the art such as, for example, luciferase, β-galactosidase, alkaline phosphatase, fluorescent green protein, or chloramphenicol acetyltransferase. The transfected cells can be exposed to appropriate concentrations of candidate compounds with OEA as a positive control. Reporter gene expression will be induced by compounds that bind and activate PPARα. Thus, compounds that induce reporter gene expression can be identified as activators of PPARα (Forman, et al. (1997) PNAS 94:4312). Preferably the compounds induce reporter gene expression at levels at least 5-10 fold, more preferably 10-100 fold, more preferably 100-500 fold, more preferably 500-1000 fold, most preferably greater than 1000 fold greater than the negative control.

Proliferation of PPARα Transfected Cells

PPARα activation may also be measured by proliferation of cells transfected with PPARα Cell proliferation can be induced by compounds that bind and activate PPARα, such as, for example, OEA. Thus, PPARα transfected cells can be exposed to appropriate concentrations of candidate compounds with OEA as a positive control. Compounds that induce cells to proliferate can thereby be identified as activators of PPARα. Cell proliferation can be measured, for example, by incorporation of 5′-bromo-2′deoxyuridine or 3H-thymidine as described in Jehl-Pietri, et al., (2000) Biochem J. 350:93 and Zoschke and Messner (1984) Clin. Immunol. Immunopath. 32:29, respectively. Preferably the compounds induce cell proliferation at levels at least 5-10 fold, more preferably 10-100 fold, more preferably 100-500 fold, more preferably 500-1000 fold, most preferably greater than 1000 fold greater than the negative control.

V. Cannabinoid Receptor Antagonism Bioassays.

One of ordinary skill in the art would appreciate that a variety of CB1 receptor antagonists would be useful in the present invention. Preferably, the antagonists have a greater selectivity for the CB1 cannabinoid receptor than the CB2 cannabinoid receptor. In some embodiments, for instance, the antagonist has at least a four-fold lower IC₅₀ or Ki for a CB1 cannabinoid receptor than the CB2 cannabinoid receptor. In other embodiments, the antagonist has at least a ten-fold-fold lower IC₅₀, or Ki, for a CB1 cannabinoid receptor than the CB2 cannabinoid receptor. In still other embodiments, the antagonist has at least a 20-fold-fold lower IC₅₀, or Ki, for a CB1 cannabinoid receptor than the CB2 cannabinoid receptor according to any of the physiologically relevant methods for studying such binding, and, more particularly, such assays as described herein or incorporated by reference.

A first group of suitable cannabinoid CB1 receptor antagonists are pyrazole derivatives. Patent applications EP-A-576 357 and EP-A-658 546 describe exemplary pyrazole derivatives which have an affinity for the cannabinoid receptors. More particularly, patent application EP-A-656 354 discloses exemplary pyrazole derivatives and claims N-piperidino-5-(4-chlorophenyl)-1-(2,4-dichlorophenyl)-4-methylpyrazole-3-carboxamide, or SR 141716, and its pharmaceutically acceptable salts, which have a very good affinity for the central cannabinoid receptors. Additonal exemplary CB1 receptor antagonists are disclosed in U.S. Pat. No. 5,596,106 which discloses both arylbenzo[b]thiophene and benzo[b]furan compounds to block or inhibit cannabinoid receptors in mammals. Preferably, such a cannabinoid antagonist is selective for the CB1 receptor and has an IC₅₀ for the CB1 receptor which is one-fourth or less than that of the CB2 receptor or, more preferably, is one-tenth or less than the IC₅₀ for the CB2 receptor, or even more preferably, an IC₅₀ with respect to the CB1 receptor which is one-hundredth that for the CB2 receptor. Each of the above references is incorporated by reference in its entirety.

In some embodiments, the CB1 cannabinoid receptor antagonist poorly penetrates the blood brain barrier. In other embodiments, the CB1 cannabinoid receptor antagonist bears a net positive charge at physiological pH. In some embodiments, the CB1 cannabinoid receptor does not significantly act upon central CB1 cannabinoid receptors upon systemic or non-central administration.

In one embodiment, the cannabinoid CB1 receptor antagonist is a 4,5,dihydro-1H-pyrazole derivative having CB1-antagonistic activity as taught in U.S. Patent Application No. 2001/0053788A1 and particularly disclosed by formula (I) therein. U.S. Patent Application No. 2001/0053788A1 published on Dec. 20, 2001 and is incorporated by reference in its entirety.

Also useful are the cannabinoid CB1 receptor antagonist compounds of the formula 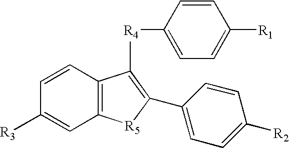
wherein the substituents R₁, R₂, R₃, R₄, and R₅ are defined as recited in U.S. Pat. No. 5,596,106 which is incorporated by reference in its entirety. Related reference U.S. Pat. No. 5,747,524 is also incorporated by reference in its entirety. This reference discloses additional exemplary aryl-benzo[b]thiophene and arylbenzo[b]furan derivatives for use according to the invention.

The cannabinoid antagonists of the following formula are also particularly useful according to the invention: 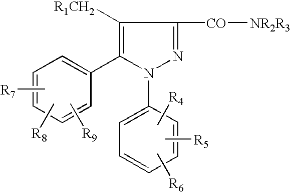
wherein R₁ is hydrogen, a fluorine, a hydroxyl, a (C₁-C₅)alkoxy, a (C₁-C₅)alkylthio, a hydroxy(C₁-C₅)alkoxy, a group —NR₁₀R₁₁, a cyano, a (C₁-C₅)alkylsulfonyl or a (C₁-C₅)alkylsulfinyl;
R₂ and R₃ are a (C₁-C₄)alkyl or, together with the nitrogen atom to which they are bonded, form a saturated or unsaturated 5- to 10-membered heterocyclic radical which is unsubstituted or monosubstituted or polysubstituted by a (C₁-C₃)alkyl or by a (C₁-C₃)alkoxy;
R₄, R₅, R₆, R₇, R₈ and R₉ are each independently hydrogen, a halogen or a trifluoromethyl, and if R₁ is a fluorine, R₄, R₅, R₆, R₇, R₈ and/or R₉ can also be a fluoromethyl, with the proviso that at least one of the substituents R₄ or R₇ is other than hydrogen; and
R₁₀ and R₁₁ are each independently hydrogen or a (C₁-C₅)alkyl, or R₁₀ and R₁₁, together with the nitrogen atom to which they are bonded, form a heterocyclic radical selected from pyrrolidin-1-yl, piperidin-1-yl, morpholin-4-yl and piperazin-1-yl, which is unsubstituted or substituted by a (C₁-C₄)alkyl,
and their salts and their solvates.

More particularly, the present invention relates to the use of N-piperidino-5-(4-chlorophenyl)-1-(2,4-dichlorophenyl)-4-methylpyrazole-3-carboxamide, its pharmaceutically acceptable salts and their solvates for the preparation of drugs useful in the treatment of appetency disorders. This particularly preferred cannabinoid antagonist is SR 141616 and is of the formula:

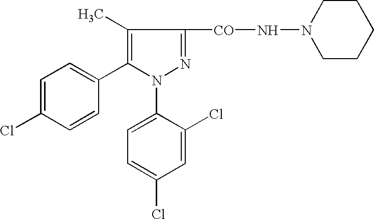

Another group of exemplary cannabinoid CB1 receptor antagonists for use according to the invention are pyrazole derivatives according to Formula (I) Of U.S. Pat. No. 6,028,084 which is incorporated by reference in its entirety.

U.S. Pat. No. 6,017,919 discloses another group of suitable cannabinoid receptor antagonists for use according to the invention. These antagonists are of the following general formula: 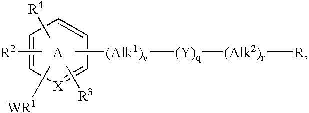
wherein the substituents are as defined in U.S. Pat. No. 6,017,919 which is incorporated herein by reference in its entirety.
VI. Cannabinoid Receptor Activity Screening.

A variety of means may be used to screen cannabinoid CB1 receptor activity in order to identify the compounds according to the invention. A variety of such methods are taught in U.S. Pat. No. 5,747,524 and U.S. Pat. No. 6,017,919.

A. Ligand Binding Assays.

Ligand binding assays are well known to one of ordinary skill in the art. For instance, see, U.S. Patent Application No. US 2001/0053788 published on Dec. 20, 2001, U.S. Pat. No. 5,747,524, and U.S. Pat. No. 5,596,106 and (see, Felder, et al., Proc. Natl. Acad. Su., 90:7656-7660 (1993)) each of which is incorporated herein by reference. The affinity of an agent for cannabinoid CB1 receptors can be determined using membrane preparations of Chinese hamster ovary (CHO) Cells in which the human cannabis CB1 receptor is stably transfected in conjunction with [³H]CP-55,940 as radioligand. After incubation of a freshly prepared cell membrane preparation with the [³H]-ligand, with or without addition of compounds of the invention, separation of bound and free ligand can be performed by filtration over glassfiber filters. Radioactivity on the filter was measured by liquid scintillation counting.

The cannabinoid CB1 antagonistic activity of a candidate compound for use according to the invention can also be determined by functional studies using CHO cells in which human cannabinoid CB1 receptors are stably expressed. Adenylyl cyclase can be stimulated using forskolin and measured by quantifying the amount of accumulated cyclic AMP. Concomitant activation of CB1 receptors by CB1 receptor agonists (e.g., CP-55,940 or (R)-WIN-55,212-2) Can attenuate the forskolin-induced accumulation of cAMP in a concentration-dependent manner. This CB1 receptor-mediated response can be antagonized by CB1 receptor antagonists. See, U.S. Patent Application No. US 2001/0053788 published on Dec. 20, 2001.

Samples rich in cannabinoid CB1 receptors and CB2 receptors, rat cerebellar membrane fraction and spleen cells can be respectively used (male SD rats, 7-9 weeks old). A sample (cerebellar membrane fraction: 50 μ.g/ml or spleen cells: 1(×10⁷ cells/ml), labeled ligand ([3H]Win55212-2, 2 nM) and unlabeled Win55212-2 or a test compound can be plated in round bottom 24 well plates, and incubated at 30° C. for 90 min in the case of cerebellar membrane fraction, and at 4° C. for 360 min in the case of spleen cells. As the assay buffer, 50 mM Tris solution containing 0.2% BSA can be used for cerebellar membrane fraction, and 50 mM Tris-HBSS containing 0.2% BSA can be used for spleen cells. After incubation, the samples are filtrated through a filter (Packard, Unifilter 24 GF/B) and dried. A scintillation solution (Packard, Microsint-20) Can be added, and the radioactivity of the samples determined (Packard, Top count A9912V). The non-specific binding can be determined by adding an excess Win55212-2 (1 μM), and calculating specific binding by subtracting non-specific binding from the total binding obtained by adding the labeled ligand alone. The test compounds can be dissolved in DMSO to the final concentration of DMSO of 0.1%. IC₅₀ can be determined from the proportion of the specifically-bound test compounds, and the Kᵢ value of the test compounds can be calculated from IC₅₀ and K_{d} value of [3H]WIN55212-2. See, U.S. Pat. No. 6,017,919.

In one embodiment, the IC₅₀ for cannabinoid receptor binding is determined according to the method of Devane, et al., Science, 258: 1946-1949 (1992) and Devane, et al., J. Med. Chem., 35:2065 (1992). In this method, the ability of a compound to competively inhibit the binding of a radiolabeled probe (e.g., ³H-HU-2430) is determined.

In other embodiments, the IC₅₀ of an inventive compound for the CB1 receptor is determined according to any one of the above ligand binding assay methods. In another embodiment, the IC₅₀ is according to any assay method which studies binding at physiological pH or physiologically relevant conditions. In another embodiment, the IC₅₀ is determined according to any assay method which studies binding at physiological pH and ionic strength. Preferred assay incubation temperatures range from 20° C.-37° C. Temperatures may be lower or higher. For instance, incubation temperatures of just a few degree or 0° C. may be useful in preventing or slowing the degradation of enzymatically unstable ligands. Inhibitors of FAAH may also be added to protect antagonists from degradation.

B. Effect on N-Type Calcium Channel Currents.

Cannabinoid antagonist activity can also be assessed by studying inhibition of the signal transduction pathway of the CB1 receptor, when activated by its endogenous ligand, anandamide, but in addition, effect other nerve cell organelles under control of the CB1 signaling pathway in vitro. Specifically, the antagonists can open the N-type calcium channels, which are closed by either anandamide or the cannabinoids (see, Mackie, K. and Hille, B., Proc. Natl. Acad. Sci., 89:3825-3829 (1992)). See, U.S. Pat. No. 5,596,106 which is incorporated herein by reference which teaches how to identify CB1 antagonists on nerve cells by measuring current flow using a whole-cell voltage-clamp technique. A cannabinoid agonist (e.g., amandamide or WIN 55,212 will inhibit the N-type calcium channel via the CB1 receptor, thus decreasing the current to the voltage clamp of −65 pA. The addition of an CB1 receptor antagonist will oppose the action of the agonist.

A variety of means may be used to screen cannabinoid CB2 receptor activity in order to identify the compounds according to the invention.

C. Cannabinoid CB2 Receptor Binding Assay.

Methods of studying CB2 receptor binding are well known to one of ordinary skill in the art. For instance, binding to the human cannabinoid CB2 receptor can be assessed using the procedure of Showalter, et al., J. Pharmacol Exp Ther., 278(3):989-99 (1996)), with minor modifications as taught for instance in U.S. Patent Application No. 20020026050, published Feb. 28, 2002. Each of which is incorporated herein by reference.

In other embodiments, the IC₅₀ of an inventive compound for the CB2 receptor is determined according to any one of the above CB2 receptor ligand binding assay methods. In another embodiment, the IC₅₀ is according to any assay method which studies binding at physiological pH or physiologically relevant conditions. In another embodiment, the IC₅₀ is determined according to any assay method which studies binding at physiological pH and ionic strength. Preferred assay incubation temperatures range from 20° C.-37° C. Temperatures may be lower or higher. For instance, incubation temperatures of just a few degree or 0° C. may be useful in preventing or slowing the degradation of enzymatically unstable ligands. Inhibitors of FAAH may also be added to protect antagonists from degradation.

Methods for identification and assaying FAAH inhibitors are set forth in Example VI.

D. Determining the Combination Therapy Dosages.

Preferred dosages of the cannabinoid receptor antagonist and PPARα receptor agonist or OEA-like appetite reducing compound or FAAH inhibitor to be used in a combination therapy can be determined experimentally by first conducting separate dose response studies for the cannabinoid receptor antagonist and PPARα receptor agonist, OEA-like appetite reducing compound, or FAAH inhibitor to be used. Methods of performing such dose response studies in a test species or the species of the intended subject (e.g., a human) are well known to one of ordinary skill in the art. The endpoint of the study is preferably selected according to the effect or endpoint of interest (e.g., appetite reduction, weight loss, body fat reduction, changes in lipid metabolism, changed food seeking behavior) Or the dose response of the underlying mechanism of action (e.g., receptor activation or antagonism). Alternatively, the established dose response relationships may be used if an agent is already well-characterized as to dose response. Preferred bioassay methods include those described above and those presented in the Examples.

The dosages suitable for the combination therapy are then selected so as to provide room for the synergism to operate. The preferred dosage for each agent is identified from the dose response curve and corresponds to one providing a submaximal effect when given alone. A submaximal dosage would leave the most room for synergism beween the cannabinoid receptor antagonist and PPARα receptor agonist to occur. Preferably, therefore, the dosage for at least one of each such agent is below the dosage providing a 50% maximum effect for that agent when given alone. More preferably, both the the cannabinoid receptor antagonist and PPARα receptor agonist are each administered in a dosage corresponding to the dosage providing less than a 50% maximum effect for each such agent when administered alone. More preferably, the dosage for at least one (or both) Of each such agent is below the dosage providing a 25% or 10% maximum effect for each of the cannabinoid receptor antagonist and PPARα receptor agonist when given alone. More preferably, at least one or both of the doses or amounts of the cannabinoid antagonist to be administered and the doses or amounts of the PPARα agonist to be admininistered are subthreshold doses. Confirmation of the synergism can be confirmed by comparing the effect of the combination therapy to the effects of the individual compounds alone. Synergism is observed when the combined effects are greater than the effect expected when the effects of the same amounts of the individual compounds administered alone are added.

VII. Methods of Use, Pharmaceutical Compositions, and their Administration.

A. Methods of Use.

Compositions comprising either or both of the CB1 cannabinoid receptor antagonist and the PPARα agonist (e.g., OEA-like agonist, OEA-like compound) Or OEA-like appetite reducing compound or FAAH inhibitor may be administered in a combination therapy to control or reduce appetite for food or to treat appetency disorders in a mammal, preferably a human. The compositions may be administered to reduce body fat and or body weight in mammals, including dogs, cats, and especially humans. Alternatively, the PPARα agonist (e.g., OEA-like agonist, OEA-like compound) Or OEA-like appetite reducing compound or FAAH inhibitor and cannabinoid CB1 receptor antagonists may be administered separately to reduce an appetite for an appetizing substance or to treat appetency disorders or to reduce body fat and or body weight in mammals, including dogs, cats, and especially humans. The weight loss may be for aesthetic or for therapeutic purposes. The compounds may also be used to reduce the appetite food or induce hypophagia. The inventive methods and compositions and combination therapy may be used to treat appetency disorders and reduce the desire for psychoactive substances especially in the treatment of addictive disorders related to addictive substances (e.g, psychoactive substances such as narcotics, nicotine or tobacco products, CNS stimulants, and CNS depressants).

The combination therapy methods and compositions of the present invention act selectively, for instance, on consumption behavior disorders pertaining to appetizing substances. Thus the administration of the inventive compositions and such compounds makes it possible to regulate the desire to consume non-essential food items such as excess sugars, excess carbohydrates, fats, alcohol or drugs.

The CB1 receptor antagonist and PPARα agonist (e.g., OEA-like agonist, OEA-like compound) Or OEA-like appetite reducing compound or FAAH inhibitors and compositions and combination therapies of the invention are particularly useful to prevent weight gain or body fat increases in individuals within a normal weight range. Such compounds and compositions may be used in otherwise healthy individuals who are not otherwise in need of any pharmaceutical intervention for diseases related to diabetes or hyperlipidemia or cancer. In some embodiments, the individuals to be treated are free of diseases related to disturbances in sugar or lipid levels or metabolism or free of risk factors for cardiovascular and cerebrovascular disease. The individuals, for instance, can be non-diabetic and have blood sugar levels in the normal range. The individuals can also, for example, have blood lipids (e.g., cholesterol) Or triglyceride levels in the normal range. The individuals may be free of atherosclerosis. In some embodiments, the individuals can be free of other conditions such as cancer or other tumors, disorders involving insulin resistance, Syndrome X, and pancreatitis.

In other embodiments, the subjects are overweight or obese persons in need of body fat and/or body weight reduction. In these embodiments, the methods, compounds, and compositions of the invention can be administered to promote weight loss and also to prevent weight gain once a body weight within the normal range for a person of that sex and age and height has been achieved. The compounds and compositions may be used in otherwise healthy individuals who are not in need of any pharmaceutical treatment of a disorder related to diabetes, hyperlipidemia, or cancer. The individuals may also otherwise free of risk factors for cardiovascular and cerebrovascular diseases. In some embodiments, the individuals to be treated are free of diseases related to sugar (e.g., glucose) Or lipid metabolism. The individuals may be non-diabetic and have blood sugar levels in the normal range. The individuals may also have blood lipids (e.g., cholesterol, HDL, LDL, total cholesterol) Or triglyceride levels in the normal range. The individuals may not need to be in treatment for atherosclerosis.

The CB1 receptor antagonist and PPARα agonist (e.g., OEA-like agonist, OEA-like compound) Or OEA-like appetite reducing compound or FAAH inhibitor and compositions of the invention may also be administered to suppress food appetite in mammals, including cats, dogs, and humans. In some embodiments, the compounds may be used in otherwise healthy individuals who are not in need of pharmaceutical interventions for any disease. In some embodiments, the individuals do not need preventive or ameliorative therapy for diseases, including cancer, diabetes, or hyperlipidemia. In some embodiments, the individuals to be treated are free of diseases related to abnormal sugar or lipid levels. In other embodiments the individuals may be free of risk factors for cardiovascular or cerebrovascular disease. The individuals may be non-diabetic and have blood sugar levels in the normal range. The individuals may also have blood lipids (e.g., cholesterol) Or triglyceride levels in the normal range. The individuals may be free of atherosclerosis.

The CB1 receptor antagonist and the PPARα agonist (e.g., OEA-like agonist, OEA-like compound) Or OEA-like appetite reducing compound or FAAH inhibitor and compositions of the invention may also be administered in combination therapy to modulate fat metabolism (e.g., increase fat catabolism) in mammals, including cats, dogs, and humans. In some embodiments, the CB1 receptor antagonists and the OEA-like agonists, OEA-like compounds or OEA-like appetite reducing compounds may be used to reduce appetite in otherwise healthy individuals. In some embodiments, the individuals to be treated are free of diseases related to sugar or lipid metabolism (e.g., diabetes, hypercholesterolemia, low HDL levels or high LDL levels). The individuals may be non-diabetic and have blood sugar levels in the normal range. The individuals may also have blood lipids (e.g., cholesterol) Or triglyceride levels in the normal range. The individuals may be free of atherosclerosis.

In some embodiments, combination therapy may be for a period predetermined by the degree or amount of weight loss has been accomplished or when the individual achieves a BMI within the normal range. Treatment with the compounds and compositions of the invention may be reduced once a predetermined degree or amount of weight loss has been accomplished or when the individual achieves a BMI within the normal range.

The CB1 receptor antagonist and PPARα agonist (e.g., OEA-like agonist, OEA-like compound) Or OEA-like appetite reducing compound and compositions may be administered in a combination therapy solely for the purposes of reducing body fat or reducing appetite in individuals not otherwise needing such compositions according to the invention.

The CB1 receptor antagonist and PPARα agonist (e.g., OEA-like agonist, OEA-like compound) Or OEA-like appetite reducing compound or FAAH inhibitor and compositions may administered alone or in combination therapy to treat appetency disorders involving appetizing substances such foods, sugars, alcohols, nicotine, and psychoactive drugs such as CNS stimulants and depressants.

The compounds and compositions of the invention may be used to treat appentency disorders in individuals otherwise not in need of an appetite suppressing fatty acid alkanolamide or homologue or analog.

Marijuana use is associated with loss of sensory perception, cognition, and mood changes such as lethargy and depression. An endogenous controlling factor exacerbating such events would also be an inappropriately high or unregulated control of anandamide-CB1 interaction. A combination therapy of cannabinoid antagonists and PPARα agonist (e.g., OEA-like agonist, OEA-like compound) Or OEA-like appetite reducing compound or FAAH inhibitors would also be useful in conditions where patients exhibit these symptoms.

In each of these aspects, the compositions may be administered by a variety of routes, including, but not limited to, the oral, rectal, topical, parenteral (including subcutaneous, intramuscular, and intravenous), pulmonary (nasal or buccal inhalation), or nasal administration, although the most suitable route in any given case will depend in part on the nature and severity of the conditions being treated and on the nature of the active ingredient. An exemplary route of administration is the oral route.

When administered in combination therapy, both a CB1 receptor antagonist and PPARα agonist (e.g., OEA-like agonist, OEA-like compound) Or OEA-like appetite reducing compound or FAAH inhibitor or compositions thereof are administered to a subject. The administration may be at the same or at different times as long as the antagonist and PPARα agonist (e.g., OEA-like agonist, OEA-like compound) Or OEA-like appetite reducing compound or FAAH inhibitor are present in the body at the same time. In one embodiment of the combination therapy, at least one or both of the CB1 receptor antagonist and the the OEA-like agonist, OEA-like compound) Or OEA-like appetite reducing compound (e.g., appetite supressing fatty acid alkanolamide compound, homologue, or analog) Or FAAH inhibitor is administered in a subthreshold amount. In one embodiment, the administered amount of such compounds may be an effective dose (ED) as judged by a benchmark effect for about or fewer than 1%, 5%, 10%, 25%, or 50% of a recipient population (e.g., recipient population ED₁, ED₅, ED₁₀, ED₂₅, ED₅₀) as judged by the dose response curve for reduction in an appetitive behavior (e.g., consumption of a food or other appetizing substance) in the intended subject population (e.g., humans, primates, mammals, dogs, cats, rats, mice). In some embodiments, the benchmark is a 2%, 5, %, 10%, 50% or greater reduction in an appetitive behavior (e.g., the consumption of the food or appetizing substance) as compared to a control. In other embodiments, these amounts by themselves would have an insignificant or small effect on appetitive behavior (e.g., affecting food consumption or the consumption of an appetitive substance) by less than 1%, 2%, 5%, 10% as compared to a control group. In other embodiments, the amounts by themselves would reduce food consumption or consumption of an appetizing substance by about less than 5%, 10%, 25%, or 50% (biological effect ED₅, ED₁₀, ED₂₅, ED₅₀) Of the maximum effect that can be achieved with higher doses of the same compound under similar experimental or clinical conditions. Such dose response characterizations are well known to one of ordinary skill in the art. In other embodiments of the combination therapy, the antagonist is given in an amount which results in a peak average plasma concentration which is less than one-half, one-third, one-tenth, or one-twentieth the IC₅₀ for the CB1 cannabinoid receptor binding in vitro. Methods of measuring the plasmal level of such drugs and their IC₅₀ in vitro are well known to one of ordinary skill in the art. In some embodiments, the ED values are determined with respect to the particular species (e.g., human, mouse, rat, dog, cat) Of the individual to be treated. In other embodiments, the ED values are determined with respect to the classification to which species belongs (e.g., primate, mammal, rodent).

In some embodiments, a FAAH inhibitor is used in place of or in addition to the PPARα agonist (e.g., OEA-like agonist, OEA-like compound) Or OEA-like appetite reducing compound. Such inhibitors can increase the endogenous level of OEA so as to synergize with an administered CB1-cannabinoid receptor antagonist. In some embodiments, the FAAH inhibitor is administered in addition to the OEA-like compound to increase the ability of the OEA-like compound to synergize with the CB-1 cannabinoid receptor antagonist.

B. Pharmaceutical Compositions.

In another aspect, the present invention provides pharmaceutical compositions which comprise a CB1 cannabinoid receptor antagonist and an PPARα agonist (e.g., OEA-like agonist, OEA-like compound) Or OEA-like appetite reducing compound or FAAH inhibitor as the active ingredients, and may also contain a pharmaceutically acceptable carrier and optionally other therapeutic ingredients.

In another aspect, the present invention provides, a pharmaceutical composition in unit dosage format which comprises a cannabinoid antagonist in an amount which by itself would not be expected to significantly affect appetite or food intake upon administration. In some embodiments, these amounts by themselves are subthreshold amounts. In other embodiments, these amounts by themselves are effective as judged by a benchmark effect for about or fewer than 1%, 5%, 10%, 25%, or 50% of a recipient population (e.g., recipient population ED₁, ED₅, ED₁₀, ED₂₅, ED₅₀) as judged by the dose response curve for reduction in an appetitive behavior (e.g., consumption of a food or other appetizing substance). In some embodiments, the benchmark is a 2%, 5, %, 10%, 50% or greater reduction in an appetitive behavior (e.g., the consumption of the food or appetizing substance) as compared to a control. In some embodiments, the recipient population is a human, a mammal, a mouse, or a rat population. In other embodiments, these amounts by themselves would have an insignificant or small effect on appetitive behavior (e.g., affecting food consumption or the consumption of an appetitive substance) by less than 1%, 2%, 5%, 10% as compared to a control group of the human, a mammal, a mouse, or a rat population. In other embodiments, the amounts by themselves would reduce food consumption or consumption of an appetizing substance by about less than 5%, 10%, 25%, or 50% (biological effect ED₅, ED₁₀, ED₂₅, ED₅₀) Of the maximum effect that can be achieved with higher doses of the same compound under similar experimental or clinical conditions. Such dose response characterizations are well known to one of ordinary skill in the art. In a further embodiment, such CB1 antagonist compositions further comprise an OEA-like appetite reducing compound (e.g., OEA or rimonabant). The amount or dosage of the OEA compound, in some embodiments, is as described herein for the OEA compositions of the invention which lack a CB1 antagonist.

In another aspect, the present invention provides, a pharmaceutical composition comprising a unit dosage of the PPARα agonist (e.g., OEA-like agonist, OEA-like compound) Or OEA-like appetite reducing compound or FAAH inhibitor in an amount which by itself would not be expected to significantly affect appetite or food intake upon administration. In some embodiments, these amounts are subthreshold amounts. In other embodiments, these amounts by themselves are effective with respect to some benchmark effect for fewer than 1%, 5%, 10%, 25%, or 50% of a recipient population as described above (e.g., recipient population ED₁, ED₅, ED₁₀, ED₂₅, ED₅₀) as judged by the dose response curve for reduction in an appetite (e.g., appetite for food or other appetizing substance) with respect to a benchmark effect. In some embodiments, the benchmark is a 2%, 5, %, 10%, 50% or greater reduction in the consumption of the food or appetizing substance as compared to a control. In other embodiments, these amounts by themselves would have an insignificant or small effect on appetite, affecting food consumption by less than 1%, 2%, 5%, 10% as compared to a control group as described above. In other embodiments, the amount by themselves would reduce appetite by about less than 5%, 10%, 25%, or 50% (biological effect ED₅, ED₁₀, ED₂₅, ED₅₀) Of the maximum effect that can be achieved with higher doses of the same compound under similar experimental or clinical conditions. Such dose response characterizations are well known to one of ordinary skill in the art. In a further embodiment, the composition comprises a CB1 cannabinoid receptor antagonist. The amount or dosage of the CB1 antagonist compound, in some embodiments, is as described herein for the CB1 antagonist compositions of the invention which lack an the OEA-like agonist, OEA-like compound or OEA-like appetite reducing compound.

In another aspect the present invention provides a kit comprising a container containing one or more unit dosages of a CB1 cannabinoid antagonist in which the unit dosage amount of the antagonist would not be expected to significantly affect appetite or food intake and a second container containing a pharmaceutical composition comprising a unit dosage of the PPARα agonist (e.g., OEA-like agonist, OEA-like compound) Or OEA-like appetite reducing compound or FAAH inhibitor in an amount which by itself would not be expected to significantly affect appetite or food intake.

The CB1 cannabinoid receptor antagonist and the PPARα agonist (e.g., OEA-like agonist, OEA-like compound) Or OEA-like appetite reducing compound or FAAH inhibitor of the above compositions may be present as any of their pharmaceutically acceptable salts.

In each of these aspects, the compositions include, but are not limited to, compositions suitable for oral, rectal, topical, parenteral (including subcutaneous, intramuscular, and intravenous), pulmonary (nasal or buccal inhalation), or nasal administration, although the most suitable route in any given case will depend in part on the nature and severity of the conditions being treated and on the nature of the active ingredient. An exemplary route of administration is the oral route. The compositions may be conveniently presented in unit dosage form and prepared by any of the methods well-known in the art of pharmacy.

In practical use, the cannabinoid antagonists and the PPARα agonist (e.g., OEA-like agonist, OEA-like compound) Or OEA-like appetite reducing compound or FAAH inhibitor can be combined as the active ingredient(s) in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. The percentage of an active compound in these compositions may, of course, be varied and may conveniently be between about 2 percent to about 60 percent of the weight of the unit. The amount of the CB1 cannabinoid receptor antagonist and the PPARα agonist (e.g., OEA-like agonist, OEA-like compound) Or OEA-like appetite reducing compound in such therapeutically useful compositions is typically such that a synergistically effective dosage will be obtained when both active agents are administered to the same recipient. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. To prevent breakdown during transit through the upper portion of the GI tract, the composition may be an enteric coated formulation.

In some embodiments, the pharmaceutical composition or formulation has a FAAH inhibitor in place of the PPARα agonist (e.g., OEA-like agonist, OEA-like compound) Or OEA-like appetite reducing compound. Such compositions may further include the CB-1 cannabinoid receptor antagonist. Such inhibitors can increase the endogenous level of OEA so as to synergize with an administered CB1-cannabinoid receptor antagonist. In some embodiments, the composition includes a FAAH inhibitor with an OEA-like compound to increase the ability of the OEA-like compound to synergize with the CB-1 cannabinoid receptor antagonist.

C. Administration.

The cannabinoid receptor antagonists and the PPARα agonist (e.g., OEA-like agonist, OEA-like compound) Or OEA-like appetite reducing compound and compositions of the invention can be administered parenterally. Solutions or suspensions of the active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include, but are not limited to, sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The PPARα agonist (e.g., OEA-like agonist, OEA-like compound) Or OEA-like appetite reducing compound or FAAH inhibitor may be effective synergists of a CB1 cannabinoid receptor antagonist over a wide dosage range. For example, in the treatment of adult humans, the OEA-like appetite reducing compound may be dosages from about 10 to about 100 mg, about 100 to about 500 mg or about 1 to about 10 mg may be needed. The compositions of the invention can be effective over a wide dosage range as expressed in mg/kg dosages. For example, in the treatment of adult humans, dosages from about 10 to about 200 mg/kg, about 1 to about 10 mg/kg or about 10 to about 100 mg/kg may be needed. Doses of the 0.1 to about 1 mg/kg, and more preferably from about 0.01 to about 1 mg/kg, per day may be used. A most preferable dosage is about 0.1 mg to about 70 mg per day.

The cannabinoid antagonists of the invention may be effective synergists with the PPARα agonist (e.g., OEA-like agonist, OEA-like compound) Or OEA-like appetite reducing compound or FAAH inhibitor over a wide dosage range. For example, in the treatment of adult humans, dosages from about 10 to about 100 mg, about 100 to about 500 mg or about 1 to about 10 mg may be needed. The CB1 receptor antagonist compositions of the invention can be effective over a wide dosage range as expressed in mg/kg dosages. For example, in the treatment of adult humans, dosages from about 10 to about 200 mg/kg, about 1 to about 10 mg/kg or about 0.1 to about 1 mg/kg may be needed. Doses of the 0.05 to about 100 mg/kg, and more preferably from about 0.01 to about 10 mg/kg, per day may be used. A most preferable dosage is about 0.1 mg to about 70 mg per day.

The exact dosages of each active agent (e.g., the cannabinoid antagonist and the PPARα agonist (e.g., OEA-like agonist, OEA-like compound) Or OEA-like appetite reducing compound, or FAAH inhibitor) will depend upon the mode of administration, on the therapy desired, the form in which each active agent is administered, the subject to be treated and the body weight of the subject to be treated, and the preference and experience of the physician or veterinarian in charge.

Generally, the cannabinoid antagonist and the PPARα agonist (e.g., OEA-like agonist, OEA-like compound) Or OEA-like appetite reducing compound or FAAH inhibitor can be dispensed alone in unit dosage for separate administration or together in a unit dosage form. The unit doses may comprise preferably from about 0.1 to about 1000 mg of one or more of the active ingredients together with a pharmaceutically acceptable carrier per unit dosage. Usually, dosage forms suitable for oral, nasal, pulmonary or transdermal administration comprise from about 0.001 mg to about 100 mg, preferably from about 0.01 mg to about 50 mg of each active agent admixed with a pharmaceutically acceptable carrier or diluent. For storage and use, these preparations preferably contain a preservative to prevent the growth of microorganisms.

The synergy between the PPARα agonist (e.g., OEA-like agonist, OEA-like compound) Or OEA-like appetite reducing compound or FAAH inhibitor and the CB1-cannabinoid antagonist make it possible to eliminate or control or reduce the side effects associated with the use of these compounds to reduce appetite. In one embodiment, the preferred dosages of each agent are identified by first separately identifying the optimum dose levels for the individual OEA-like agonist, OEA-like compound or OEA-like appetite reducing compound and the individual CB1 cannabinoid receptor antagonist. The optimum dosage of the OEA-like agonist, OEA-like compound or OEA-like appetite reducing compound upon individual administration is then reduced by 10% to 20%, or from 20-40%, 40%-60%, 60%-80%, or 80% or greater to provide the OEA dosages for use according to the invention (e.g., in combination with the CB1 cannabinoid receptor antagonist). The optimum dosage of the CB1 cannabinoid receptor antagonist upon individual administration is then reduced by about 10% to 20%, or about 20-40%, about 40%-60%, 60%, 60%-80%, or 80% or greater to provide the OEA dosages for use according to the invention (e.g., in combination with the CB1 cannabinoid receptor antagonist).

In some embodiments, both the PPARα agonist (e.g., OEA-like agonist, OEA-like compound) Or OEA-like appetite reducing compound) Or FAAH inhibitor and the CB1 cannabinoid receptor dosages are reduced from their individual optimum dosages to the same extent (e.g., about 10%, about 20%, about 30%, about 40%, about 50%, about 60%, or about 70% or greater). In other embodiments, the dosages for the CB1 cannabinoid receptor antagonist and the OEA-like agonist, OEA-like compound or OEA-like appetite reducing compound or FAAH inhibitor are reduced by different percents of their individual optimum dosages. In one embodiment, an optimum dosage is the lowest dosage which provides a demonstrable reduction in appetite. In another embodiment, it is the dosage which provides one-half of the maximum effect of the drug on appetite.

Administration of an appropriate amount the compositions may be by any means known in the art such as, for example, oral or rectal, parenteral, intraperitoneal, intravenous, subcutaneous, subdermal, intranasal, or intramuscular. In some embodiments, administration is transdermal. An appropriate amount or dose of the candidate compound may be determined empirically as is known in the art. An appropriate or therapeutic amount is an amount sufficient to effect a loss of body fat or a loss in body weight in the animal over time. The compositions can be administered as often as required to effect a loss of body fat or loss in body weight, for example, hourly, every six, eight, twelve, or eighteen hours, daily, or weekly.

Formulations suitable for oral administration can consist of (a) liquid solutions, such as water, saline or PEG 400; (b) Capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The pharmaceutically or physiologically acceptable salts include, but not limited to, a metal salts such as sodium salt, potassium salt, lithium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

The present invention encompasses various isomers of respective compounds, prodrugs and the like.

Injection solutions and suspensions can be prepared from sterile powders, granules, and tablets of the kind previously described. Formulations suitable for parenteral administration, such as, for example, by intraarticular (in the joints), intravenous, intramuscular, intradermal, intraperitoneal, and subcutaneous routes, include, but are not limited to, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

With respect to transdermal routes of administration, methods for transdermal administration of drugs are disclosed in Remington's Pharmaceutical Sciences, 17th Edition, (Gennaro et al. Eds., Mack Publishing Co., 1985). Dermal or skin patches are a preferred means for transdermal delivery of the compounds of the invention. Patches preferably provide an absorption enhancer such as DMSO to increase the absorption of the compounds. Other methods for transdermal drug delivery are disclosed in U.S. Pat. Nos. 5,962,012, 6,261,595, and 6,261,595. Each of which is incorporated by reference in its entirety.

Preferred patches include, but are not limited to, those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin.

The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

The cannabinoid CB1 antagonists and the PPARα agonist (e.g., OEA-like agonist, OEA-like compound) Or OEA-like appetite reducing compound or FAAH inhibitors may be used in combination with still other compounds of the invention or with other drugs that may also be useful in dieting or the treatment, prevention, suppression or amelioration of body fat, or appetite, or treatment of an appetency disorder. Such other drugs, may be administered, by a route and in an amount commonly used therefor, contemporaneously or sequentially with a compound of the invention. When a compound of the invention is used contemporaneously with one or more other drugs, a pharmaceutical composition in unit dosage form containing such other drugs and the compound is preferred. When used in combination with one or more other active ingredients, the compound of the present invention and the other active ingredients may be used in lower doses than when each is used singly. Accordingly, the pharmaceutical compositions of the present invention include, but are not limited to, those that contain one or more other active ingredients, in addition to the compounds disclosed above.

The following examples are provided for illustrative purposes, and are not intended to limit the scope of the invention as claimed herein. Any variations in the exemplified articles and/or methods which occur to the skilled artisan are intended to fall within the scope of the present invention.

### EXAMPLES

### Example 1

### Synthesis of Fatty Acid Ethanolamide Compounds, Homologues and Analogs

Methods for the formation of fatty acid ethanolamines from ethanolamines and the corresponding fatty acyl are relatively straight forward and known to one of ordinary skill in the art. For example, fatty acid ethanolamides may be synthesized by reacting a fatty acid or fatty acid chloride with an aminoalcohol as described by Abadji et al. (Abadji, V., Lin, S. Y., Taha, G., Griffin, G., Stevenson, L. A., Pertwee, R. G. & Makriyannis, A. J. Med. Chem. 37, 1889-1893 (1994)). Fatty acids may be prepared similarly to the procedure of Serdarevich and Carroll (Serdarevich, B. & Carroll, K. K. J. Lipid Res. 7, 277-284 (1966)). Radioactively labeled fatty acid ethanolamides can be prepared by reaction with acyl chlorides (Nu-Check Prep, Elysian, Minn.) with [³H]ethanolamine (10-30 Ci/mmol; American Radiolabeled Chemicals, St. Louis) as described by Desarnaud, F., Cadas, H. & Piomelli, D. (1995) J. Biol. Chem. 270, 6030-6035. Compounds can be purified by flash column chromatography or HPLC. Compound identity can be established by use of NMR and/or gas chromatography-mass spectrometry and thin layer chromatography.

Starting reagents and materials may be purchased from Avanti Polar Lipids, Cayman Chemicals (Ann Arbor, Mich.), Nu-Check Prep, Research Biochemicals, or Sigma. Briefly, according to methods taught by Giuffrida, A., et al. (see, Giuffrida, et al., “Lipid Second Messengers” (ed. Laychock, S. G. & Rubin, R. P.) 113-133 (CRC Press LLC, Boca Raton, Fla., 1998)) and Devane, et al. (Devane, et al., Science, 258:1946-1949 (1992)), unlabeled or labeled fatty acyl ethanolamines can be synthesized by the reaction of the corresponding fatty acyl chlorides with unlabeled or labeled ethanolamine. The fatty acid chorides can be dissolved in dichloromethane (10 mg/ml) and reacted with ethanolamine at −0.4° C. for 15 minutes. The reaction can be quenched by the addition of purified water. After vigorous stirring the phases are allowed to separate. The upper aqueous phase can be discarded. The organic phase can be washed twice with water. These washes remove the unreacted ethanolamine. This method provides a quantitative formation of fatty acyl ethanolamines. The ethanolamines are concentrated to dryness under a stream of nitrogen gas and can be reconstituted in an organic solvent such as dichloromethane at a concentration of 20 mM. The resulting fatty acyl ethanolamine solution can be stored at −20° C. until needed for use.

The chemistry of fatty acid carboxylic acid groups, primary and secondary amines, and primary alcohol groups is well known to one of ordinary skill in the art. Fatty acid ethanolamides having a variety of substituents on the ethanolamine portion thereof can be formed in many ways, but most preferably by starting with the corresponding substituted ethanolamine and fatty acid moieties. Such substituted ethanolamines would include the alkyl aminoethanol ethers and acyl aminoethanol esters as well as secondary akyl ethanol amines. Alternatively, the particular fatty acid ethanolamide can be synthesized from the corresponding fatty acid ethanolamide by the addition of the appropriate substituent groups.

A. Synthesis of OEA.

Oleoylchloride can be purchased from Nu-Check Prep (Elysian, Minn.) Or prepared following standard procedures. Oleoylchloride can be dissolved in dichloromethane (10 mg/ml) and allowed to react with five equivalents of ethanolamine for 15 min. at 0-4° C. The reaction can be stopped by the addition of purified water. After vigorous stirring and phase separation, the upper aqueous phase can be discarded and the organic phase washed twice with water to remove non-reacted ethanolamine. The resulting OEA can be concentrated to dryness under a N₂ stream, reconstituted in chloroform at 20 mM, and stored at −20° C. until use.

### Example 2

### Test Methods, Physiology and Pharmacological Activity of OEA-Like Compounds and/or OEA-Like Modulators

Animals. Male Wistar rats (200-350 g) were used. Procedures should meet NIH guidelines detailed in the Guide for the Care and Use of Laboratory Animals, and the European Communities directive 86/609/EEC regulating animal research.

Chemicals. FAEs and [²H₄] FAEs were synthesized in the laboratory (Giuffrida, et al., “Lipid Second Messengers” (ed. Laychock, S. G. & Rubin, R. P.) 113-133 (CRC Press LLC, Boca Raton, Fla., 1998)); 1,2-dioleyl-sn-glycero-phosphoethanolamine-N-oleyl was purchased from Avanti Polar Lipids (Alabaster, Ala.); SR141716A was provided by RBI (Natick, Mass.) as part of the Chemical Synthesis Program of the NIMH (N01MH30003); SR144528 was a generous gift of Sanofi Recherche; all other drugs were from Tocris (Ballwin, Mo.) Or Sigma (Saint Louis, Mo.). FAE were dissolved in dimethylsulphoxide (DMSO) and administered in 70% DMSO in sterile saline (acute treatments) Or 5% Tween 80/5% propylenglycol in sterile saline (subchronic treatments) (1 ml per kg, i.p.). Capsaicin was administered in 10% Tween 80/10% ethanol/80% saline; SR141716A, SR144528, CCK-8 and CP-93129 in 5% Tween 80/5% propylenglycol/90% saline (1 ml per kg, i.p.).

Enzyme assays. In all biochemical experiments, rats were killed and tissues collected between 1400 and 1600 h, after varying periods of food deprivation. Microsome fractions were prepared as described (Désarnaud et al., J. Biol. Chem., 270:6030-6035 (1995)). NAT assays were performed using 1,2-di[¹⁴C]palmityl-sn-glycerophosphocholine as a substrate (108 mCi/mmol, Amersham, Piscataway, N.J.) (Cadas et al., H., J. Neurosci., 17:1226-1242 (1997)). FAAH assays were performed according to (Désarnaud et al., J. Biol. Chem., 270:6030-6035 (1995)), except that [³H]anandamide(arachidonyl-[1-³H]ethanolamide; 60 Ci/mmol; ARC, St. Louis, Mo.) was included as a substrate and radioactivity was measured in the aqueous phase after chloroform extraction.

HPLC/MS analyses. Plasma was prepared from blood obtained by cardiac puncture (Giuffrida, et al., Anal. Biochem., 280:87-93 (2000)) and CSF was collected from the cisterna magna using a 27G ½ needle (Precisionglide, USA). FAEs and NAPE were extracted from tissues with methanol/chloroform and fractionated by column chromatography (Giuffrida, et al., “Lipid Second Messengers” (ed. Laychock, S. G. & Rubin, R. P.) 113-133 (CRC Press LLC, Boca Raton, Fla., 1998)). FAEs were quantified by HPLC/MS, using an isotope dilution method (Giuffrida, et al., Anal. Biochem., 280:87-93 (2000)). Individual NAPE species were identified and quantified by HPLC/MS, using an external standard method (Calignano, et al., Nature, 408:96-101 (2000)).

Blood chemistry. Plasma β-hydroxybutyrate and glycerol were measured using commercial kits (Sigma, St. Louis, Mo.). Plasma prolactin, corticosterone and luteinizing hormone were quantified by radioimmunoassay (Navarro, et al., Neuroreport, 8:491-496 (1997)).

Feeding experiments. Acute experiments. Food intake was measured in 24-h food-deprived rats (Navarro, et al., J. Neurochem., 67:1982-1991 (1996)), administering drugs 15 min before food presentation. Subchronic experiments. Ad libitum fed rats received vehicle injections for three days. On day four, the animals were divided in two equal groups and gave them daily injections of vehicle or OEA (5 mg per kg at 1900 h) for 7 consecutive days, while measuring body weight, food intake and water intake.

Conditioned taste aversion. Rats were water-deprived for 24 h and then accustomed to drink from a graded bottle during a 30-min test period for four days. On day five, water was substituted with a 0.1% saccharin solution and, 30 min later, the animals received injections of vehicle, OEA (20 mg per kg) Or lithium chloride (0.4 M, 7.5 ml per kg). During the following two days, water consumption was recorded over 30-min test periods. The animals were then presented with water or saccharin, and drinking measured.

Operant responses for food. Rats were trained to lever press for food on a fixed ratio 1 (FR1) schedule of reinforcement, while food-restricted at 20 g of chow per rat per day (Rodriguez de Fonseca, et al., Acta Pharmacol. Sin., 20:1109-1114 (1999)). Once stable responding was achieved, the animals were trained to acquire an FR5, time out 2-min schedule of food reinforcement and kept in limited access to food. When a stable baseline was obtained, the animals were used to test the effects of vehicle or OEA (1, 5 or 20 mg per kg) administered 15 min before lever presentation. Test duration was 60 min.

Other behavioral assays. The elevated plus maze test was conducted as described (Navarro, et al., Neuroreport, 8:491-496 (1997)) after the administration of vehicle or OEA (20 mg per kg, i.p.). Horizontal activity in an open field (Beltramo et al., J. Neurosci., 20:3401-3407 (2000)) and pain threshold in the hot plate test (55° C.) (Beltramo et al., Science, 277:1094-1097 (1997)) were measured 15 min after injection of vehicle or OEA (20 mg per kg). Rectal temperature was measured using a digital thermometer (Martin-Calderón et al., Eur. J. Pharmacol., 344:77-86. (1998)).

In situ hybridization. Rats were accustomed to the handling and injection procedure for five days. On day six, vehicle or drug OEA (10 mg per kg, i.p.), or oleic acid (10 mg per kg) was administered, and the rats killed 60 min later by decapitation under anesthesia. In situ hybridization analyses were conducted using ³⁵S-labeled cRNA probes for c-fos (Guthrie et al., Proc. Natl. Acad. Sci. U.S.A., 90:3329-3333 (1993)) and choline acetyl transferase (ChAT) (Lauterborn et al., Brain Res. Mol. Brain Res., 17:59-69 (1993)). Average hybridization densities were determined from at least three tissue sections per rat. Statistical significance was evaluated using one-way analysis of variance (ANOVA) followed by the Tukey-Kramer post-hoc test for paired comparisons.

Data analysis. Results are expressed as mean±s.e.m of n separate experiments. The significance of differences among groups was evaluated using ANOVA followed by a Student-Newman-Keuls post hoc test, unless indicated otherwise.

A. Effects of Starvation on OEA and Other FAE Levels in the Rat.

In one embodiment, the invention provides methods of treatment wherein individuals needing to lose weight and/or body fat are tested for OEA levels before and/or during fasting. Individuals with low levels of OEA prior to or in response to fasting are particularly then targeted for OEA treatment.

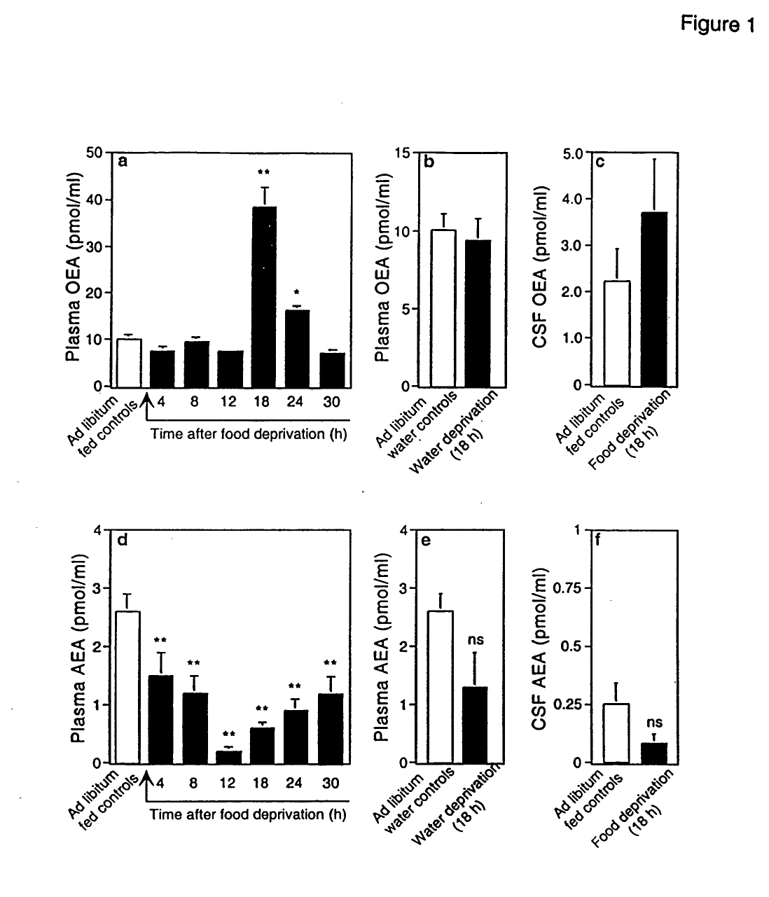

Rats were deprived of food while periodically measuring FAE levels in cardiac blood by high-performance liquid chromatography (HPLC) Coupled to electrospray mass spectrometry (MS). Plasma OEA remained at baseline levels for the first 12 h of fasting, markedly increased at 18-24 h, and returned to normal at 30 h (FIG. 1a). No such effect was observed following water deprivation (FIG. 1b) Or application of stressors such as restraint immobilization and lipopolysaccharide (LPS) administration [in pmol per ml; 10.3±0.8; 60 min after a 15-min immobilization, 8.4±1.6; 60 min after LPS injection (1 mg per kg), 7.0±0.7; n=6-9]. Plasma PEA was not significantly affected by any of these treatments (data not shown), whereas anandamide decreased rapidly upon food removal, remaining lower than baseline for the entire duration of the experiment (FIG. 1d). Anandamide levels also declined after immobilization (in pmol per ml; control, 3.6±0.4; immobilization, 1.1±0.5; n=7-8; P<0.01), LPS treatment (control, 2.0±0.5; LPS, 0.2±0.2; n=6; P<0.01) and, though not significantly, water deprivation (FIG. 1e). These results indicate that circulating OEA levels increase transiently during starvation. This response is selective for OEA over anandamide and other FAEs, and coincides temporally with the rise in blood glycerol and β-hydroxybutyrate (Table 1), which signals the shift of energy metabolism from carbohydrates to fatty acids as primary fuel (Cahill, G. F., Clin. Endocrinol. Metab., 5:397-415 (1976)).

TABLE 1
Plasma level of β-hydroxybutyrate (β-HBA) and glycerol in
fasting rats.

| | β-HBA | Glycerol |
|---|---|---|
| Free feeding | 1.2 ± 0.4 | 4.6 ± 0.9 |
| 2 h fasted | 1.2 ± 0.2 | 5.3 ± 0.6 |
| 4 h fasted | 0.8 ± 0.1 | 9.1 ± 1.8 |
| 8 h fasted | 1.3 ± 0.2 | 6.3 ± 0.4 |
| 12 h fasted | 4.6 ± 0.8* | 7.6 ± 1.0 |
| 18 h fasted | 6.8 ± 0.4* | 8.4 ± 0.4* |
| 24 h fasted | 9.1 ± 1.2* | 8.4 ± 0.3* |

Concentrations are expressed in mg per dl.
*P < 0.05, n = 3 per group.

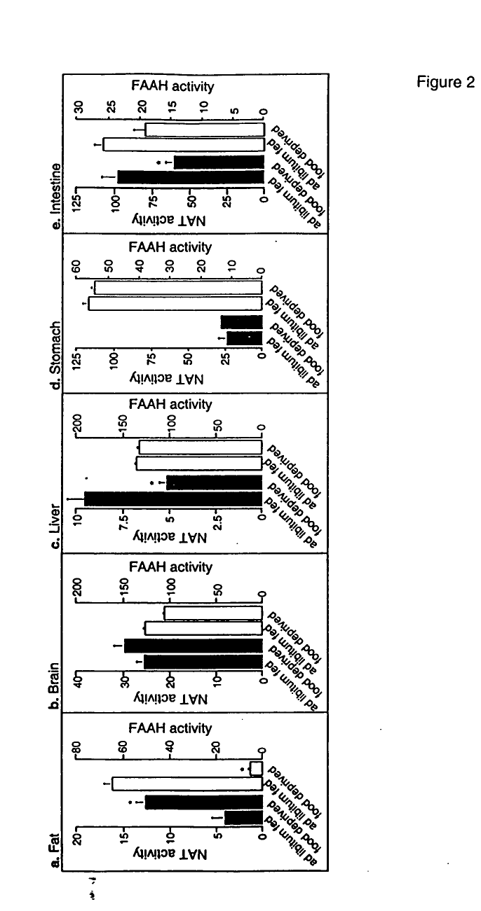

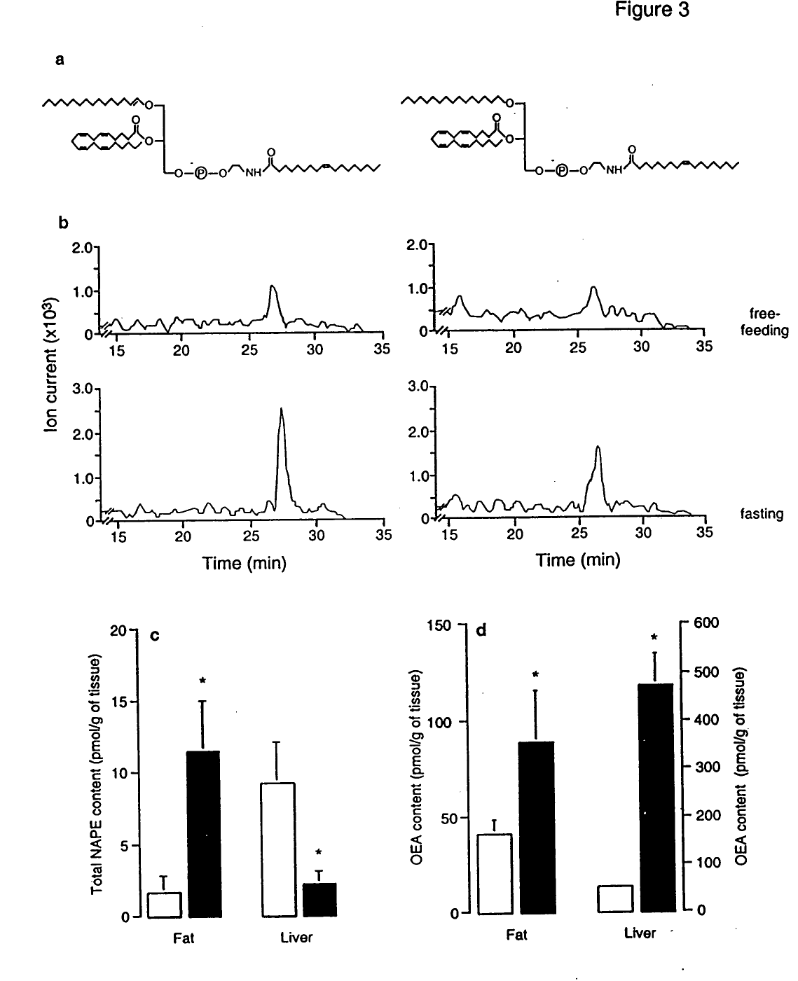

OEA levels in cerebrospinal fluid were not significantly affected by food deprivation (FIG. 1c), implying that the surge in plasma OEA may originate outside the CNS. To test this hypothesis, the impact of starvation on OEA metabolism in various rat tissues was investigated. The biochemical route by which animal cells produce and degrade OEA and other FAEs is thought to comprise three key enzymatic steps. Calcium ion-stimulated NAT activity transfers a fatty acid group from the sn-1 position of a donor phospholipid to the primary amine of phosphatidylethanolamine, producing NAPE2 (Schmid et al., Chem. Phys. Lipids, 80:133-142 (1996); Piomelli, et al., Neurobiol. Dis., 5:462-473 (1998)). Cleavage of the distal phosphodiester bond in NAPE by an unknown phospholipase D generates FAEs (Schmid, et al., Chem. Phys. Lipids, 80:133-142 (1996); Piomelli, et al., Neurobiol. Dis., 5:462-473 (1998)), which are eventually broken down to fatty acid and ethanolamine by an intracellular fatty acid amide hydrolase (FAAH) (Schmid, et al., J. Biol. Chem., 260:14145-14149 (1985); Cravatt, et al., Nature, 384:83-87 (1996)). Food deprivation (18 h) was accompanied by a marked increase in NAT activity in white adipose tissue (FIG. 2a), but not in the brain, stomach or kidney (FIG. 2b,d and data not shown). In liver, intestines and skeletal muscle, NAT activity was reduced by fast (FIG. 2c,d and data not shown). These enzymatic changes were paralleled by corresponding alterations in NAPE tissue content. Several molecular species of NAPE are present in rat tissues, including the OEA precursors alk-1-palmitoenyl-2-arachidonyl-sn-glycero-phosphoethanolamine-N-oleyl (NAPE 1; FIG. 3a) and alk-1-palmityl-2-arachidonyl-sn-glycero-phosphoethanolamine-N-oleyl (NAPE 2; FIG. 3a); and the PEA precursor alk-1-palmityl-2-arachidonyl-sn-glycero-phosphoethanolamine-N-palmityl (not shown). In agreement with NAT activity measurements, food deprivation increased NAPE content in fat, and decreased it in liver (FIG. 3b,c).

Since NAPE biosynthesis and FAE formation are tightly coupled processes (Cadas et al., H., J. Neurosci., 17:1226-1242 (1997)), one might expect starvation to augment the levels of OEA and other FAEs in adipose, but not in other tissues. Accordingly, fat from starved rats contained more OEA and PEA than did fat from free-feeding controls (FIG. 3d and data not shown), whereas no such difference was seen in the brain, stomach, and intestines (data not shown). Contrary to our expectation, however, the liver content of OEA and PEA was also higher in food-deprived than in free-feeding rats (FIG. 3d and data not shown). This discordance may be due to an accumulation of FAEs by the liver, which is consistent with the postulated roles of this organ in FAE recapture and metabolism (Bachur et al., J. Biol. Chem., 240:1019-1024 (1965); Schmid et al., J. Biol. Chem., 260:14145-14149 (1985)).

The hydrolysis to fatty acid and ethanolamine, catalyzed by FAAH, is a key step in FAE degradation (Bachur, et al., J. Biol. Chem., 240:1019-1024 (1965); Schmid, et al., J. Biol. Chem., 260:14145-14149 (1985); Cravatt, et al., Nature, 384:83-87 (1996); Désarnaud, et al., J. Biol. Chem., 270:6030-6035 (1995)). Food deprivation profoundly reduced FAAH activity in adipose membranes, but had no effect on FAAH activity in the brain, liver, stomach, intestines, kidney and skeletal muscle (FIG. 2a-e and data not shown). Thus, food deprivation may increase the levels of OEA and other FAEs in white fat in two synergistic ways, which are mechanistically distinct from other reactions occurring during lipolysis: stimulation of NAT activity may lead to increase the biosynthesis of NAPE and FAEs, while inhibition of FAAH activity may prolong the life span of newly synthesized FAEs. Although several tissues may contribute to the normal levels of OEA in the bloodstream, the dynamic biochemical changes observed in fat underscore the crucial role of this tissue in generating OEA during starvation.

B. Suppression of Food Intake by OEA and other FAEs.

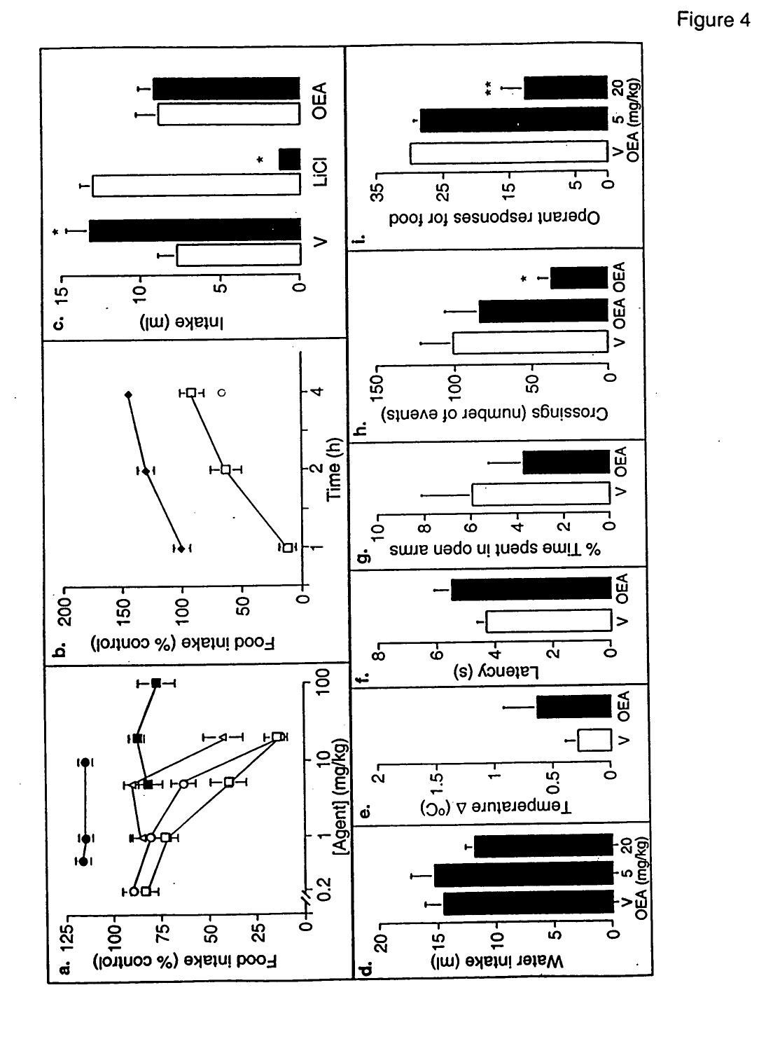

The effects of systemically administered OEA or an OEA-like compound or OEA-like modulator on food intake in rats can be assessed using a 24 h fast. In this system, OEA caused a dose- and time-dependent suppression of food intake (FIG. 4a,b) in rats given access to food after fasting. To define the selectivity of this response, various OEA analogs were evaluated for their ability to produce hypophagia.

Anandamide and oleic acid had no effect.

Palmitoylethanolamide was active but significantly less potent than OEA.

Elaidoylethanolamide (an unnatural OEA analog) was similar in potency to OEA (FIG. 4a).

These results indicate that OEA reduces eating in a structurally selective manner and that other fatty acid ethanolamide-like compounds can be identified for use according to the invention.

C. Specificity Over Cannabinoid Receptor Activators.

The molecular requisites for OEA hypophagia appear to be distinct from those involved in the interaction of anandamide with its known cannabinoid targets (Khanolkar et al., Life Sci., 65:607-616 (1999)). Cannabinoid receptor antagonists did not affect OEA hypophagia in vivo, and OEA did not displace cannabinoid binding to rat brain membranes in vitro. Thus, despite its structural and biogenetic relationships with anandamide, OEA acts differently and does not so depend on the endogenous cannabinoid system to produce anorexia.

D. Sustained Body Weight Reduction

In some embodiments, the OEA-like compounds and OEA-like modulators of the instant invention provide for a sustained fat reduction or body weight reduction upon prolonged administration to mammals. This effect can be advantageous as a variety of drugs suppress eating after acute administration, but fail to do so when treatment is prolonged (Blundell, J., Trends Pharmacol. Sci., 12:147-157 (1991)).

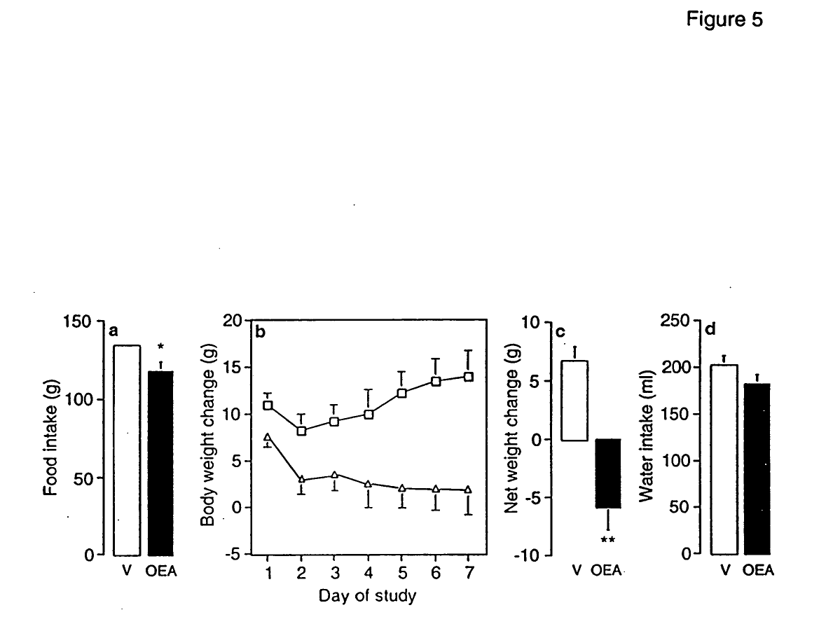

In this example, OEA was subchronically administered to rats. Daily injections of OEA (5 mg per kg, i.p.) for seven days resulted in a small, but significant decrease in cumulative food intake (FIG. 5a), which was accompanied by a profound inhibition of weight gain (FIG. 5b,c). OEA did not affect water intake (FIG. 5d). Without being wed to theory, the impact of OEA on body weight may only be partially explained by its moderate reduction of food consumption indicating that other factors, such as stimulation of energy expenditure or inhibition of energy accumulation, may contribute to this effect.

E. FAE's may have a Peripheral Site of Action.

In one of its aspects, the invention provides OEA-like compounds and OEA-like modulators having a peripheral site of action. Such a site can be advantageous in reducing the likelihood of central nervous system side effects.

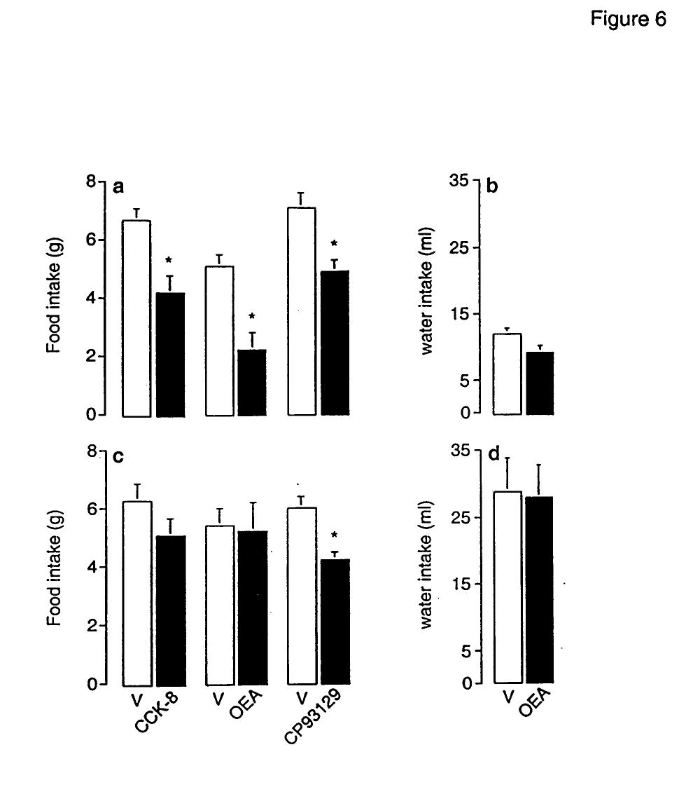

Though potent when administered peripherally, OEA was ineffective after direct injection into the brain ventricles (Table 2), suggesting that the primary sites of action of this compound might be located outside the CNS. As a further demonstration, sensory fibers in the vagus and other peripheral nerves were chemically destroyed by treating adult rats with the neurotoxin, capsaicin (Kaneko, et al., Am. J. Physiol., 275:G1056-G1062 (1998)). Capsaicin-treated rats failed to respond to peripherally administered cholecystokinin-8 (CCK-8) (FIG. 6a,c), drank more water than controls (FIG. 6b,d) and lost the corneal chemosensory reflex (data not shown), three indications that the neurotoxin had destroyed sensory afferents (MacLean, D. B., Regul. Pept., 11:321-333 (1985); Ritter et al., Am. J. Physiol., 248:R501-R504 (1985); Curtis et al., Am. J. Physiol., 272:R704-R709 (1997)). Treated animals also failed to respond to OEA (10 mg per kg, i.p.), but responded normally to the compound CP-93129, which targets 5-HT_{1B} receptors in the CNS (FIG. 6a,c) (Lee, et al., Psychopharmacology, 136:304-307 (1998)). Without being wed to theory, these findings support the hypothesis that OEA causes hypophagia by acting at a peripheral site, and that sensory fibers are required for this effect.

TABLE 2
Effects of intracerebroventricular OEA on food intake.

| | 60 min | 120 min | 240 min |
|---|---|---|---|
| vehicle | 5.8 ± 0.6 | 8.0 ± 0.5 | 9.5 ± 0.5 |
| OEA 0.4 μg | 4.8 ± 0.4 | 6.6 ± 0.4 | 8.4 ± 0.4 |
| OEA 2 μg | 4.9 ± 0.4 | 6.6 ± 0.6 | 8.7 ± 0.5 |
| OEA 10 μg | 5.9 ± 0.2 | 8.1 ± 0.4 | 9.6 ± 0.7 |

OEA(μg per animal) or vehicle (DMSO, 5 μl) was administered to 24 h food-deprived rats 15 min before food presentation. n = 12 per group.

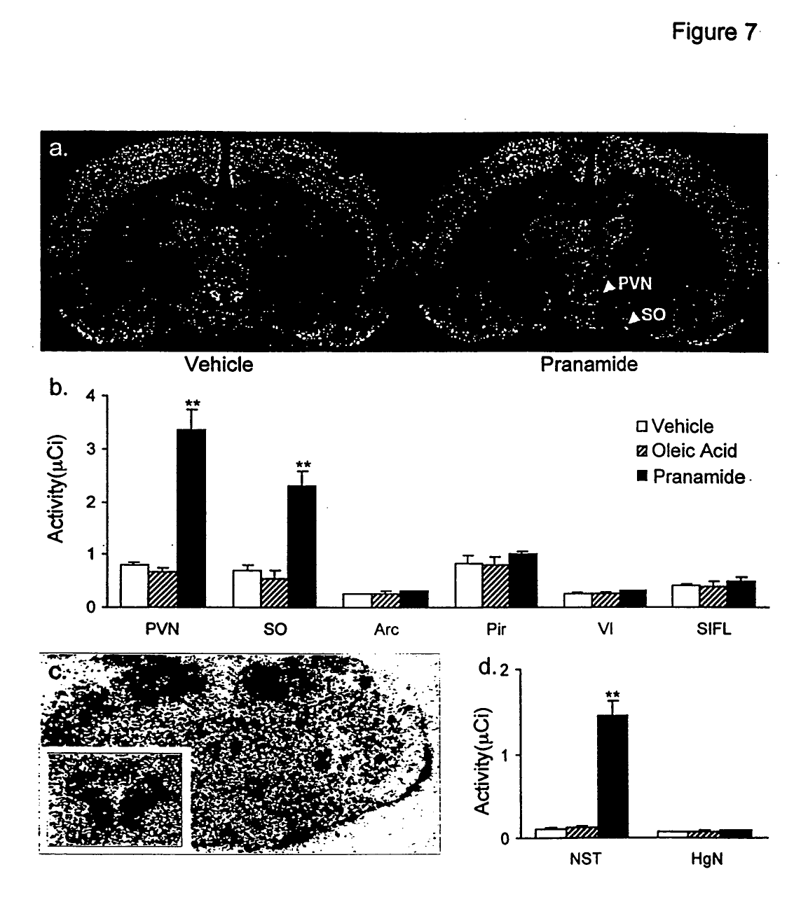

The compounds of the invention may use peripheral sensory inputs to suppress appetite. Peripheral sensory inputs related to appetite suppression recruit several CNS structures, which include the nucleus of the solitary tract (NST) in the brainstem and the arcuate and paraventricular (PVN) Nuclei in the hypothalamus (Schwartz et al., Nature, 404:661-671 (2000)). To identify the brain pathways engaged during OEA-induced hypophagia, mRNA levels for the activity regulated gene c-fos (Curran et al., Oncogene, 2:79-84 (1987)) were mapped by in situ hybridization after systemic administration of OEA, oleic acid or vehicle. When compared to controls, OEA (10 mg per kg, i.p.) evoked a highly localized increase in c-fos mRNA levels in the PVN, supraoptic nucleus (FIG. 7a) and NST (FIG. 7c). This enhancement was specific to these areas, insofar as c-fos expression in other brain regions was not significantly affected by OEA treatment (FIG. 7b,d). The finding that OEA stimulates c-fos mRNA expression in the NST (which processes vagal sensory inputs to the CNS) and the PVN (a primary site for the orchestration of central catabolic signals) (Schwartz et al., Nature, 404:661-671 (2000)), is consistent with a physiological role for this lipid as a peripheral mediator of anorexia.

OEA may reduce eating by inducing a non-specific state of behavioral suppression. If this is the case, OEA should cause conditioned taste aversion, which can be readily provoked in rats by a number of noxious substances (Green et al., Science, 173:749-751 (1971)), including lithium chloride (FIG. 4c). However, a maximal dose of OEA (20 mg per kg, i.p.) had little effect in this assay (FIG. 4c), suggesting that the compound may not be aversive. Several additional observations support the behavioral specificity of OEA. OEA did not alter water intake, body temperature, pain threshold (FIG. 4d-f), or activity of the hypothalamus-pituitary-adrenal (HPA) axis (Table 3). Moreover, OEA did not produce anxiety-like symptoms (FIG. 4g) and, though it reduced motor activity and operant responses for food, it did so at a dose that was substantially higher than those required to produce hypophagia (FIG. 4h-i). This pharmacological profile differentiates OEA from other appetite suppressants such as amphetamine and glucagon-like peptide 1 (whose effects often include aversion, hyperactivity, anxiety and activation of the HPA axis) and from the endogenous cannabinoid anandamide (which stimulates food intake in partially satiated animals, increases pain threshold, decreases body temperature and activates the HPA axis) (Pertwee, R. G., Exp. Opin. Invest. Drugs, 9:1553-1571 (2000)).

TABLE 3
Effects of OEA on plasma hormone levels.

| | B | PRL | LH |
|---|---|---|---|
| vehicle | 212 ± 24 | 10.8 ± 2.7 | 5.3 ± 0.9 |
| OEA 20 | 280 ± 61 | 8.2 ± 3.2 | 6.2 ± 1.5 |

In Table 2, plasma corticosterone (B), prolactin (PRL) and luteinizing hormone (LH) levels were measured by radioimmunoassay in plasma samples collected 60 min after injection of vehicle or OEA (prana, in mg per kg, i.p.) and are expressed in ng per ml. n = 6-9 per group.

OEA elicits hypophagia at physiologically relevant doses. 1 hr after administration of a half-maximally effective dose (5 mg per kg, i.p.), circulating OEA levels (16.1±2.6 pmol per ml) were significantly higher than baseline (10.1±1.1; P<0.05, Student's t test; n=5), but below those measured in 18-h food-deprived animals (FIG. 1a). Thus, the concentrations reached by OEA in blood during starvation can be sufficient to elicit notable behavioral responses.

F. Identifying Body Fat Reducing Compounds of the Invention.

The following illustrates how to identify appetite suppressors using OEA as a positive control. In particular, the measurement of body fat reduction and fatty acid oxidation are discussed.

The ability of an OEA-like compound or OEA-like modulator to reduce body fat can be evaluated by a number of methods. For example, appropriate amounts OEA and/or candidate compounds are administered to rats via intraperitoneal injection. The OEA and candidate compounds can be formulated in 70% DMSO in sterile saline, 5% Tween 80/5% polyethyleneglycol in sterile saline, or 10% Tween 80/10% ethanol/80% saline. Five mg per kg of OEA can be used as the positive control. Amounts of candidate compounds administered may range, for instance, from 1-25 mg per kg. Typically 1, 2, 5, 10, 15, and 20 mg per kg doses of each candidate compound can be administered to different sets of rats to determine which dose is optimal. Injections may be given 30 minutes before the animals' principal meal for 7-14 days.

The effect of the candidate compound on total body fat can be determined by taking direct measurements of the rat's body fat using skin fold calipers. Skin on the rats' backs, abdomen, chest, front and rear legs can be pinched with calipers to obtain measurements before administration of OEA and/or candidate compounds and every 48 hours during and after administration of OEA and/or candidate compounds. Differences in measurements in at least two of the pinched sites reflect the change in the rat's total body fat.

OEA-like compounds and modulators can be used to modulate fat metabolism. Such compounds can also be assayed for their effect on fatty acid metabolism. The effect of the candidate compound on fatty acid metabolism can be measured by measurements of fatty acid oxidation in primary cultures of liver cells. Hepatocytes may be used to determine the rate of oleate oxidation to ketone bodies and carbon dioxide. Such cells can be isolated from adult rat liver by enzymatic digestion as described by Beynen et al. in Diabetes 28:828 (1979). Cells typically are cultured in suspension and incubated in Krebs-Henseleit's bicarbonate medium supplemented with bovine serum albumin and glucose as described by Guzman & Geelen, Biochem. J., 287:487 (1992). The protein concentration of the cultured cells can be determined and cells seeded in 2 ml media so that 4-6 mg protein per ml is present in the reaction mixture. Cells can be incubated for 10 minutes at 37° C. with [¹⁴C]-oleic acid (Amersham), in the presence or absence of 10 μM OEA, reactions may be stopped with 200 μl 2M perchloric acid and acid-soluble products extracted with chloroform/methanol/water (5:1:1, vol:vol:vol). The aqueous phase can be removed and washed twice more. Protein concentration can be determined using a Lowry assay. The rate of oleate conversion into ketone bodies may be expressed as nmol of oleate oxidized per hour per mg protein and may be determined using liquid scintillation counting. Accordingly, OEA enhances oleate oxidation by 21+−6% (n=4, p<0.01 vs. control incubations by the Student t test).

G. Effect of OEA on Fatty Acid Metabolism.

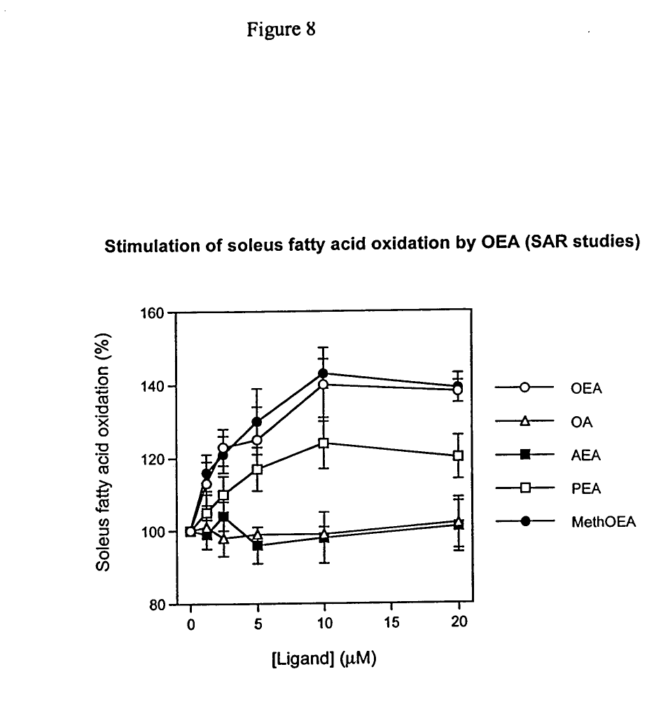

This example illustrates the effect of OEA on fat metabolism and methods for studying the same. Oleoylethanolamide (OEA) decreases body weight not only by suppressing appetite, but also by possibly enhancing body fat catabolism. The effects of OEA on fatty acid oxidation in major body-fat burning tissues (soleus muscle, liver, cultured cardiac myocytes and astrocytes) was examined. OEA significantly stimulates fatty acid oxidation in primary cultures of liver, skeletal muscle (soleus) and heart cells, whereas it has no effect in brain-derived astroglial cell cultures. In addition, OEA induces a significant mobilization of triacylglycerol stores from primary white adipose tissue cells. Table 4 details the methods and effects of OEA on fatty acid oxidation in these cells. Structure-activity relationship experiments provide evidence that the effect of OEA on skeletal muscle fatty acid oxidation is specific (FIG. 8). Thus, the effects of OEA are mimicked by the hydrolysis-resistant homologue methyl-OEA and -only partially by palmitoylethanolamide (PEA), but not by arachidonylethanolamide (AEA) Or oleic acid (OA). In short, these results show that lipid oxidation and mobilization are enhanced by OEA, and that the effects of OEA are restricted to peripheral sites.

TABLE 4

| Cell/tissue | Hepatocyte | Soleus muscle | Cardiomyocyte | Astrocyte | Adipocyte |
|---|---|---|---|---|---|
| Origin | Adult rat liver | Adult rat hind | Newborn rat | Newborn rat | Adult rat |
| | | limb | heart | brain cortex | epididymus |
| Isolation | Enzymatic | Dissection | Enzymatic | Enzymatic | Enzymatic |
| procedure | digestion | (Chiasson, | digestion (Flink | digestion | digestion |
| | (Beynen et al., | 1980) | et al., 1992) | (McCarthy & | (Rodbell, |
| | 1979) | | | De Vellis, | 1964) |
| | | | | 1980) | |
| Type of | Cell | Tissue | Cell monolayer | Cell | Cell |
| culture | suspension | suspension | | monolayer | suspension |
| Incubation | Krebs- | Krebs-Henseleit | High-glucose | Hams | Krebs- |
| medium | Henseleit | Hepes plus | DMEM plus | F12/DMEM | Henseleit |
| | bicarbonate | BSA and | BSA | plus insulin, | Hepes plus |
| | plus BSA and | glucose | (Wu et al., | transferrin, | BSA and |
| | glucose | (Fruebis et al., | 2000) | progesterone, | glucose |
| | (Guzman & | 2001) | | putrescine | (Rodbell, |
| | Geelen, 1992) | | | and selenite | 1965) |
| | | | | (Blazquez et | |
| | | | | al., 1998) | |
| Metabolic | [¹⁴C]oleate | [¹⁴C]oleate | [¹⁴C]oleate | [¹⁴C]oleate | Lypolysis |
| parameter | oxidation to | oxidation to | oxidation to | oxidation to | (glycerol |
| | ketone bodies | CO₂ (Fruebis et | CO₂ (Blazquez | ketone bodies | release) |
| | (Guzman & | al., 2001) | et al., 1998) | (Blazquez et | (Serradeil- |
| | Geelen, 1992) | | | al., 1998) | Le Gal et |
| | | | | | al., 2000) |
| Incubation | 10 | 30 | 30 | 30 | 30 |
| time (min) | | | | | |
| Stimulatory | 21 ± 6 (n = 4) | 36 ± 10 (n = 4) | 37 ± 9 (n = 3) | 2 ± 6 (n = 3) | 38 ± 16 (n = 3) |
| effect of 10 μM | | | | | |
| OEA | | | | | |
| (%) | | | | | |
| Statistical | P < 0.01 | P < 0.01 | P < 0.01 | Non | P < 0.01 |
| significance | | | | significant | |
| vs. control | | | | | |

References cited: Beynen A C et al., Diabetes 28:828-835 (1979); Blazquez C et al., J Neurochem 71:1597-1606 (1998); Chiasson R B “Laboratory Anatomy of the White Rat” WCB, Dubuque, Iowa (1980); Funk I L, et al., J Biol Chem, 267:9917-9924 (1992); Fruebis J et al., Proc Natl Acad Sci USA 98:2005-2010 (2001); Guzman, et al., Biochem J, 287:487-492 (1992); McCarthy K D, et al., J. Cell. Biol., 85:890-902 (1980); Rodbell M, J. Biol. Chem., 239:375-380 (1964); Rodbell M, Ann NY Acad. Sci., 131:302-314 (1965); Serradeil-Le Gal C, et al., FEBS Left, 475:150-156 (2000); Wu W, et al., J. Biol. Chem., 275:40133-40119(2000).

H. Role of Endogenous OEA in the Intestines.

The impact of feeding on intestinal OEA biosynthesis was studied. High performance liquid chromatography/mass spectrometry analyses revealed that small intestinal tissue from free-feeding rats contains substantial amounts of OEA (354±86 pmol per g, n=3). Intestinal OEA levels were markedly decreased after food deprivation, but returned to baseline after refeeding. By contrast, no such changes were observed in stomach (in pmol per g; control, 210±20; starvation, 238±84; starvation/refeeding, 239±60, n=3). Variations in intestinal OEA levels were accompanied by parallel alterations in NAT activity, which participates in OEA formation, but not in fatty acid amide hydrolase activity, which catalyzes OEA hydrolysis. These findings suggest that starvation and feeding reciprocally regulate OEA biosynthesis in small intestine. In agreement with an intra-abdominal source of OEA, plasma OEA levels in starved rats were found to be higher in portal than in caval blood (in pmol per ml; porta, 14.6±1.8; cava, 10.3±2.8; n=5). The contribution of other intra-abdominal tissues to OEA formation cannot be excluded at present. These results suggest many interventions to utilize the OEA systems in feeding behavior. According to this model, food intake may stimulate NAT activity enhancing OEA biosynthesis in the small intestine and possibly other intra-abdominal tissues. Newly produced OEA may activate local, sensory fibers, which may in turn inhibit feeding by engaging brain structures such as the NST and PVN.

The above results for Example 2 reveal an unexpected role for OEA in the peripheral regulation of feeding, and provide a framework to develop novel medicines for reducing body weight or body fat, for preventing body weight gain or body fat increase, for suppressing appetite or reducing food seeking behavior, or food intake, and for the treating eating disorders, overweight, or obesity. These medicines would include not only OEA analogues and homologues but also agents which control OEA levels by acting upon the OEA formation and hydrolyzing systems and enzymes as disclosed above.

### Example 3

### PPAR Modulation by OEA-Like Compounds and OEA-Like Modulators: Methods, Physiology and Pharmacology

Chemicals

GW 7647 {2-(4-{2-[3-Cyclohexyl-1-(4-cyclohexyl-butyl)-ureido]-ethyl}-phenylsulfanyl)-2-methyl-propionic acid was synthesized as follows. Phenethylamine was reacted with 4-cyclohexyl-butyric acid in the presence of diisopropylcarbodiimide and hydroxybenzotriazole (HOBT) in CH₂Cl₂. The resulting amide was treated with chlorosulfonic acid and PCl₅ to obtain 4-[2-(4-Cyclohexyl-butyrylamino)-ethyl]-benzenesulfonyl chloride, which was reduced (zinc dust/NaOAc/Ac₂O/glacial AcOH), to give thioacetic acid S-{4-[2-(4-cyclohexyl-butyrylamino)-ethyl]-phenyl}ester, the reaction of which with 2-bromo-2-methyl-propionic acid tert-butyl ester under strong basic condition afforded 2-{4-[2-(4-cyclohexyl-butyrylamino)-ethyl]-phenylsulfanyl}-2-methyl-propionic acid tert-butyl ester. This intermediate was then used in the synthetic route reported by Brown et al (Brown et al., 2000), leading to the title compound.

GW501516 [2-Methyl-4-[4-methyl-2-(4-trifluoromethyl-phenyl)-thiazol-5-ylmethylsulfanyl]-phenoxy}-acetic acid was synthesized via basic hydrolysis of the corresponding ethyl ester, prepared by coupling 5-chloromethyl-4-methyl-2-(4-trifluoromethyl-phenyl)-thiazole with (4-mercapto-2-methyl-phenoxy)-acetic acid ethyl ester (Chao, et al., 2001). To prepare the latter, o-tolyloxy-acetic acid ethyl ester was treated with chlorosulfonic acid to give (4-chlorosulfonyl-2-methyl-phenoxy)-acetic acid ethyl ester (synthesized). Reduction to (4-acetylsulfanyl-2-methyl-phenoxy)-acetic acid ethyl ester (zinc dust/NaOAc/Ac₂O/glacial AcOH), followed by hydrolysis under mild basic conditions (pyrrolidine in ethanol) yielded the desired intermediate (4-mercapto-2-methyl-phenoxy)-acetic acid ethyl ester.

OEA and other fatty acid ethanolamides can be prepared as described in Giuffrida, et al., Anal Biochem., 280:87-93, (2000)). All other chemicals from Sigma (Saint Louis, Mo.) Or Tocris (Ballwin, Mo.).

Animals

Male C57BL/6J mice, homozygous mice deficient for PPARα (129S4/SvJae-PPARα α^{tm/Gonz}) and wild-type mice (129S1/SvlmJ) were purchased from the Jackson Laboratory. Male Zucker rats (7 weeks of age) were obtained from Charles River. Male Wistar rats (325±30 g) were from Charles River. Animals were maintained on a 12-h light/dark cycle (light off at 5:30 PM) with water and chow pellets (RMH 2500, Prolab) available ad libitum.

Transactivation Assays

Transactivator plasmids pFA-PPARα, pFA-PPARδ, pFA-PPARγ and pFA-RXR, which encoded for the DNA-binding domain (DBD) Of hPPARα [symbol] ((499-1404), hPPARδ (412-1320), hPPARγ (610-1434) and hRXR (402-1389) fused to the DNA-binding domain (residues 1-147) Of yeast GAL4 under control of the human cytomegalovirus (CMV) promoter were generated. The plasmids contained a neomycin-resistance gene to provide stable selection with G418 (200 μg-ml⁻¹; Calbiochem). The HeLa cells were cultured in Dulbecco's-modified Eagles's medium (DMEM) supplemented with fetal bovine serum (10%). The cells were transfected with Fugene 6 (3 μl, Roche) Containing the pFR-luc plasmid (1 μg, Stratagene). Eighteen hours following transfection, the culture media was replaced with supplemented DMEM containing hygromycin (100 μg-ml⁻¹, Calbiochem). After 4 weeks in culture, the surviving clones were isolated and analyzed by luciferase assay. The clonal cell line HLR was selected because it demonstrated the highest levels of luciferase activity and transfected it with transactivator plasmids to generate cell lines that also expressed the DNA-binding domain of PPARγ (HLR-α), PPARδ (HLR-δ), PPARγ (HLR-γ), and RXR (HLR-rxr). The cells were cultured in supplemented DMEM containing hygromycin and G418. For transactivation assays, cells were seeded in 6-well plates (50,000 cells per well) and incubated for 7 hours in supplemented DMEM containing hygromycin and G418, plus appropriate concentrations of test compounds. Dual-luciferase reporter assay system (Promega) and an MLX Microtiter® plate luminometer (Dynex) were used to determine luciferase activity in cell lysates.

RNA Isolation and cDNA Synthesis

Tissues were stored in RnaLater™ (Ambion), extracted total RNA with TRIzol™ (Invitrogen) and quantified it with Ribogreen™ (Molecular Probes). cDNA was synthesized by using SuperscriptII RNase H-reverse transcriptase (Invitrogen).

Polymerase Chain Reaction (PCR)

Reverse transcription of total RNA (2 μg) was performed using Oligo(dT)₁₂₋₁₈ primer (0.2 μg) for 50 min at 42° C. Real Time Quantitative (RTQ) PCR was conducted using an ABI PRISM 7700 sequence detection system (Applied Biosystems). Primer/probe sets were designed using the Primer Express™ software and gene sequences available from the Genebank™ database. Primers and fluorogenic probes were synthesized by TIB (Adelphia). The primer/probe sequences for the mouse genes were:

PPARα,
F: CTTCCCAAAGCTCCTTCAAAAA,
R: CTGCGCATGCTCCGTG,
P: TGGTGGACCTTCGGCAGCTGG;
PPARδ,
F: GATGACAGTGACCTGGCGCT,
R: AGGCCTGGCCGGTCTC,
P: TTCATCGCGGCCATCATTCTGTGT;
PPARγ,
F: AGTGGAGACCGCCCAGG,
R: GCAGCAGGTTGTCTTGGATGT,
P: TTGCTGAACGTGAAGCCCATCGA;
CD36,
F: CGGCGATGAGAAAGCAGAA,
R: CAACCAGGCCCAGGAGC,
P: TGTTCAGAAACCAAGTGACCGGGAAAATAA;
FATP,
F: GCACAGCAGGTACTACCGCA,
R: GGCGGCACGCATGC,
P: TGCTGCCTTTGGCCACCATTCCTA;
I-FABP,
F: TCACCATCACCTATGGACCCA;
R, TCCAGTTCGCACTCCTCCC;
P: AGTGGTCCGCAATGAGTTCACCCTG;
GAPDH,
F: TCACTGGCATGGCCTTCC,
R: GGCGGCACGTCAGATCC,
P: TTCCTACCCCCAATGTGTCCGTCG.

RNA levels were normalized by using glyceraldehyde 3-phosphate dehydrogenase (GAPDH) as an internal standard. mRNA levels were measured by generating six-point serial standard curves using mouse total RNA. Estimates of relative mRNA abundance (in arbitrary units) were made by using the C_{T} value (Schmittgen et al., 2000). Relative quantifications of RNAs of interest were made by using the 2^{ΔCT} formula, in which ΔC_{T} was calculated by subtracting the C_{T} value for GAPDH from the C_{T} value for the gene of interest. This formula was validated for each primer/probe set by using six-point serial standard curves.

Feeding Experiments

Acute experiments. Drugs or appropriate vehicles (saline, for CCK-8 and d-fenfluramine; dimethylsulfoxide/saline, 70/30, for all other agents; 4 ml-kg⁻¹; i.p.) were administered at 5:00-5:30 PM to free-feeding mice, which were habituated to the experimental setting. Vehicles exerted no significant effect on feeding. Food intake and feeding microstructure was continuously monitored for 12 h using an automated system (ScriPro Inc, NY) (Gaetani et al., 2003).

Subchronic experiments. Male wild-type and PPARα null mice were fed with a very high-fat diet (60 kcal % fat; D12492; Research Diets, NJ). After 7 weeks, body mass indices were 0.355±0.01 g-cm² for wild-type mice (n=13) and 0.408±0.01 g-cm² for PPARα null mice (n=15), indicating that the mice had become obese (Gregoire et al., 2002). The mice were divided into 4 groups (n=7-8 each), and treated them for 4 additional weeks with vehicle (saline/polyethylene glycol/Tween 80, 90/5/5; 1 ml-kg⁻¹) Or OEA (5 mg-kg⁻¹, once daily, i.p.). In a separate experiment, obese Zucker rats were treated for 2 weeks with vehicle or OEA (5 mg-kg⁻¹, once daily, i.p.), while maintaining them on a regular rodent chow (RMH 2500, Prolab). Food intake and body weight were measured daily. At the end of the experiments, the animals were fasted overnight, and tissues and blood samples collected for biochemical analyses.

Chronic experiment: In a separate experiment, we treated obese Zucker rats for 2 weeks with vehicle (saline/polyethylene glycol/Tween-80, 90/5/5; 1 ml kg⁻¹, once daily, i.p.) or OEA (5 mg kg⁻¹, once daily, i.p.), while maintaining them on a regular rodent chow (RMH 2500, Prolab). We measured food intake and body weight daily. At the end of the experiments, the animals were fasted overnight, and tissues and blood samples collected for biochemical analyses.

Biochemical Analyses

Lipids were extracted from mouse liver and epidydimal adipose tissue (Bligh and Dyer, 1959) and measured triglycerides with a commercial kit (Sigma). Serum lipids and glucose were measured with an automated Synchron LX® system (Beckman-Coulter).

A. PPAR Modulatory Activity of OEA.

The following example exemplifies, using OEA as a model compound, how PPAR binding of OEA-like compounds and OEA modulators can be determined and demonstrates the use of an OEA-like compound or OEA-like modulator as a selective high potency binding agonist of PPARα.

To test the possibility that OEA may interact with one or more members of this family of ligand-operated transcription factors (Desvergne, B. & Wahli, W., Endocr Rev, 20:649-88 (1999), Chawla, A., et al., Science, 294:1866-70 (2001), and Berger, J. & Moller, D. E., Annu Rev Med, 53:409-35 (2002)), modified HeLa cells, which cannot metabolize OEA and other fatty acid ethanolamides (FAE) (Day et al., 2001), were genetically modified to stably express a luciferase reporter gene along with the ligand-binding domain of human PPARα, PPARδ, PPARγ, or retinoid X receptor (RXR) fused to the yeast GAL4 DNA-binding domain (Lazennec et al., 2000). In standard transactivation assays, each of these cell lines responded to appropriate synthetic PPARα agonists (data not shown).

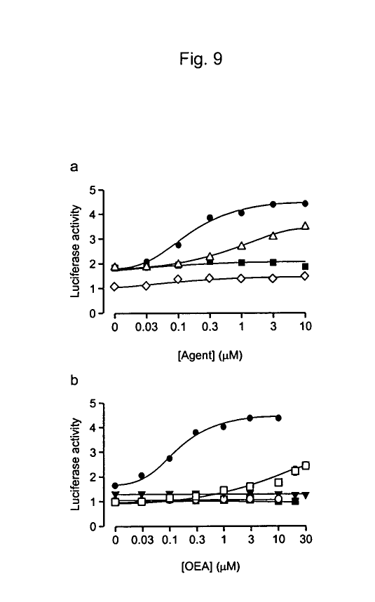

OEA caused a potent activation of PPAR, which was half-maximal at a concentration (EC₅₀) Of 120±1 nM (mean±s.e.m., n=16) (FIG. 9A). The compound also activated PPARβ, but less potently than it did PPARα (EC₅₀=1.1±0.1 μM) and had no effect on PPARγ or RXR (FIG. 9A). To explore the structural selectivity of this response, several analogs of OEA were tested for the ability to interact with PPARα. As previously reported (Göttlicher, et al., 1992; Kliewer, et al., PNAS, 91:7355 (1994)); Forman, et al., PNAS, 94:4312 (1997)) the parent fatty acid, oleic acid, activated PPARα with micromolar potency (EC₅₀=10.3±0.21 μM; n=16) (FIG. 9B). Conversely, stearylethanolamide, an FAE that contains the same number of carbon atoms as OEA but no double bonds, did not elicit a response (FIG. 9B). Equally ineffective were myristylethanolamide and the endogenous cannabinoid anandamide (arachidonylethanolamide) (Devane, et al., Science, 258:1946-9 (1992)) (FIG. 9B). Under the same conditions, the synthetic agonists Wy-14643 (Willson, et al., J. Med. Chem., 43:527-550 (2000)) and GW7647 (Brown, P. J., et al. in PCT Int. Appl. 32 (2000)) activated PPARα with EC₅₀ values of 1.4±0.1 μM and 150±20 nM, respectively (mean±s.e.m., n=5). The results suggest that OEA activates PPARα in vitro with high potency and selectivity.

B. PPARα Activation and OEA Anorexia.

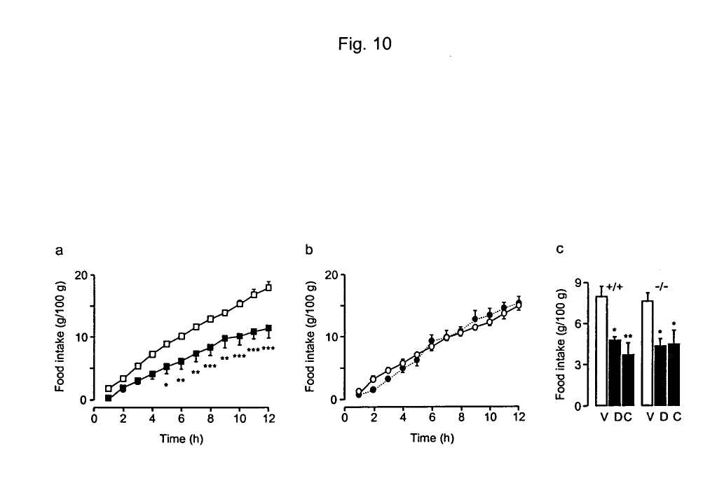

This example illustrates the use of PPARα-null mice to study whether an effect of an OEA-like compound is mediated by the PPARα receptor. To test whether PPARα activation contributes to the anorexiant properties of OEA, mutant mice were used in which the ligand-binding domain of PPARα had been disrupted by homologous recombination (Lee S S, et al., Mol. Cell Biol., June; 15(6):3012-3022 (1995)). Homozygous PPARα-null mice are fertile and viable, but do not respond to PPARα agonists and develop late-onset obesity (Lee S S, et al., Mol. Cell Biol., June;15(6):3012-3022 (1995); Butler and Cone, Trends Genet., October;17(10):S50-54 (2001)). Administration of OEA (10 mg-kg⁻¹, intraperitoneal, i.p.) reduced feeding in wild-type mice (FIG. 10A). This effect was absent in PPARα-deficient animals (FIG. 10B), which displayed OEA drug levels (Table 5) Comparable to those of wild-type controls and responded normally, however, to the serotonergic anorexiant d-fenfluramine and the peptide hormone cholecystokinin-octapeptide (CCK-8)(FIG. 10C). The effect of OEA was absent in PPAR-α-deficient animals (FIG. 2b), which displayed OEA drug levels comparable to those of wild-type controls (Supplementary Table 1) and responded normally to the serotonergic anorexiant d-fenfluramine and the peptide hormone cholecystokinin-octapeptide (CCK-8) (FIG. 2c).

TABLE 5
OEA levels in the liver of wild-type and PPAR-α^{-/-} mice.

| | Vehicle | OEA |
|---|---|---|
| Wild-type | 72.8 ± 12.1 | 150.5 ± 19.7 |
| PPAR-a-null | 73.5 ± 1.6 | 251.2 ± 28.2 |

OEA (5 mg kg⁻¹, i.p.) or vehicle was administered by i.p. injection. Liver OEA content was measured 1 h after administration by HPLC/MS. Results are expressed in pmol-g⁻¹ and are the mean ± sem of n = 3-4.

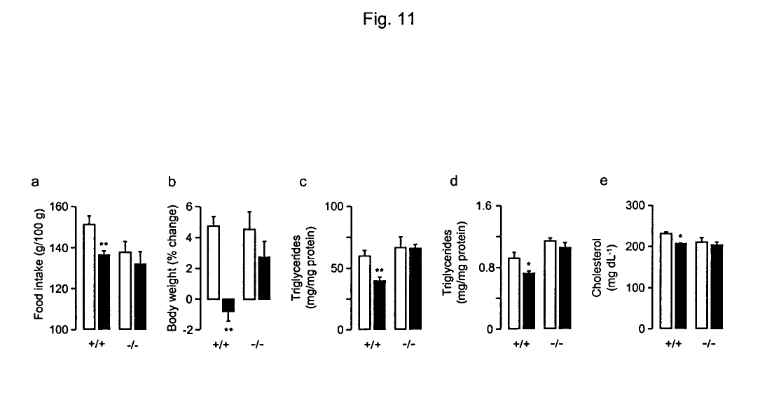

To determine the role of PPARα on the effect of subchronically administered OEA in rats in producing a sustained inhibition of food intake and inhibition of body-weight gain, wild-type and PPARα deficient mice were fed with a high-fat chow for 7 weeks to induce obesity, and treated them for 4 subsequent weeks with daily injections of vehicle or OEA (5 mg-kg⁻¹, i.p.). In obese wild-type mice, OEA significantly reduced cumulative food intake (normalized for body mass) (FIG. 11A) and suppressed body-weight gain (FIG. 11B). By contrast, no such effect was observed in obese PPARα deficient animals (FIG. 11A-B). These results suggest that expression of a functional PPARα is necessary for the satiety-inducing and weight-reducing actions of OEA. They also illustrate the use of PPAR-α null mammals to determine the receptor mechanism of an OEA-like compound.

C. High Potency Selective PPARα Agonist Compounds are Required to Affect Appetite and Body Weight Gain.

This example illustrates the screening of and use of compounds which are high affinity agonists for use in treating anorexia and to reduce body weight or body fat. The possibility that OEA modulates feeding through direct activation of PPARα was further investigated despite the fact that this possibility seemed negated by the fact that fibric acids, a class of PPARα agonists that is widely used in the therapy of hyperlipidemias, do not notably affect food intake (Best and Jenkins, Expert Opin Investig Drugs, 10:1901-11 (2001)). Fibric acids are, however, 200 to 900 times less potent than OEA at activating PPARα (Willson, T. M., et al., J. Med. Chem., 43:527-550 (2000)).

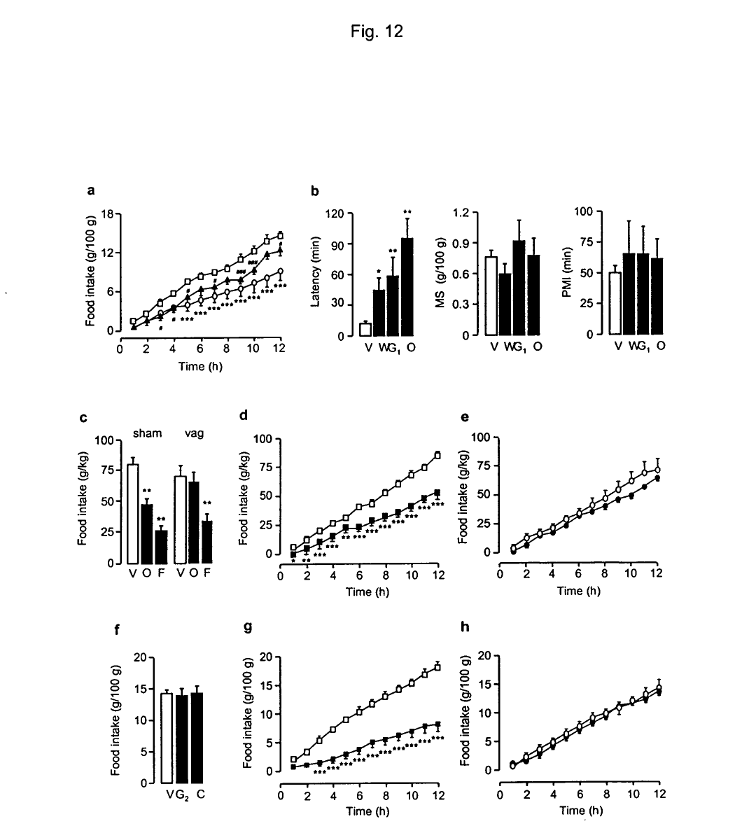

Therefore, to assess the contribution of PPAR-α to feeding regulation, compounds with potencies comparable to that of OEA were used: Wy-14643 (see, Willson, T. M., et al., J. Med. Chem., 43:527-550 (2000)) and GW7647 (see, Brown, P. J., et al. in PCT Int. Appl. 32 (2000)). Both drugs inhibited food intake in C57BL/6J mice (FIG. 12a), whereas the fibric acid derivative clofibrate did not (25-100 mg kg⁻¹; data not shown). Meal pattern analyses revealed that the anorexiant effects of Wy-14643 and GW7647 were due to a selective prolongation of eating latency rather than to changes in meal size or post-meal interval (FIG. 12b). This response is essentially identical to that elicited by OEA (10 mg kg⁻¹, i.p.) (FIG. 12b) and is suggestive of a satiety-inducing action.

OEA is thought to produce satiety by activating visceral sensory fibres (see, Rodriguez de Fonseca, et al., Nature 414, 209-12. (2001). Accordingly, in rats in which these fibres had been removed either by severing the vagus nerve below the diaphragm or by capsaicin treatment, OEA (10 mg kg⁻¹, i.p.) had no effect on food intake (FIG. 12c). These procedures also prevented the hypophagic effects of Wy-14643 (40 mg kg⁻¹, i.p.) (FIG. 12d-e and Table 6), but not those of the centrally acting anorexiant d-fenfluramine (FIG. 12c)

TABLE 6
Effects of sensory deafferentation on the anorexiant responses
to Wy-14643.

| | | | |
|---|---|---|---|
| 30 min | 60 min | 120 min | 240 min |

Control rats

| | | | | |
|---|---|---|---|---|
| Vehicle | 5.6 ± 0.8 | 6.5 ± 0.9 | 7.6 ± 0.8 | 10.2 ± 0.9 |
| Wy-14643 | 2.3 ± 1.3* | 3.6 ± 1.2* | 5.8 ± 1.3* | 6.4 ± 1.8* |

Capsaicin-treated rats

| Vehicle | 2.9 ± 0.9 | 4.5 ± 0.9 | 6.7 ± 0.8 | 8.8 ± 0.8 |
|---|---|---|---|---|
| Wy-14643 | 2.2 ± 1.2 | 3.7 ± 1.6 | 4.9 ± 1.5 | 7.8 ± 1.9 |
| Wy-14643 (40 mg kg⁻¹, i.p.) or vehicle was administered to 24-h food-deprived Wistar rats (325 ± 30 g) and food intake was measured manually. Results are the mean ± sem of n = 6. Asterisk, P < 0.05 vs vehicle. Capsaicin deafferentation. Male Wistar rats were treated with capsaicin or vehicle, as described¹. The animals were habituated to | | | | |
| # handling, food-deprived for 24 h and given Wy-14643 or vehicle (DMSO/saline, 70/30). Food pellets and spillage were measured manually 30-240 min after drug injection. | | | | |

The close correspondence between the effects of OEA and those of synthetic PPAR-α agonists suggests that OEA modulates feeding through activation of PPAR-α. This conclusion is reinforced by two findings. First, potent agonists at PPAR-β/δ (GW501516; 1-10 mg kg⁻¹, i.p.) (see, Oliver, W. R., Jr., et al., Proc Natl Acad Sci USA, 98:5306-11 (2001)) and PPAR-γ (ciglitazone; 15 mg kg⁻¹, i.p.) (see, Chang, A. Y., et al., Diabetes, 32:830-8 (1983)) did not affect feeding in C57BL/6J mice (FIG. 3f); and, second, mice deficient in PPAR-α did not respond to Wy-14643 (40 mg kg⁻¹, i.p.) (FIG. 3g-h). OEA has slight PPARβ activity. As the PPAR-β/δ agonist GW501516 does not affect food intake, and OEA does not induce satiety or weight reduction in PPAR-α null mice, the data indicate that the any role of PPAR-β/δ in OEA signalling is, if any, distinct from that of PPAR-α.

D. OEA Initiation of PPARα Gene Expression.

The above result was unexpected, because the actions of PPARα were thought to be mediated through transcriptional regulation of gene expression (Desvergne, B. & Wahli, W., Endocr Rev, 20:649-88 (1999), Chawla, A., et al., Science, 294:1866-70 (2001), and Berger, J. & Moller, D. E., Annu Rev Med, 53:409-35 (2002)), which was considered too slow to account for the rapid satiety-inducing effects of OEA.

Therefore, to further test the hypothesis that OEA activates PPARα, the ability of the compound to initiate expression of PPARα-regulated genes was investigated first, on the small intestine, which is one of the most likely sites of action of OEA (see, Rodriguez de Fonseca, et al., Nature 414:209-12 (2001)) and contains high levels of PPARα (see, Escher, et al., Endocrinology, 142:4195-4202 (2001)).

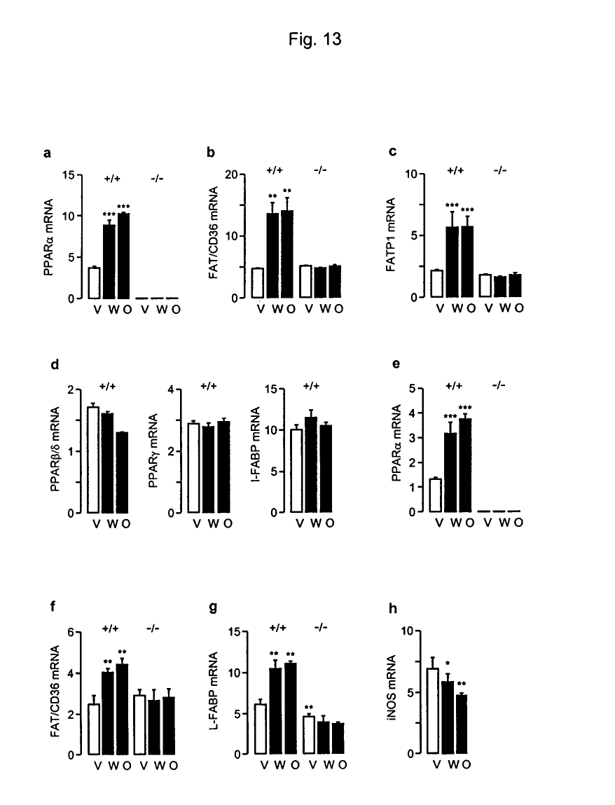

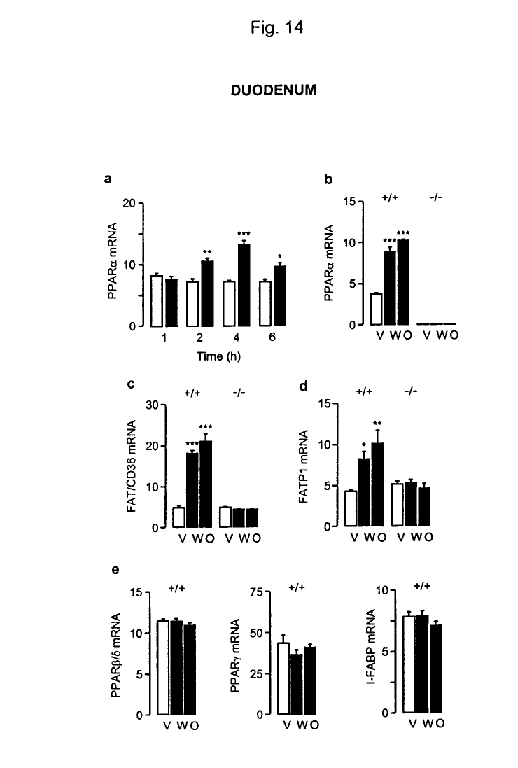

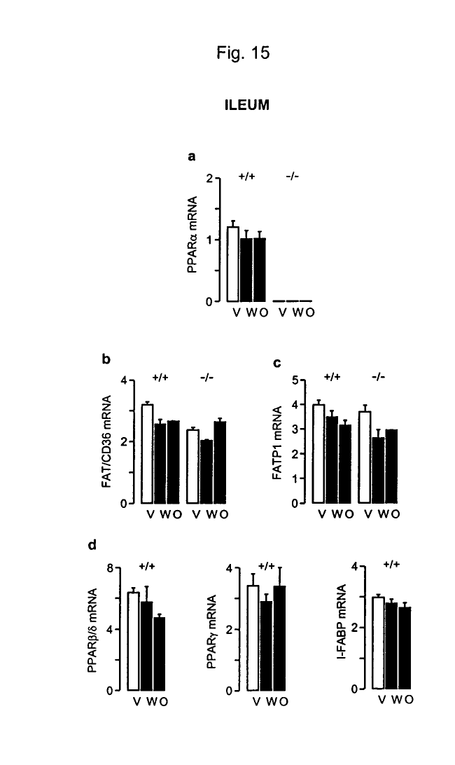

In the jejunum of wild-type mice, OEA (10 mg kg⁻¹, i.p.), but not oleic acid (10 mg kg⁻¹, i.p.; data not shown), increased the expression of three PPAR-α-regulated genes: PPAR-α itself (FIG. 13a), fatty acid translocase (FAT/CD36) (FIG. 13b) and fatty acid transport protein 1 (FATP1) (FIG. 13c) (see, Martin, G., et al., J. Biol. Chem., 272:28210-7 (1997) and Motojima, K., et al., J. Biol. Chem., 273, 16710-4 (1998)). Interestingly, a similar stimulatory effect was observed in the duodenum (FIG. 14) which, like the jejunum, plays a key role in fatty acid absorption, but not in the ileum (FIG. 15), which is primarily involved in the absorption of cholesterol and bile salts. By contrast, the expression of three related genes, which are not under the control of PPAR-α (intestinal fatty acid-binding protein, I-FABP, PPAR-β/δ and PPAR-γ) was not affected by OEA either in wild-type (FIG. 13d) Or PPAR-α-null mice (data not shown). Underscoring the role of PPAR-α in these responses, it was found that (i) the PPAR-α agonist Wy-14643 (30 mg kg⁻¹, i.p.) mimicked the effects of OEA (FIG. 13a-d), and (ii) OEA and Wy-14643 did not stimulate gene expression in mice deficient in PPAR-α (FIG. 13a-c). The ability of OEA to activate PPAR-α-mediated gene expression was not restricted to the intestine, as the compound also initiated transcription of PPAR-α-regulated genes in the liver of wild-type, but not PPAR-α-null mice (FIG. 13e-g).

In addition to stimulating transcription, PPAR-α activation also is known to induce the transrepression of various genes, such as inducible nitric-oxide synthase (iNOS) (see, Colville-Nash, P. R., et al., J. Immunology, 161:978-984 (1998)). Accordingly, in the jejunum of C57BL/6J mice, administration of OEA (10 mg kg⁻¹, i.p.) Or Wy-14643 (30 mg kg⁻¹, i.p.) significantly decreased iNOS expression (FIG. 13h), whereas oleic acid (10 mg kg⁻¹, i.p.) was ineffective (data not shown). These results indicate that OEA closely mimics the genomic actions of PPAR-α agonists in a PPAR-α-dependent manner.

E. Effect of OEA on Serum Lipids.

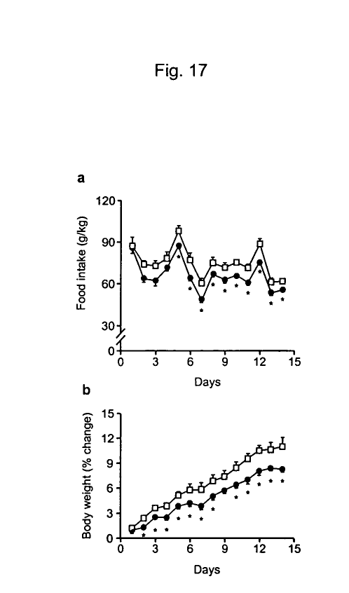

This example illustrates the use of an OEA-like compound to reduce serum lipids. If OEA enhances expression of PPARα-regulated genes, it also should reproduce the metabolic consequences of long-term treatment with PPARα agonists, a prominent example of which is the reduction of genetic or diet-induced hyperlipidemia (see, Best, J. D. & Jenkins, A. J., Expert Opin Investig Drugs, 10: 1901-11 (2001)). Consistent with this prediction, OEA treatment (5 mg-kg⁻¹, once daily for 2 weeks, i.p.) reduced fasting serum cholesterol and triglyceride levels in genetically obese Zucker (fa/fa) rats (Table 7). These effects were accompanied by a significant inhibition of food intake and body-weight gain (FIG. 17) and were qualitatively similar to those previously reported for the PPARα agonists clofibrate and fenofibrate (see, Cleary, et al. Atherosclerosis 66, 107-12. (1987) and Chaput, E., et al., Biochem Biophys Res Commun 271, 445-50. (2000)). Furthermore, high fat-fed wild-type and PPARα-null mice develop hypercholesterolemia, but maintain normal serum triglyceride levels (in mg-dl⁻¹; wild-type, cholesterol: 253±7; triglycerides: 72±3; PPARα-null, cholesterol: 216±11; triglycerides: 82±9 mg-dl⁻¹; n=8-9). A 4-week OEA regimen (5 mg-kg⁻¹, once daily, i.p.) partially corrected this alteration in wild-type mice, but was ineffective in PPARα-null animals (FIG. 11C). These findings indicate that long-term administration of OEA induces metabolic changes, which are reminiscent of those elicited by PPARα agonists and are abrogated by deletion of PPARα.

TABLE 7
Effects of OEA on serum lipids and glucose in obese Zucker rats.

| | Vehicle | OEA |
|---|---|---|
| Cholesterol | 99.88 ± 8.41 | 66.14 ± 7.06* |
| Triglycerides | 565.29 ± 55.50 | 394.17 ± 49.40* |
| Glucose | 229.29 ± 27.90 | 221.25 ± 23.80 |

OEA (5 mg kg−1, i.p.) or vehicle was administered once a day for 2 weeks. Serum cholesterol, triglycerides and glucose were measured and are expressed in mg dl−1. Results are the mean ± sem of n = 7-8. Asterisk, P < 0.05 vs vehicle.

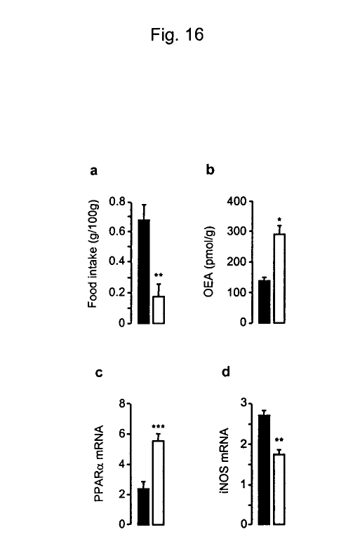

The ability of OEA to activate PPARα in vitro, the close similarity between its pharmacological properties and those of PPARα agonists, and the lack of such effects in PPARα null mice, indicate that OEA is a natural ligand for PPARα. The concerted regulation of OEA synthesis and PPAR-α/iNOS expression further supports this possibility. In the small intestine of C57BL/6J mice, OEA levels were significantly lower at night (1:30 AM), when the animals are actively engaged in feeding, than during the day (4:30 PM), when they are satiated and resting (FIG. 16a-b). Intestinal PPAR-α expression paralleled OEA levels (FIG. 16c), whereas expression of the PPAR-α transrepression target, iNOS, displayed an opposite pattern (FIG. 16d). Importantly, the diurnal concentrations of OEA in intestinal tissue (≈300 nM) were in the range needed to fully activate PPAR-α in vitro (EC₅₀=120 nM), suggesting that they may be adequate to engage this receptor and regulate transcription of its target genes in vivo.

In conclusion, these results indicate that OEA is the first natural compound that meets all key criteria for it to be considered an endogenous PPAR-α ligand: (i) it binds with nanomolar affinity to mouse and human PPAR-α; (ii) it mimics the actions of synthetic PPAR-α agonists in a PPAR-α-dependent manner; and (iii) it reaches, under appropriate physiological conditions, tissue levels that are sufficiently high to activate PPAR-α. Furthermore, the findings suggest that PPAR-α activation does not only mediate OEA-induced weight stabilisation, which is expected from the metabolic roles of this receptor (see, Desvergne, B. & Wahli, W., Endocr Rev, 20:649-88 (1999), Chawla, A., et al., Science, 294:1866-70 (2001), and Berger, J. & Moller, D. E., Annu Rev Med, 53:409-35 (2002)), but also is responsible for OEA-induced satiety, a behavioural role that was not previously attributed to PPAR-α. The molecular mechanism underlying this response is still undefined, but one possibility is that it may involve the regulation of intestinal NO production. Intestinal epithelial cells express the NO-synthesizng enzyme, iNOS, and generate significant amounts of this gaseous messager, which is thought to act as a peripheral orexigenic signal (see, Colville-Nash, P. R., et al., J. Immunology., 161:978-984 (1998), Sticker-Krongrad, et al., Life Sci., 58: PL9-15 (1996), and Janero, D. R., Nutrition, 17:896-903 (2001)). The ability of OEA to transrepress iNOS via PPAR-α suggests that iNOS down-regulation may contribute to the persistent anorexiant actions of OEA. Irrespective of these speculations, our study identifies OEA as a primary endogenous agonist for PPAR-α and opens new perspectives for the treatment of eating disorders.

### Example 4

### Methods for Identifying an OEA-Like Compound or an OEA-Like Modulator for Use in Modulating Appetite, Reducing Body Fat, or Regulating Fat Metabolism

An OEA-like compound or modulator for reducing body fat in a mammal can be identified by screening one or more OEA-like compounds or candidate OEA-like modulators in a binding or activation assay for each of PPARα, PPARβ and PPARγ and selecting the compound for further testing if it is a specific agonist of peroxisome proliferator activated receptor type a (PPARα) having at least a 5 fold specificity for PPARα over both PPARγ and PPARβ and produces a half-maximal effect on PPARα at a concentration of less than 1 micromolar; and then testing the compound selected in step (i) by administering the compounds to the mammal and determining, as compared to an appropriate vehicle control, the amount of body fat reduction, appetite suppression, or fat metabolism alteration.

### Example 5

### Exemplary FAAH Inhibitors for Use in Treating a Disease or Condition Mediated by PPARα or Responsive to Therapy by a PPARα Agonist

Trifluoroketone inhibitors such as the compound of Formula IX are also contemplated for use in inhibiting FAAH to raise endogenous levels of OEA or treat the subject conditions and disorders.

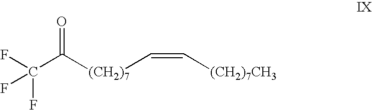

Such compounds are taught in U.S. Patent Application No. 6,096,784 herein incorporated by reference.

Other compounds for use according to the invention include octylsulfonyl and octylphosphonyl compounds. See, Quistand, et al., in Toxicology and Applied Pharmacology, 179:57-63 (2002). See also Quistand, et al., in Toxicology and Applied Pharmacology, 173:48-55 (2001).

Other compounds for use according to the invention include the alpha-keto-oxazolpyridines which are reversible and extremely potent inhibitiors of FAAH. See, Boger et al., PNAS USA, 97:5044-49 (2000). Suitable compounds include compounds of the Formula: 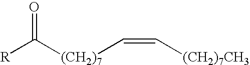
wherein R is an alpha-keto oxazolopyridinyl moiety such as

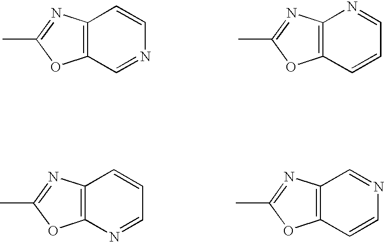

Boger et al. teach other suitable compounds for use according to the invention including substituted alpha-keto-heterocycle analogs of fatty acid amides. In particular, wherein R is an alpha-keto oxazolopyridinyl moiety and the fatty acid moiety is a homolog of oleic acid or arachidonic acid.

Other FAAH inhibitors for use according to the invention include fatty acid sulfonyl fluorides such as compound AM374 which irreversibly binds FAAH. See, Deutsch, et al., Biochem. Biophys Res Commun., 231:217-221 (1997).

Other preferred FAAH inhibitors include, but are not limited to, the carbamate FAAH inhibitors disclosed in Kathuria et al., Nat Med January;9(1):76-81(2003) incorporated herein by reference for the FAAH inhibitor compounds it discloses. Particularly preferred are selective FAAH inhibitors such as URB532 and URB597 disclosed therein.

### Example 6

### Methods of Screening Compounds for FAAH Inhibitory Activity

Methods for screening compounds for FAAH inhibitory activity in vitro are well known to one of ordinary skill in the art. Such methods are taught in Quistand, et al., in Toxicology and Applied Pharmacology, 179:57-63 (2002); Quistand, et al., in Toxicology and Applied Pharmacology, 173: 48-55 (2001); and Boger, et al., PNAS USA, 97:5044-49 (2000).

Methods for screening compounds for FAAH inhibitory activity in vivo and increased endogenous cannabinoid levels or activity are known to one of ordinary skill in the art. Such methods include measurement of fatty acid ethanolamides in tissue and are taught in Quistand, et al., in Toxicology and Applied Pharmacology, 179: 57-63 (2002); Quistand, et al., in Toxicology and Applied Pharmacology, 173: 48-55 (2001); Boger, et al., PNAS USA, 97:5044-49 (2000). See, U.S. Pat. No. 6,096,784. See also PCT Publication WO 98/24396. See, Cravatt, et al., PNAS, 98:9371-9376 (2001).

### Example 7

### Exemplary OEA-Like Compounds and/or OEA-Like Modulators

In some embodiments, specific PPARα agonists are used to modulate appetite or reduce body fat or to alter fat metabolism. Selective high affinity PPARα agonists are well known in the art. Exemplary OEA-like modulators include GW 7647 and GW501516. PPARα modulators are taught in U.S. Pat. No. 6,468,996; U.S. Pat. No. 6,465,497; U.S. Pat. No. 6,534,517; U.S. Pat. No. 6,506,781; U.S. Pat. No. 6,407,127; and U.S. Pat. No. 6,200,998. The disclosures of each of which are herein incorporated by reference with particular respect to the subject matter of the PPAR modulatory compounds they disclose and only to the extent not inconsistent with the present specification. Specific PPAR agonists can be ascertained by use of a PPAR activation assay panel of PPARα, PPARγ, and PPARβ.

### Example 8

### Effects of CB1 Cannabinoid Receptor Antagonists on Appetite and the Synergism Between CB1 Cannabinoid Receptor Antagonists and OEA-Like Appetite Reducing Compounds

Animals

Male Wistar rats (350±50 g) were housed individually with food and water available ad libitum, except when restriction was required. All animal procedures met the National Institutes of Health guidelines for the care and use of laboratory animals, and the European Communities directive 86/609/EEC regulating animal research.

Surgery

For intracerebroventricular (i.c.v.) injections, stainless steel guide cannulae aimed at the lateral ventricle were implanted in rats. The animals were anesthetized with equithesin and placed in a Kopf stereotaxic instrument with the incisor bar set at 5 mm above the interaural line. A guide cannula (7 mm, 23 gauge) was secured to the skull by using two stainless steel screws and dental cement, and closed with 30 gauge obturators (Navarro, et al., J. Neurochem, 67:1982-1991 (1996); Rodriguez de Fonseca, et al., Nature, 414:209-212 (2001)). The implantation coordinates were 0.6 mm posterior to bregma, ±2.0 mm lateral, and 3.2 mm below the surface of the skull. These coordinates placed the cannula 1 mm above the ventricle. After a 7-day post surgical recovery period, cannula Patency was confirmed by gravity flow of isotonic saline through an 8 mm-long 30-gauge injector inserted within the guide to 1 mm beyond its tip. This procedure allowed the animals to become familiar with the injection technique.

Chemicals

Capsaicin was purchased from Sigma (St. Louis, Mo., USA), and cholecystokinin octapeptide sulphated (CCK-8), WIN 55,212-2 and CP93129 from Tocris Cookson Inc. (UK). SR141716A ([N-piperidino-5-(4-chlorophenyl)-1-(2,4-dichlorophenyl)-4-methylpyrazole-3-carboxamide]) was a gift of Sanofi Recherche (Montpellier, France). Anandamide and oleoylethanolamide (OEA) were synthesized in the laboratory (Giuffrida, et al., Anal Biochem, 280:87-93 (2000)). Capsaicin was dissolved in 10% Tween 80, 5% propylenglycol and 90% saline. All other drugs were dissolved in dimethylsulphoxide (DMSO) and administered in 70% DMSO in sterile saline.

High-Performance Liquid Chromatography/Mass Spectrometry (HPLC/MS) Analyses.

Anandamide was solvent-extracted from tissues, fractionated by column chromatography and quantified by HPLC/MS with an isotope dilution method, as described (Giuffrida, et al., Anal Biochem., 280:87-93 (2000)).

Drug Treatments.

Capsaicin was administered subcutaneously (12.5 mg/ml, Kaneko, et al., Am. J. Physiol., 275:G1056-G1062 (1998)) in rats anesthetized with ethyl ether. The total dose of capsaicin (125 mg/kg) was divided into three injections (25 mg/kg in the morning and 50 mg/kg in the afternoon of the first day, 50 mg/kg on the 2nd day). Control rats received vehicle injections. Experiments were performed 10 days after capsaicin treatment in rats that 1) had lost the corneal chemosensory reflex (eye wiping for 1-3 min following application of 0.1% ammonium hydroxide into one eye); and 2) showed enhanced water intake 10 days after capsaicin treatment. Water intake (in ml/4 hr) was: vehicle 13.6±1.4; capsaicin rats 24.0±1.9, P<0.01 (n=12).

Drugs were administered by i.p. injection 15 min prior to food presentation in a volume of 1 ml/kg. For i.c.v. administration the obturator was removed from the guide cannula and an 8-mm injector (30-gauge stainless steel tubing) that was connected to 70 cm of calibrated polyethylene-10 tubing was lowered into the ventricle. The tubing was then raised until flow began and 5 μl of drug solution were infused over a 30-60 s period. The injector was left in the guide cannula for additional 30 s and then removed. The stylet was immediately replaced. Animals were tested 5 min after injections. The i.c.v. cannula placements were evaluated after each experiment by dye injection. Only those rats with proper i.c.v. placements were included in the data analysis.

Food Intake Studies.

The effects of drugs on feeding behavior were analyzed in 24 h food-deprived animals, which had been habituated to handling (Navarro, et al., J. Neurochem., 67:1982-1991 (1996); Rodríguez de Fonseca, et al., Nature, 414:209-212 (2001)), or in partially satiated animals (i.e., 24 h food-deprived animals allowed to eat for 60 minutes prior to drug testing, Williams, et al., Physiol Behav., 65:343-6 (1998)). To this end, 48 h before testing, the bedding material was removed from the cage and a small can containing food pellets was placed inside the cage for 4 h. The animals were then food-deprived for 24 h, with free access to water. 15 min after drug administration, the animals were returned to their home cage, where a can with a measured amount of food (usually 30-40 g) a bottle containing 250 ml of fresh water were placed. Food pellets and food spillage were weighed at 60,120 and 240 min after starting the test, and the amount of food eaten was recorded. At the end of the test, the amount of water consumed was also measured. For partial satiation of animals, 24 h food-deprived rats were allowed to eat from the can during 1 h. The can was retired and intake was recorded. 15 min after drug injections, the food was again presented, and the amount consumed was recorded hourly for the following 4 h.

Open Field Test.

Exploratory behavior in the open field was studied in an opaque open field (100×100×40 cm) as described previously (Rodríguez de Fonseca, et al., Nature, 414:209-212 (2001)). Rats were habituated to the field for 10 min the day before testing. On the experimental day, the animals were placed in the center of the field and locomotor activity (number of lines crossed) and exploratory behavior (number of rearings and time spent in the center of the field) were scored for 5 min. All the experiments were performed 60 min after drug injections, and behavior was scored by trained observers blind to experimental conditions.

Statistics.

Statistical significance was assessed by one-way or multifactorial ANOVA, as required. Following a significant F value, post hoc analysis (Student-Newman-Keuls) was performed.

### Results

Effects of Feeding on Anandamide Levels.

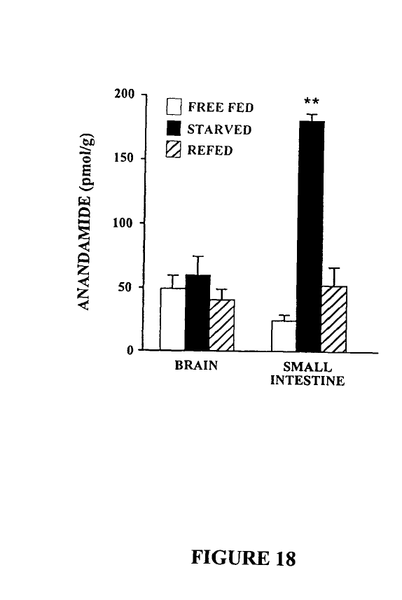

The effects of starvation and refeeding on anandamide content was investigated in intestinal tissue, where various intrinsic signals modulating food intake, such as CCK (Reidelberger, Am. J. Physiol., 263:R1354-R1358 (1992)) and OEA (Rodríguez de Fonseca, et al., Nature, 414:209-212 (2001)), are generated. As shown in FIG. 18, food deprivation (24 h) was accompanied by a 7-fold increase in anandamide content in the small intestine, an effect that was reversed upon refeeding. By contrast, no such increase was observed in brain or stomach tissues (FIG. 18 and data not shown). The change in intestinal anandamide did not result from inhibition of anandamide degradation. Indeed, fatty acid amidohydrolase (FAAH) activity, which catalyzes the deactivating hydrolysis of anandamide, was not affected by the feeding status (data not shown).

Central Cannabinoid Administration Does not Affect Food Intake.

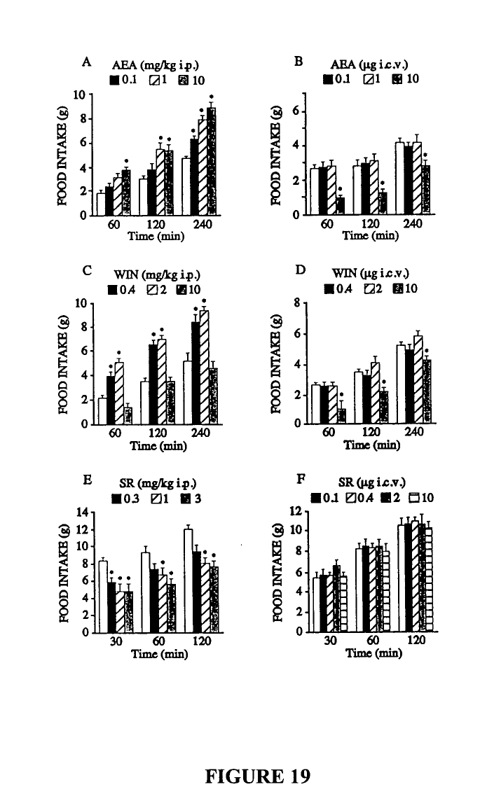

As previously reported (Williams, et al., Physiol. Behav., 65:343-6 (1998)), systemic (i.p.) administrations of the endogenous cannabinoid anandamide or the synthetic cannabinoid agonist WIN 55,212-2 (0.1-2 mg/kg) had no effect on food intake in food deprived rats (data not shown). Nevertheless, when administered to partially satiated animals, these drugs elicited significant and prolonged hyperphagia (FIGS. 19A and 19C). At a dose of 10 mg/kg, WIN 55,212-2 also produced profound immobility, which interfered with feeding behavior (FIG. 19C). By contrast, central injections of anandamide and WIN 55,212-2 had no effect on feeding, except at the highest dose (10 μg), which resulted in motor impairment (FIGS. 19B and 19D and data not shown).

Following systemic administration, the selective CB1 antagonist SR141716A elicited a dose-dependent reduction of food intake in both 24 h food-deprived rats (FIG. 19E) and partially satiated rats (data not shown). However, the drug had no effect following central administration (FIG. 19F). Irrespective of the administration route, SR141716A reduced exploratory behavior in the open field, indicating that the drug effectively interacted with brain cannabinoid receptors (Navarro, et al., Neuroreport, 8:491-496 (1997)). In support of this conclusion, the rearing frequency after SR141716A administration was (in number of events per 5 min) 1) i.p. vehicle 17.9±2.3; 2) i.p. SR141716A (3 mg/kg) 9.4±2.0, (P<0.05); 3) i.c.v. vehicle 16.6±3.1; 4) i.c.v. SR141716A (10 μg) 4.9±1.1, (P<0.05). The results indicate that the hyperphagia evoked by cannabinoid receptor agonists, as well as the anorexia elicited by the CB1 antagonist SR141716A are dependent on the interaction of these agents with peripheral cannabinoid receptors.

Sensory Deafferentation Prevents Cannabinoid Effects on Feeding.

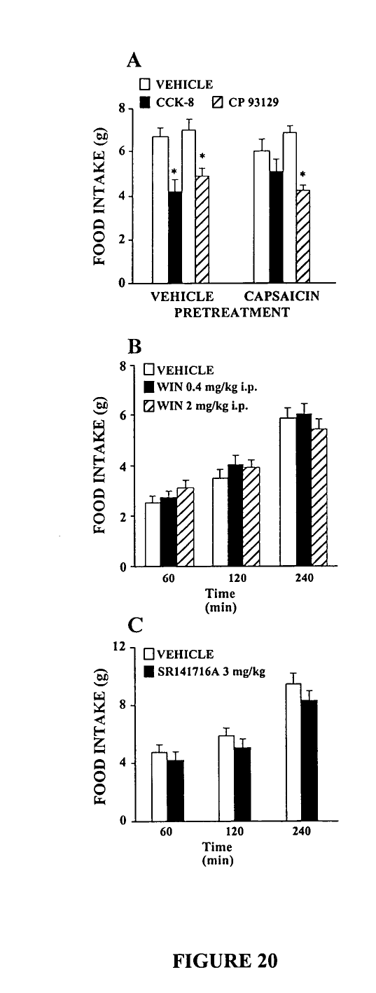

Treatment with the neurotoxin capsaicin abolished the anorexic response elicited by the peptide CCK-8 (10 μg/kg i.p.), but not that induced by the centrally acting 5HT-1B agonist CP 93129 (1 mg/kg, i.p. FIG. 20A) indicating that sensory terminals innervating the gut had been destroyed. The treatment also resulted in a loss of the hyperphagic effects of either WIN 55,212-2 (2 mg/kg, i.p., FIG. 20B) Or anandamide (2 mg/kg i.p., data not shown) and of the hypophagic effects of SR141716A (3 mg/kg, i.p.) (FIG. 20C).

The present results suggest, first, that systemically administered cannabinoid agents (both agonists and antagonists) affect food intake predominantly by engaging peripheral CB1 receptors localized to capsaicin-sensitive sensory terminals; and, second, that intestinal anandamide is a relevant signal for the regulation of feeding.

The concentration of anandamide in intestinal tissue increases during food deprivation, reaching levels that are 3 fold greater than those needed to half maximally activate CB1 receptors (Devane, et al., Science, 258:1946-9 (1992)). This surge in anandamide levels, the mechanism of which is unknown, may serve as a short-range hunger signal to promote feeding. This idea is supported by the ability of SR141716A to reduce food intake after systemic, but not central administration. Locally produced anandamide also may be involved in the regulation of gastric emptying and intestinal peristalsis, two processes that are inhibited by this endocannabinoid (Calignano, et al., Eur. J. Pharmacol., 340:R7-8 (1997); Izzo, et al., Naunyn Schmiedebergs Arch Pharmacol., 360:221-3 (1999)). Thus, intestinal anandamide appears to serve as an integrative signal that concomitantly regulates coordinating food intake and gastrointestinal motility.

CB1 Cannabinoid Receptor Antagonists and OEA-Like Appetite Reducing Compounds Synergistically Inhibit Feeding.

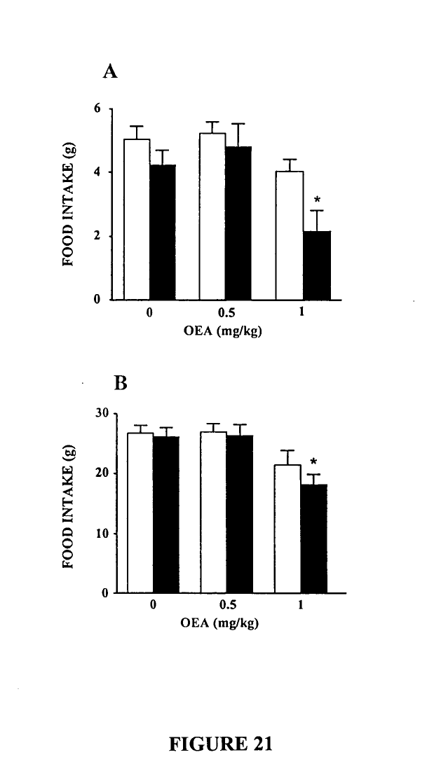

The small intestine produces both anandamide, which stimulates food intake (Williams and Kirkham, Psychopharmacology, 143:315-7 (1999)), and OEA, which inhibits it by acting on peripheral sensory fibers (Rodriguez de Fonseca, et al., Nature, 414:209-212 (2001)). The possible interaction of these fatty acid ethanolamides on feeding, was examined. Whether blockade of CB1 receptors with a low, subthreshold dose of SR141716A, an exemplary CB1 cannabinoid receptor antagonist, potentiates the inhibitory actions of OEA on food intake was studied. The results, illustrated in FIG. 21, indicate that SR141716A and OEA act synergistically to decrease eating in food-deprived animals. The effects were observed 120 min after the injection of OEA (FIG. 21A) and lasted for at least 24 hr (FIG. 21B).

All publications and Patent applications and references cited in this specification are herein incorporated by reference to the extent not inconsistent with the present disclosure as if each individual publication or Patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of reducing food consumption in a mammal, said method comprising administering to said mammal a first compound which is a PPARα agonist and a second compound which is an antagonist of the CB1 cannabinoid receptor, whereby the consumption of food by the animal is reduced.

2. The method according to claim 1, wherein the PPARα agonist is an OEA-like agonist.

3. The method of claim 1, wherein the PPARα agonist is oleoylethanolamide, palmitoylethanolamide or elaidoylethanolamide.

4. The method of claim 1, wherein the antagonist is a pharmaceutically acceptable salt or solvate of a compound of the formula: 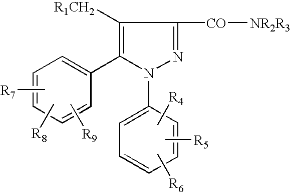
wherein R₁ is hydrogen, a fluorine, a hydroxyl, a (C₁-C₅)alkoxy, a (C₁-C₅)alkylthio, a hydroxy(C₁-C₅)alkoxy, a group —NR₁₀R₁₁, a cyano, a (C₁-C₅)alkylsulfonyl or a (C₁-C₅)alkylsulfinyl;
R₂ and R₃ are a (C₁-C₄)alkyl or, together with the nitrogen atom to which they are bonded, form a saturated or unsaturated 5- to 10-membered heterocyclic radical which is unsubstituted or monosubstituted or polysubstituted by a (C₁-C₃)alkyl or by a (C₁-C₃)alkoxy;
R₄, R₅, R₆, R₇, R₈ and R₉ are each independently hydrogen, a halogen or a trifluoromethyl, and if R₁ is a fluorine, R₄, R₅, R₆, R₇, R₈ and/or R₉ can also be a fluoromethyl, with the proviso that at least one of the substituents R₄ or R₇ is other than hydrogen; and
R₁₀ and R₁₁ are each independently hydrogen or a (C₁-C₅)alkyl, or R₁₀ and R₁₁, together with the nitrogen atom to which they are bonded, form a heterocyclic radical selected from pyrrolidin-1-yl, piperidin-1-yl, morpholin-4-yl and piperazin-1-yl, which is unsubstituted or substituted by a (C₁-C₄)alkyl.

5. The method of claim 4, wherein said antagonist is of the formula: 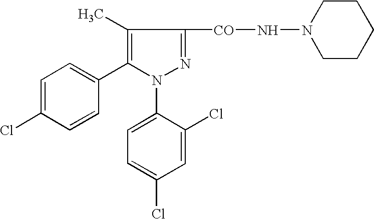
or a pharmaceutically acceptable salt thereof.

6. A method according to claim 1, wherein the mammal is human.

7. A method according to claim 6, wherein said human is overweight or obese.

8. A method according to claim 1, wherein the PPARα agonist is a compound of the following formula: 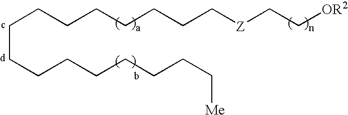
wherein n is any number from 0 to 5;
the sum of a and b can be any number from 0 to 4;
Z is a member selected from —C(O)N(R^{o})—; —(R^{o})NC(O)—; —OC(O)—; —(O)CO—; O; NR^{o}; and S, in which R¹ and R² are independently selected from the group consisting of substituted or unsubstituted alkyl, hydrogen, substituted or unsubstituted C₁-C₆ alkyl, substituted or unsubstituted lower (C₁-C₆) acyl, homoalkyl, and aryl;
up to eight hydrogen atoms of the compound may also be substituted by methyl group or a double bond; and
the molecular bond between carbons c and d may be unsaturated or saturated,
or a pharmaceutically acceptable salt thereof.

9. A method according to claim 1, wherein said PPARα agonist is administered with a pharmaceutically acceptable carrier by an oral, rectal, topical, or parenteral route.

10. A method according to claim 1, wherein said antagonist is administered with a pharmaceutically acceptable carrier by an oral, rectal, topical, or parenteral route.

11. A method according to claim 1, wherein said antagonist and said PPARα agonist are administered together.

12. A method according to claim 1, wherein said antagonist and said PPARα agonist are each administered in an amount below their individual ED₅₀.

13. A method according to claim 1, wherein said antagonist and said PPARα agonist are each administered in an amount below their individual ED₁₀.

14. A method according to claim 1, wherein at least one of said antagonist and said PPARα agonist is administered in an amount below its ED₁₀.

15. A method according to claim 1, wherein at least one of said antagonist and said PPARα agonist is administered in an amount below its ED₅₀.

16. A pharmaceutical composition for reducing food consumption in a mammal, said composition comprising a PPARα agonist and a cannabinoid CB1 receptor.

17. The composition according to claim 16, wherein the PPARα agonist is oleoylethanolamide.

18. The composition according to claim 17, wherein the antagonist is a pharmaceutically acceptable salt or solvate of a compound of the formula: 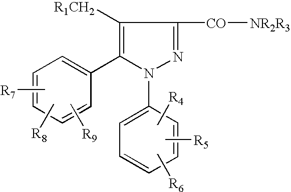
wherein R₁ is hydrogen, a fluorine, a hydroxyl, a (C₁-C₅)alkoxy, a (C₁-C₅)alkylthio, a hydroxy(C₁-C₅)alkoxy, a group —NR₁₀R₁₁, a cyano, a (C₁-C₅)alkylsulfonyl or a (C₁-C₅)alkylsulfinyl;
R₂ and R₃ are a (C₁-C₄)alkyl or, together with the nitrogen atom to which they are bonded, form a saturated or unsaturated 5- to 10-membered heterocyclic radical which is unsubstituted or monosubstituted or polysubstituted by a (C₁-C₃)alkyl or by a (C₁-C₃)alkoxy;
R₄, R₅, R₆, R₇, R₈ and R₉ are each independently hydrogen, a halogen or a trifluoromethyl, and if R₁ is a fluorine, R₄, R₅, R₆, R₇, R₈ and/or R₉ can also be a fluoromethyl, with the proviso that at least one of the substituents R₄ or R₇ is other than hydrogen; and
R₁₀ and R₁₁ are each independently hydrogen or a (C₁-C₅)alkyl, or R₁₀ and R₁₁, together with the nitrogen atom to which they are bonded, form a heterocyclic radical selected from pyrrolidin-1-yl, piperidin-1-yl, morpholin-4-yl and piperazin-1-yl, which is unsubstituted or substituted by a (C₁-C₄)alkyl.

19. The composition according to claim 17, wherein said antagonist is of the formula: 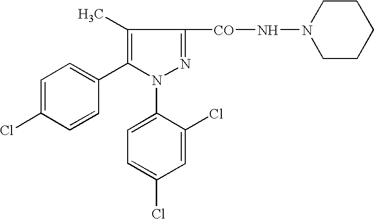
or a pharmaceutically acceptable salt thereof.

20. The composition according to claim 17, wherein the PPARα agonist is a fatty acid alkanolamide of the formula: 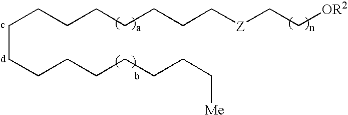
wherein n is any number from 0 to 5;
the sum of a and b can be any number from 0 to 4;
Z is a member selected from —C(O)N(R^{o})—; —(R^{o})NC(O)—; —OC(O)—; —(O)CO—; O; NR^{o}; and S, in which R^{o} and R² are independently selected from the group consisting of substituted or unsubstituted alkyl, hydrogen, substituted or unsubstituted C₁-C₆ alkyl, substituted or unsubstituted lower (C₁-C₆) acyl, homoalkyl, and aryl;
up to eight hydrogen atoms of the compound may also be substituted by methyl group or a double bond; and
the molecular bond between carbons c and d may be unsaturated or saturated.

21. The composition according to claim 17, wherein said composition is in a formulation suitable for administration by an oral, rectal, topical, or parenteral route of administration.

22. The composition according to claim 17, wherein said composition is in unit dosage format.

23. The composition according to claim 22, wherein at least one of said antagonist and said agonist is in an amount below its ED₁₀.

24. The composition according to claim 22, wherein at least one of said antagonist and said alkanolamide is in an amount below its ED₅₀.

25. The composition according to claim 16, wherein the antagonist has an IC₅₀ for the CB1 cannabinoid receptor which is less than one-fourth its IC₅₀ for the CB2 cannabinoid receptor.

26. The composition according to claim 20, wherein R⁰ and R² are members independently selected from the group comprising hydrogen, C₁-C₃ alkyl, and lower (C₁-C₃) acyl.

27. The composition according to claim 20, wherein a=1 and b=1.

28. The composition according to claim 20, wherein n=1.

29. The composition according to claim 20, wherein R¹ and R² are each H.

30. The composition according to claim 20, wherein the bond between carbon c and carbon d is a double bond.

31. The composition according to claim 20, wherein the alkanolamide or its homologue is according to one of the following formulae: 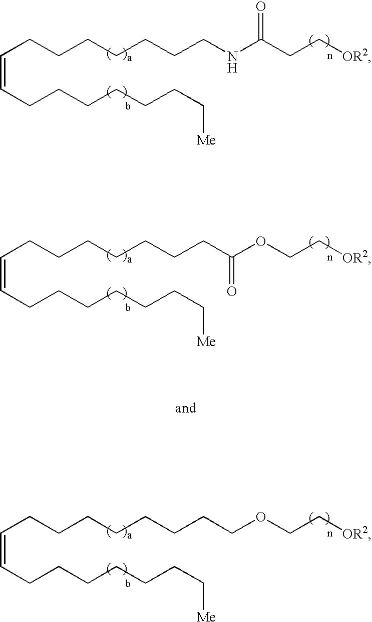
wherein n is from 1-5 and the sum of a and b is from 0 to 4; R² is selected from the group consisting of hydrogen, C₁-C₆ alkyl, and lower (C₁-C₆) acyl; and up to four hydrogen atoms of the fatty acid portion and alkanol portion thereof may also be substituted by methyl or a double bond.

32. A composition of claim 16, wherein the PPARα agonist is selected from the group consisting of clofibrate; fenofibrate, bezafibrate, gemfibrozil, and ciprofibrate.

33. A composition of claim 31, wherein the cannabinoid receptor antagonist is rimonabant.

34. A method of treating an appetency disorder in a human by administering a composition according to claim 17.

35. A method according to claim 34, wherein the appetite for a food, ethanol, or a psychoactive substance is to be reduced.
